SEQUENCE LISTING (312 sequences)

SEQ ID NO: 1 - DNA, 2029 bp, Phytophthora sojae
aagcttgaattctggcgttcatctccgacgagagccaacgctacggcattcgttccgtcatttcctcgcagcaactcgcttccacgatttctttgtattcactcggcagcctcgagctgattgagcgagagatggacgtccacgccgatggatcacccgtttggccgcaatatttgcctggactgttagatcccgtaccagagatagacatggtcagcgacggcttcattacctcgtcggaatcattgctggaacatcaaacaagttttatctacggcttcgataaccctgagtcgacggcatcatctggagcattggtagatgctgatgacgtgtgctcttccaccacttctggactgtgtatcaagctccggttcacaggcgccgatcagttggctccaatatttcttcgtaaactgcagagcattcacgctgcactatcttcgtcaattggtcttcctcccggatcaagtgttgctggttcaacttctacttatccggctccgtgggtgctggttggtgctcagagtctcggtggggatacttcgtggtccttcttcgtgggcgggtcgtcatcaagcggcaatgcccagagcttccgtctcacgttcgaagctgtaaactcgatgcaagatatgaccactaccacacccttcaccatagacctggagtttgttcctgacgacccaactgtcatccccattgtgcgacgtcggcagtttggcgacctcgcggtggtagacaccactggccagcctgaaccagcatttatcacctctgagattcctgatctgatcgacgatggagacgaagtcactggtatcaccataacattggatttggagcagcctcgacttggtggactgtattcaggttcacaactggatgatgtagccccaacttgcagcgagtgcacgacgttattggaggattgcagcagctcgcccgagtgccgagcgttctcagcgtgtatgggtgttctgcttgaggcagacttgacactgatagctacgatgctggagaaggacgatatcgacagcagcgttgatgcaacttggctgctgcaggactgcttgagtccatcggacggcacggaatggtcgagtacagtgcgtgagctgctcgagtccagctacgcttgtctgtgggcgaagagctgtccactggcatacagcaccacaatagggaggcagatcgtgctggactacagtcctggagagcagatcttgaccttcgacctggcgagcggattcgccagcgtcagcttcgagctcggcaactgggcgtacagttttgtcgaggacttcacggcgaacgcgacggaagtgacagcccaactggacacgatgctgatggagatgtaccgcactgcgctgtcgaatgtggcgacggtgaagtcggccctcgtcacgacggaagactcgaacacgggagtggtgacggcgcagttgaccattctctacgagttcctgggcgcactgccacagccttatgtcgcagagaatggaggttcgttggcgtgcaataccaccgaggaggctctgcatctgcgcgtactagccataacgtaagaacgagtgcaataacaagaacaacttcacttgcagtgcaagttgaagacactatagtggggaaagtgtcactcaagtggggtcacattttggttggccagtagtccgcgcccgctgtccagccaatcaaagtgtttgaagattccatctcctcttttttgcaccatacaacgcccttcatgcgggacttgttgcctacaataaccaagtctctccacgctggcaagctcgcagccaactatcctctcgcatgttgatcccaaggctgcgctcgccgacagcaacaagcctcactttctgctgactgctctgcttggaacatcgttcggggtaagtggcgacccccttgtctctctctgagcctcgcagctattggccccacatggctacatcgacagtctttacttactatagttttaattcgttcggtgacgctagcatctgagcatccacccggg

SEQ ID NO: 2 - protein, 138 aa, Phytophthora sojae
MRLSFVLSLVVAIGYVVTCNATEYSDETNIAMVESPDLVRRSLRNGDIAGGRFLRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 3 - protein, 138 aa, Phytophthora sojae
MRLSFVLSLVVAIGYVVTCNATEYSDETNIAMVESPDLVRAAAANGDIAGGRFLRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 4 - protein, 138 aa, Phytophthora sojae
MRLSFVLSLVVAIGYVVTCNATEYSDETNIAMVESPDLVRRSLRNGDIAGGAAAAAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 5 - protein, 138 aa, Phytophthora sojae
MRLSFVLSLVVAIGYVVTCNATEYSDETNIAMVESPDLVRAAAANGDIAGGAAAAAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 6 - protein, 138 aa, Phytophthora sojae
MRLSFVLSLVVAIGYVVTCNATEYSDETNIAMVESPDLVRRSLRNGDIAGGRFLRAHAAAAAGAATFSVTDLWNKVAAKKLAKAMLADPSKEQKAYEKWAKKGYSLDKIKNWLAIADPKQKGKYDRIYNGYTFHRYQS

SEQ ID NO: 7 - protein, 186 aa, Phytophthora sojae
MRLHYVGPVVAAALAASAHGLQVVSDSASDLQRTDEARQQPYINDKTKRFLTSEDKDLPLLVTSDGYASLLPQGGDNDRVLRSADIGDGDYEEERSKIKKRHKRKSHGHGVLDTFSVTDLWNKVAAKKLAKAMLADPSKEQKAYEKWAKKGYSLDKIKNWLAIADPKQKGKYDRIYNGYTFHRYQS

SEQ ID NO: 8 - protein, 195 aa, Phytophthora sojae
MGLHKGFFVAVALLALLIVAAPADAITDESQPRDATIVDAPLTGRGANARYLRTSTSIIKAPDAQLPSTKAAIASSVTKEEEERKISTGLSKLRQKLSKRFHDIPDWLLQLQAFLSVGLHHLTFSVTDLWNKVAAKKLAKAMLADPSKEQKAYEKWAKKGYSLDKIKNWLAIADPKQKGKYDRIYNGYTFHRYQS

SEQ ID NO: 9 - protein, 138 aa, Phytophthora sojae; identical to SEQ ID NO: 2

SEQ ID NO: 10 - protein, 138 aa, Phytophthora sojae; identical to SEQ ID NO: 3

SEQ ID NO: 11 - protein, 138 aa, Phytophthora sojae; identical to SEQ ID NO: 4

SEQ ID NO: 12 - protein, 138 aa, Phytophthora sojae; identical to SEQ ID NO: 5

SEQ ID NO: 13 - protein, 138 aa, Phytophthora sojae; identical to SEQ ID NO: 6

SEQ ID NO: 14 - protein, 138 aa, Phytophthora sojae; identical to SEQ ID NO: 2

SEQ ID NO: 15 - protein, 138 aa, Phytophthora sojae; identical to SEQ ID NO: 4

SEQ ID NO: 16 - protein, 645 aa, Phytophthora sojae
MATTKHLALAILVLLSIGMTTSARTLLDLRNRRTSLYPINTETRGVFDLNGVWNFKLDYGKGLEEKWYESKLTDTISMAVPSSYNDIGVTKEIRNHIGYVWYEREFTVPAYLKDQRIVLRFGSATHKAIVYVNGELVVEHKGGFLPFEAEINNSLRDGMNRVTVAVDNILDDSTLPVGLYSERHEEGLGKVIRNKPNFDFFNYAGLHRPVKIYTTPFTYVEDISVVTDFNGPTGTVTYTVDFQGKAETVKVSVVDEEGKVVASTEGLSGNVEIPNVILWEPLNTYLYQIKVELVNDGLTIDVYEEPFGVRTVEVNDGKFLINNKPFYFKGFGKHEDTPINGRGFNEASNVMDFNILKWIGANSFRTAHYPYSEELMRLADREGLVVIDETPAVGVHLNFMATTGLGEGSERVSTWEKIRTFEHHQDVLRELVSRDKNHPSVVMWSIANEAATEEEGAYEYFKPLVELTKELDPQKRPVTIVLFVMATPETDKVAELIDVIALNRYNGWYFDGGDLEAAKVHLRQEFHAWNKRCPGKPIMITEYGADTVAGFHDIDPVMFTEEYQVEYYQANHVVFDEFENFVGEQAWNFADFATSQGVMRVQGNKKGVFTRDRKPKLAAHVFRERWTNIPDFGYKNASHHHHHHV

SEQ ID NO: 17 - protein, 138 aa, Phytophthora sojae; identical to SEQ ID NO: 2

SEQ ID NO: 18 - protein, 138 aa, Phytophthora sojae; identical to SEQ ID NO: 4

SEQ ID NO: 19 - protein, 117 aa, Phytophthora sojae
TEYSDETNIAMVESPDLVRRSLRNGDIAGGRFLRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 20 - protein, 117 aa, Phytophthora sojae
TEYSDETNIAMVESPDLVRRSLRNGDIAGGAAAAAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 21 - protein, 138 aa, Phytophthora sojae; identical to SEQ ID NO: 2

SEQ ID NO: 22 - protein, 138 aa, Phytophthora sojae; identical to SEQ ID NO: 2

SEQ ID NO: 23 - protein, 117 aa, Phytophthora sojae
TEYSDETNIAMVESPDLVRRSLRNGDIAGGRFLRAHAAAAAGAATFSVTDLWNKVAAKKLAKAMLADPSKEQKAYEKWAKKGYSLDKIKNWLAIADPKQKGKYDRIYNGYTFHRYQS

SEQ ID NO: 24 - protein, 360 aa, Phytophthora sojae
TEYSDETNIAMVESPDLVRRSLRNGDIAGGRFLRAHEEDDAGERTFSVTDLWNKVAAKKLAKAMLADPSKEQKAYEKWAKKGYSLDKIKNWLAIADPKQKGKYDRIYNGYTFHRYQSGTATMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 25 - protein, 381 aa, Phytophthora sojae
MRLSFVLSLVVAIGYVVTCNATEYSDETNIAMVESPDLVRRSLRNGDIAGGRFLRAHEEDDAGERTFSVTDLWNKVAAKKLAKAMLADPSKEQKAYEKWAKKGYSLDKIKNWLAIADPKQKGKYDRIYNGYTFHRYQSGTATMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 26 - protein, 239 aa, Phytophthora sojae
MVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 27 - protein, 270 aa, Phytophthora sojae
MRLSFVLSLVVAIGYVVTCNATEYSDETNIAMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 28 - protein, 381 aa, Phytophthora sojae
MRLSFVLSLVVAIGYVVTCNATEYSDETNIAMVESPDLVRRSLRNGDIAGGAAAAAHEEDDAGERTFSVTDLWNKVAAKKLAKAMLADPSKEQKAYEKWAKKGYSLDKIKNWLAIADPKQKGKYDRIYNGYTFHRYQSGTATMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 29 - protein, 381 aa, Phytophthora sojae
MRLSFVLSLVVAIGYVVTCNATEYSDETNIAMVESPDLVRRSLRNGDIAGGRFLRAHAAAAAGAATFSVTDLWNKVAAKKLAKAMLADPSKEQKAYEKWAKKGYSLDKIKNWLAIADPKQKGKYDRIYNGYTFHRYQSGTATMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 30 - protein, 386 aa, Artificial Sequence
  Synthetic Sequence of 9 arginines  replacing the RXLR region of secreted Avr1b fused to GFP
MRLSFVLSLVVAIGYVVTCNATEYSDETNIAMVESPDLVRRSLRNGDIAGGRRRRRRRRRAHEEDDAGERTFSVTDLWNKVAAKKLAKAMLADPSKEQKAYEKWAKKGYSLDKIKNWLAIADPKQKGKYDRIYNGYTFHRYQSGTATMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 31 - protein, 388 aa, Artificial Sequence
  Synthetic sequence consisting of P. sojae Avr1b with a known TAT motif from Lentivirus HIV inserted in the position of the second RXLR sequence fused to GFP
MRLSFVLSLVVAIGYVVTCNATEYSDETNIAMVESPDLVRRSLRNGDIAGGYGRKKRRQRRRAHEEDDAGERTFSVTDLWNKVAAKKLAKAMLADPSKEQKAYEKWAKKGYSLDKIKNWLAIADPKQKGKYDRIYNGYTFHRYQSGTATMVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 32 - protein, 138 aa, Phytophthora sojae; identical to SEQ ID NO: 2

SEQ ID NO: 33 - protein, 138 aa, Phytophthora sojae
MRLSFVLSLVVAIGYVVTCNATEYSDETNIAMVESPDLVRRSLRNGDIAGGRFRLAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 34 - protein, 138 aa, Phytophthora sojae
MRLSFVLSLVVAIGYVVTCNATEYSDETNIAMVESPDLVRRSLRNGDIAGGFRLRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 35 - protein, 138 aa, Phytophthora sojae
MRLSFVLSLVVAIGYVVTCNATEYSDETNIAMVESPDLVRRSLRNGDIAGGRFARAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 36 - protein, 138 aa, Phytophthora sojae
MRLSFVLSLVVAIGYVVTCNATEYSDETNIAMVESPDLVRRSLRNGDIAGGQFLQAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 37 - protein, 138 aa, Phytophthora sojae
MRLSFVLSLVVAIGYVVTCNATEYSDETNIAMVESPDLVRRSLRNGDIAGGQFLRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 38 - protein, 138 aa, Phytophthora sojae
MRLSFVLSLVVAIGYVVTCNATEYSDETNIAMVESPDLVRRSLRNGDIAGGKFLRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 39 - protein, 138 aa, Phytophthora sojae
MRLSFVLSLVVAIGYVVTCNATEYSDETNIAMVESPDLVRRSLRNGDIAGGRFLQAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 40 - protein, 138 aa, Phytophthora sojae
MRLSFVLSLVVAIGYVVTCNATEYSDETNIAMVESPDLVRRSLRNGDIAGGRFVRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 41 - protein, 143 aa, Artificial Sequence
  Synthetic sequence consisting of P. sojae Avr1b with 9 arginines inserted where the second RXLR sequence is found.
MRLSFVLSLVVAIGYVVTCNATEYSDETNIAMVESPDLVRRSLRNGDIAGGRRRRRRRRRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 42 - protein, 146 aa, Artificial Sequence
  Synthetic sequence consisting of P. sojae Avr1b with a known TAT motif from Lentivirus HIV inserted in the position of the second RXLR sequence.
MRLSFVLSLVVAIGYVVTCNATEYSDETNIAMVESPDLVRRSLRNGDIAGGYGRKKKRRQRRRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 43 - protein, 138 aa, Artificial Sequence; identical to SEQ ID NO: 2
  Synthetic sequence consisting of P. sojae Avr1b with a RXLR motif from Plasmodium falciparum GBP protein inserted where the second RXLR sequence is found.

SEQ ID NO: 44 - protein, 132 aa, Artificial Sequence
  Synthetic sequence consisting of P. sojae Avr1b with a RXLRsequence of Plasmodium falciparum HRPII protein inserted where the second RXLR sequence is found.
MRLSFVLSLVVAIGYVVTCNAFNNNLCSKNAKGLNLNKRLLHETQAHVDDAHHAHHVADTFSVTDLWNKVAAKKLAKAMLADPSKEQKAYEKWAKKGYSLDKIKNWLAIADPKQKGKYDRIYNGYTFHRYQS

SEQ ID NO: 45 - protein, 135 aa, Artificial Sequence
  Synthetic sequence consisting of P. sojae Avr1b with a RXLRsequence from Plasmodium falciparum 1615c protein inserted where the second RXLR sequence is found.
MRLSFVLSLVVAIGYVVTCNASYNKINSSSTYTHSRILKQLEFITLEEKTVNALQEMLDDSVTFSVTDLWNKVAAKKLAKAMLADPSKEQKAYEKWAKKGYSLDKIKNWLAIADPKQKGKYDRIYNGYTFHRYQS

SEQ ID NO: 46 - protein, 365 aa, Phytophthora sojae
MRGSHHHHHHGMASMTGGQQMGRDLYDDDDKDRWGSRSTEYSDETNIAMVESPDLVRRSLRNGDIAGGRFLRAHEEDDAGERTFSVTDEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 47 - protein, 365 aa, Phytophthora sojae
MRGSHHHHHHGMASMTGGQQMGRDLYDDDDKDRWGSRSTEYSDETNIAMVESPDLVRAAAANGDIAGGAAAAAHEEDDAGERTFSVTDEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 48 - protein, 365 aa, Phytophthora sojae
MRGSHHHHHHGMASMTGGQQMGRDLYDDDDKDRWGSRSTEYSDETNIAMVESPDLVRRSLRNGDIAGGRFLRAHAAAAAGAATFSVTDEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 49 - DNA, 31 bp, Artificial Sequence
  Synthetically constructed DNA sequence to amplify genes from genomes or plasmids with various restriction sites to faciliate cloning
ataagcttgaatctggcgttcatctccgacg

SEQ ID NO: 50 - DNA, 30 bp, Artificial Sequence
  Synthetically constructed DNA sequence to amplify genes from genomes or plasmids with various restriction sites to faciliate cloning
ttcccgggtggatgctcagatgctagcgtc

SEQ ID NO: 51 - DNA, 20 bp, Artificial Sequence
  Synthetically constructed DNA sequence to amplify genes from genomes or plasmids with various restriction sites to faciliate cloning
ttctccttttcactctcacg

SEQ ID NO: 52 - DNA, 21 bp, Artificial Sequence
  Synthetically constructed DNA sequence to amplify genes from genomes or plasmids with various restriction sites to faciliate cloning
agacacaaaatctgcaacttc

SEQ ID NO: 53 - DNA, 20 bp, Artificial Sequence
  Synthetically constructed DNA sequence to amplify genes from genomes or plasmids with various restriction sites to faciliate cloning
accttcagcgtgactgacct

SEQ ID NO: 54 - DNA, 19 bp, Artificial Sequence
  Synthetically constructed DNA sequence to amplify genes from genomes or plasmids with various restriction sites to faciliate cloning
gcgattgccaaccagttct

SEQ ID NO: 55 - DNA, 20 bp, Artificial Sequence
  Synthetically constructed DNA sequence to amplify genes from genomes or plasmids with various restriction sites to faciliate cloning
cgacatccgtaaggacctgt

SEQ ID NO: 56 - DNA, 20 bp, Artificial Sequence
  Synthetically constructed DNA sequence to amplify genes from genomes or plasmids with various restriction sites to faciliate cloning
ttcgagatccacatctgctg

SEQ ID NO: 57 - DNA, 35 bp, Artificial Sequence
  Synthetically constructed DNA sequence to amplify genes from genomes or plasmids with various restriction sites to faciliate cloning
ggggtaccgacaacaatgcgtctatcttttgtgct

SEQ ID NO: 58 - DNA, 28 bp, Artificial Sequence
  Synthetically constructed DNA sequence to amplify genes from genomes or plasmids with various restriction sites to faciliate cloning
ggggtacctcagctctgataccggtgaa

SEQ ID NO: 59 - DNA, 62 bp, Artificial Sequence
  Synthetically constructed DNA sequence to amplify genes from genomes or plasmids with various restriction sites to faciliate cloning
atcgcactcgagctttcgcagatcccggggggcaatgagatatgactgagtactccgacgaa

SEQ ID NO: 60 - DNA, 63 bp, Artificial Sequence
  Synthetically constructed DNA sequence to amplify genes from genomes or plasmids with various restriction sites to faciliate cloning
atcgcactcgagcttgtcgatcgacagatccggtcggcatctacttcagctctgataccggtg

SEQ ID NO: 61 - DNA, 59 bp, Artificial Sequence
  Synthetically constructed DNA sequence to amplify genes from genomes or plasmids with various restriction sites to faciliate cloning
atcgcactcgagctttcgcagatcccggggggcaatgagatatgcgtctatcttttgtg

SEQ ID NO: 62 - DNA, 40 bp, Artificial Sequence
  Synthetically constructed DNA sequence to amplify genes from genomes or plasmids with various restriction sites to faciliate cloning
tcgtccgtgctgcagctgctaacggcgacattgccggtgg

SEQ ID NO: 63 - DNA, 40 bp, Artificial Sequence
  Synthetically constructed DNA sequence to amplify genes from genomes or plasmids with various restriction sites to faciliate cloning
tcgccgttagcagctgcagcacggacgagatctggagatt

SEQ ID NO: 64 - DNA, 40 bp, Artificial Sequence
  Synthetically constructed DNA sequence to amplify genes from genomes or plasmids with various restriction sites to faciliate cloning
ccggtggagctgcagctgctgctcatgaagaggacgatgc

SEQ ID NO: 65 - DNA, 40 bp, Artificial Sequence
  Synthetically constructed DNA sequence to amplify genes from genomes or plasmids with various restriction sites to faciliate cloning
tcatgagcagcagctgcagctccaccggcaatgtcgccgt

SEQ ID NO: 66 - DNA, 65 bp, Artificial Sequence
  Synthetically constructed DNA sequence to amplify genes from genomes or plasmids with various restriction sites to faciliate cloning
gctgcagctgctaacggcgacattgccggtggagctgcagctgctgctcatgaagaggacgatgc

SEQ ID NO: 67 - DNA, 65 bp, Artificial Sequence
  Synthetically constructed DNA sequence to amplify genes from genomes or plasmids with various restriction sites to faciliate cloning
agcagctgcagctccaccggcaatgtcgccgttagcagctgcagcacggacgagatctggagatt

SEQ ID NO: 68 - DNA, 44 bp, Artificial Sequence
  Synthetically constructed DNA sequence to amplify genes from genomes or plasmids with various restriction sites to faciliate cloning
gctgcagcagctgcgggggctgctaccttcagcgtgactgacct

SEQ ID NO: 69 - DNA, 44 bp, Artificial Sequence
  Synthetically constructed DNA sequence to amplify genes from genomes or plasmids with various restriction sites to faciliate cloning
agcagcccccgcagctgctgcagcatgagctcgaagaaatcttc

SEQ ID NO: 70 - DNA, 34 bp, Artificial Sequence
  Synthetically constructed DNA sequence to amplify genes from genomes or plasmids with various restriction sites to faciliate cloning
attcccggggacaacaatgcgactccactacgtg

SEQ ID NO: 71 - DNA, 38 bp, Artificial Sequence
  Synthetically constructed DNA sequence to amplify genes from genomes or plasmids with various restriction sites to faciliate cloning
gtcagtcacgctgaaggtatcgagaacgccatgcccat

SEQ ID NO: 72 - DNA, 35 bp, Artificial Sequence
  Synthetically constructed DNA sequence to amplify genes from genomes or plasmids with various restriction sites to faciliate cloning
attcccggggacaacaatgggcctccacaagggct

SEQ ID NO: 73 - DNA, 36 bp, Artificial Sequence
  Synthetically constructed DNA sequence to amplify genes from genomes or plasmids with various restriction sites to faciliate cloning
gtcagtcacgctgaaggttaggtggtgtagtccgac

SEQ ID NO: 74 - DNA, 35 bp, Artificial Sequence
  Synthetically constructed DNA sequence to amplify genes from genomes or plasmids with various restriction sites to faciliate cloning
aacccgggacaacaatgaccttcagcgtgactgac

SEQ ID NO: 75 - DNA, 28 bp, Artificial Sequence
  Synthetically constructed DNA sequence to amplify genes from genomes or plasmids with various restriction sites to faciliate cloning
ggaggaattcgctggctggtggcaggat

SEQ ID NO: 76 - DNA, 23 bp, Artificial Sequence
  Synthetically constructed DNA sequence to amplify genes from genomes or plasmids with various restriction sites to faciliate cloning
gtattggctagagaagcttgcca

SEQ ID NO: 77 - DNA, 57 bp, Artificial Sequence
  Synthetically constructed DNA sequence to amplify genes from genomes or plasmids with various restriction sites to faciliate cloning
agaaactcgagcttgtcgatcgacagatccggtcggcaggtacctcagctctgatac

SEQ ID NO: 78 - DNA, 24 bp, Artificial Sequence
  Synthetically constructed DNA sequence to amplify genes from genomes or plasmids with various restriction sites to faciliate cloning
gaagatttcgacttgctcatgaag

SEQ ID NO: 79 - DNA, 24 bp, Artificial Sequence
  Synthetically constructed DNA sequence to amplify genes from genomes or plasmids with various restriction sites to faciliate cloning
cttcatgagcaagtcgaaatcttc

SEQ ID NO: 80 - DNA, 26 bp, Artificial Sequence
  Synthetically constructed DNA sequence to amplify genes from genomes or plasmids with various restriction sites to faciliate cloning
tgccggtggatttagacttcgagctc

SEQ ID NO: 81 - DNA, 26 bp, Artificial Sequence
  Synthetically constructed DNA sequence to amplify genes from genomes or plasmids with various restriction sites to faciliate cloning
gagctcgaagtctaaatccaccggca

SEQ ID NO: 82 - DNA, 20 bp, Artificial Sequence
  Synthetically constructed DNA sequence to amplify genes from genomes or plasmids with various restriction sites to faciliate cloning
gaagatttgcacgagctcat

SEQ ID NO: 83 - DNA, 20 bp, Artificial Sequence
  Synthetically constructed DNA sequence to amplify genes from genomes or plasmids with various restriction sites to faciliate cloning
atgagctcgtgcaaatcttc

SEQ ID NO: 84 - DNA, 28 bp, Artificial Sequence
  Synthetically constructed DNA sequence to amplify genes from genomes or plasmids with various restriction sites to faciliate cloning
ggtggacagtttcttcaagctcatgaag

SEQ ID NO: 85 - DNA, 28 bp, Artificial Sequence
  Synthetically constructed DNA sequence to amplify genes from genomes or plasmids with various restriction sites to faciliate cloning
agcttgaagaaactgtccaccggcaatg

SEQ ID NO: 86 - DNA, 52 bp, Artificial Sequence
  Synthetically constructed DNA sequence to amplify genes from genomes or plasmids with various restriction sites to faciliate cloning
ctttcaccggtatcagagcggtaccgccaccatggtgagcaagggcgccgag

SEQ ID NO: 87 - DNA, 29 bp, Artificial Sequence
  Synthetically constructed DNA sequence to amplify genes from genomes or plasmids with various restriction sites to faciliate cloning
aaggtacctcacttgtacagctcatccat

SEQ ID NO: 88 - DNA, 33 bp, Artificial Sequence
  Synthetically constructed DNA sequence to amplify genes from genomes or plasmids with various restriction sites to faciliate cloning
tcatgagctcgaagaaatcttccgccggcaatg

SEQ ID NO: 89 - DNA, 27 bp, Artificial Sequence
  Synthetically constructed DNA sequence to amplify genes from genomes or plasmids with various restriction sites to faciliate cloning
cattgccggcggaagatttcttcaagc

SEQ ID NO: 90 - DNA, 20 bp, Artificial Sequence
  Synthetically constructed DNA sequence to amplify genes from genomes or plasmids with various restriction sites to faciliate cloning
cattgccggcggacaatttc

SEQ ID NO: 91 - DNA, 23 bp, Artificial Sequence
  Synthetically constructed DNA sequence to amplify genes from genomes or plasmids with various restriction sites to faciliate cloning
cattgccggcggaagatttgttc

SEQ ID NO: 92 - DNA, 23 bp, Artificial Sequence
  Synthetically constructed DNA sequence to amplify genes from genomes or plasmids with various restriction sites to faciliate cloning
cattgccggcggaaagtttcttc

SEQ ID NO: 93 - DNA, 37 bp, Artificial Sequence
  Synthetically constructed DNA sequence to amplify genes from genomes or plasmids with various restriction sites to faciliate cloning
cgtcgacgtcggcgacgcgctcatgaagaggacgatg

SEQ ID NO: 94 - DNA, 39 bp, Artificial Sequence
  Synthetically constructed DNA sequence to amplify genes from genomes or plasmids with various restriction sites to faciliate cloning
cattgccggcggacgacggcgacgtcgacgtcggcgacg

SEQ ID NO: 95 - DNA, 44 bp, Artificial Sequence
  Synthetically constructed DNA sequence to amplify genes from genomes or plasmids with various restriction sites to faciliate cloning
taagaaacgccgtcagcgacgtcgagctcatgaagaggacgatg

SEQ ID NO: 96 - DNA, 38 bp, Artificial Sequence
  Synthetically constructed DNA sequence to amplify genes from genomes or plasmids with various restriction sites to faciliate cloning
cattgccggcggatatggacgtaagaaacgccgtcagc

SEQ ID NO: 97 - DNA, 56 bp, Artificial Sequence
  Synthetically constructed DNA sequence to amplify genes from genomes or plasmids with various restriction sites to faciliate cloning
gaaggagaagactacactccggaaaagcaagcaaaagaccttcagcgtgactgacc

SEQ ID NO: 98 - DNA, 55 bp, Artificial Sequence
  Synthetically constructed DNA sequence to amplify genes from genomes or plasmids with various restriction sites to faciliate cloning
ctcgtatactggcagagggcgaagatacctgcgcaaggaaggagaagactacact

SEQ ID NO: 99 - DNA, 52 bp, Artificial Sequence
  Synthetically constructed DNA sequence to amplify genes from genomes or plasmids with various restriction sites to faciliate cloning
gtatgagaaagcggtagattacggctttcgagagtctcgtatactggcagag

SEQ ID NO: 100 - DNA, 40 bp, Artificial Sequence
  Synthetically constructed DNA sequence to amplify genes from genomes or plasmids with various restriction sites to faciliate cloning
ctaccgctttctcatacttatctgcgttgcaggtcacgac

SEQ ID NO: 101 - DNA, 56 bp, Artificial Sequence
  Synthetically constructed DNA sequence to amplify genes from genomes or plasmids with various restriction sites to faciliate cloning
tgtcgacgatgcgcaccatgcacaccatgttgcagataccttcagcgtgactgacc

SEQ ID NO: 102 - DNA, 55 bp, Artificial Sequence
  Synthetically constructed DNA sequence to amplify genes from genomes or plasmids with various restriction sites to faciliate cloning
aacctcaacaagagactgttgcacgagacacaagcacatgtcgacgatgcgcacc

SEQ ID NO: 103 - DNA, 53 bp, Artificial Sequence
  Synthetically constructed DNA sequence to amplify genes from genomes or plasmids with various restriction sites to faciliate cloning
acaataacctgtgtagtaagaatgctaaaggcttgaacctcaacaagagactg

SEQ ID NO: 104 - DNA, 39 bp, Artificial Sequence
  Synthetically constructed DNA sequence to amplify genes from genomes or plasmids with various restriction sites to faciliate cloning
actacacaggttattgttaaatgcgttgcaggtcacgac

SEQ ID NO: 105 - DNA, 57 bp, Artificial Sequence
  Synthetically constructed DNA sequence to amplify genes from genomes or plasmids with various restriction sites to faciliate cloning
cagtcaatgcattacaagaaatgttagatgatagtgtcaccttcagcgtgactgacc

SEQ ID NO: 106 - DNA, 55 bp, Artificial Sequence
  Synthetically constructed DNA sequence to amplify genes from genomes or plasmids with various restriction sites to faciliate cloning
ctcaagcagttggagttcatcacattggaagagaagacagtcaatgcattacaag

SEQ ID NO: 107 - DNA, 57 bp, Artificial Sequence
  Synthetically constructed DNA sequence to amplify genes from genomes or plasmids with various restriction sites to faciliate cloning
aagatcaactcgtcatctacttatacacacagtagaatactcaagcagttggagttc

SEQ ID NO: 108 - DNA, 45 bp, Artificial Sequence
  Synthetically constructed DNA sequence to amplify genes from genomes or plasmids with various restriction sites to faciliate cloning
agatgacgagttgatcttgttgtaacttgcgttgcaggtcacgac

SEQ ID NO: 109 - DNA, 29 bp, Artificial Sequence
  Synthetically constructed DNA sequence to amplify genes from genomes or plasmids with various restriction sites to faciliate cloning
gatctagatctgtggaatctccagatctc

SEQ ID NO: 110 - DNA, 39 bp, Artificial Sequence
  Synthetically constructed DNA sequence to amplify genes from genomes or plasmids with various restriction sites to faciliate cloning
gtcatatggatagccggacatgtcagtcacgctgaaggt

SEQ ID NO: 111 - DNA, 47 bp, Artificial Sequence
  Synthetically constructed DNA sequence to amplify genes from genomes or plasmids with various restriction sites to faciliate cloning
gatcccatggagccagcatagtctgggacgtcatatggatagccgga

SEQ ID NO: 112 - protein, 324 aa, Artificial Sequence
  Synthetic construct fusing 9 arginine residues to a linker sequence to humanized GFP
MRGSHHHHHHGMASMTGGQQMGRDLYDDDDKDRWGSRSRRRRRRRRREFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 113 - protein, 359 aa, Phytophthora sojae
MRGSHHHHHHGMASMTGGQQMGRDLYDDDDKDRWGSRSTRVPDDANLQSVNAPVQTVTRSRRFLRTADTDIVYEPKVHNPGKEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 114 - protein, 359 aa, Phytophthora sojae
MRGSHHHHHHGMASMTGGQQMGRDLYDDDDKDRWGSRSTRVPDDANLQSVNAPVQTVTRSRAAAATADTDIVYEPKVHNPGKEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 115 - protein, 359 aa, Phytophthora sojae
MRGSHHHHHHGMASMTGGQQMGRDLYDDDDKDRWGSRSTRVPDDANLQSVNAPVQTVTRSRRFLRTAATAIVYAPAVHNPGKEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 116 - protein, 403 aa, Phytophthora sojae
MRGSHHHHHHGMASMTGGQQMGRDLYDDDDKDRWGSRSLTCATSEQQTRPELCFFFSVRSSWPSTISDGACLALVSAEQGATAGRNTLSLRSMMATEDMATSTRSLRSQATNVDDDANVSIENRGMEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 117 - protein, 403 aa, Phytophthora sojae
MRGSHHHHHHGMASMTGGQQMGRDLYDDDDKDRWGSRSLTCATSEQQTRPELCFFFSVRSSWPSTISDGACLALVSAEQGATAGRNTLSLRSMMATEDMATSTAAAASQATNVDDDANVSIENRGMEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 118 - protein, 403 aa, Phytophthora sojae
MRGSHHHHHHGMASMTGGQQMGRDLYDDDDKDRWGSRSLTCATSEQQTRPELCFFFSVRSSWPSTISDGACLALVSAEQGATAGRNTLSLRSMMATEDMATSTRSLRSQATNVAAAANVSIANAGMEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 119 - protein, 359 aa, Melampsora lini
MRGSHHHHHHGMASMTGGQQMGRDLYDDDDKDRWGSRSMEHVPAELTRVSEGYTRFYRSPTASVILSGLVKVKWDNEQMTMPEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 120 - protein, 359 aa, Melampsora lini
MRGSHHHHHHGMASMTGGQQMGRDLYDDDDKDRWGSRSMEHVPAELTRVSEGYTAAAASPTASVILSGLVKVKWANAQMTMPEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 121 - protein, 353 aa, Plasmodium falciparum
MRGSHHHHHHGMASMTGGQQMGRDLYDDDDKDRWGSRSDKYEKAVDYGFRESRILAEGEDTCARKEKTTLRKSKQKEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 122 - protein, 353 aa, Phytophthora sojae
MRGSHHHHHHGMASMTGGQQMGRDLYDDDDKDRWGSRSDKYEKAVDYGFRESAAAAAGEDTCARKEKTTLRKSKQKEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 123 - protein, 353 aa, Plasmodium falciparum
MRGSHHHHHHGMASMTGGQQMGRDLYDDDDKDRWGSRSFNNNLCSKNAKGLNLNKRLLHETQAHVDDAHHAHHVADEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 124 - protein, 353 aa, Plasmodium falciparum
MRGSHHHHHHGMASMTGGQQMGRDLYDDDDKDRWGSRSFNNNLCSKNAKGLNLNKAAAAATQAHVDDAHHAHHVADEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 125 - protein, 356 aa, Plasmodium falciparum
MRGSHHHHHHGMASMTGGQQMGRDLYDDDDKDRWGSRSSYNKINSSSTYTHSRILKQLEFITLEEKTVNALQEMLDDSVEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 126 - protein, 356 aa, Plasmodium falciparum
MRGSHHHHHHGMASMTGGQQMGRDLYDDDDKDRWGSRSSYNKINSSSTYTHSAAAAALEFITLEEKTVNALQEMLDDSVEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 127 - protein, 485 aa, Phytophthora sojae
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSLTCATSEQQTRPELCFFFSVRSSWPSTISDGACLALVSAEQGATAGRNTLSLRSMMATEDMATSTRSLRSQATNVDDDANVSIENRGMNPSVLTKLGEFASTLTAGNTANKLWLMADVDPKSAFKLLGLDMPGVRFIDNPKMLQWLKFTKAYLDMKKSGFGETSAHALLYEKIGGPDLSLLLLSLKDAPDANSLVQKLTNSQFGMWHDARIEPEQLAQTVFKIQDVRKLPKNDPKLQVIDDYAKYHRKHRKFLNSIMII

SEQ ID NO: 128 - protein, 485 aa, Phytophthora sojae
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSLTCATSEQQTRPELCFFFSVRSSWPSTISDGACLALVSAEQGATAGRNTLSLRSMMATEDMATSTAAAASQATNVDDDANVSIENRGMNPSVLTKLGEFASTLTAGNTANKLWLMADVDPKSAFKLLGLDMPGVRFIDNPKMLQWLKFTKAYLDMKKSGFGETSAHALLYEKIGGPDLSLLLLSLKDAPDANSLVQKLTNSQFGMWHDARIEPEQLAQTVFKIQDVRKLPKNDPKLQVIDDYAKYHRKHRKFLNSIMII

SEQ ID NO: 129 - protein, 342 aa, Phytophthora sojae
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSTRVPDDANLQSVNAPVQTVTRSRRFLRTADTDIVYEPKVHNPGKKQVFIEDKLQKALTDPKKNKKLYARWYNSGFTVKQVEGGLDQNENRELELTYKNLALGYAKYYQARRSQEAK

SEQ ID NO: 130 - protein, 342 aa, Phytophthora sojae
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSTRVPDDANLQSVNAPVQTVTRSRAAAATADTDIVYEPKVHNPGKKQVFIEDKLQKALTDPKKNKKLYARWYNSGFTVKQVEGGLDQNENRELELTYKNLALGYAKYYQARRSQEAK

SEQ ID NO: 131 - protein, 342 aa, Phytophthora sojae
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSTRVPDDANLQSVNAPVQTVTRSRRFLRTAATAIVYAPAVHNPGKKQVFIEDKLQKALTDPKKNKKLYARWYNSGFTVKQVEGGLDQNENRELELTYKNLALGYAKYYQARRSQEAK

SEQ ID NO: 132 - protein, 279 aa, Phytophthora sojae
MMQWSAILIRTCFSGSGGEALTCATSEQQTRPELCFFFSVRSSWPSTISDGACLALVSAEQGATAGRNTLSLRSMMATEDMATSTRSLRSQATNVDDDANVSIENRGMNPSVLTKLGEFASTLTAGNTANKLWLMADVDPKSAFKLLGLDMPGVRFIDNPKMLQWLKFTKAYLDMKKSGFGETSAHALLYEKIGGPDLSLLLLSLKDAPDANSLVQKLTNSQFGMWHDARIEPEQLAQTVFKIQDVRKLPKNDPKLQVIDDYAKYHRKHRKFLNSIMII

SEQ ID NO: 133 - protein, 279 aa, Phytophthora sojae
MMQWSAILIRTCFSGSGGEALTCATSEQQTRPELCFFFSVRSSWPSTISDGACLALVSAEQGATAGRNTLSLRSMMATEDMATSTAAAASQATNVDDDANVSIENRGMNPSVLTKLGEFASTLTAGNTANKLWLMADVDPKSAFKLLGLDMPGVRFIDNPKMLQWLKFTKAYLDMKKSGFGETSAHALLYEKIGGPDLSLLLLSLKDAPDANSLVQKLTNSQFGMWHDARIEPEQLAQTVFKIQDVRKLPKNDPKLQVIDDYAKYHRKHRKFLNSIMII

SEQ ID NO: 134 - protein, 279 aa, Phytophthora sojae
MMQWSAILIRTCFSGSGGEALTCATSEQQTRPELCFFFSVRSSWPSTISDGACLALVSAEQGATAGRNTLSLRSMMATEDMATSTRSLRSQATNVAAAANVSIANAGMNPSVLTKLGEFASTLTAGNTANKLWLMADVDPKSAFKLLGLDMPGVRFIDNPKMLQWLKFTKAYLDMKKSGFGETSAHALLYEKIGGPDLSLLLLSLKDAPDANSLVQKLTNSQFGMWHDARIEPEQLAQTVFKIQDVRKLPKNDPKLQVIDDYAKYHRKHRKFLNSIMII

SEQ ID NO: 135 - protein, 259 aa, Phytophthora sojae
LTCATSEQQTRPELCFFFSVRSSWPSTISDGACLALVSAEQGATAGRNTLSLRSMMATEDMATSTRSLRSQATNVDDDANVSIENRGMNPSVLTKLGEFASTLTAGNTANKLWLMADVDPKSAFKLLGLDMPGVRFIDNPKMLQWLKFTKAYLDMKKSGFGETSAHALLYEKIGGPDLSLLLLSLKDAPDANSLVQKLTNSQFGMWHDARIEPEQLAQTVFKIQDVRKLPKNDPKLQVIDDYAKYHRKHRKFLNSIMII

SEQ ID NO: 136 - protein, 259 aa, Phytophthora sojae
LTCATSEQQTRPELCFFFSVRSSWPSTISDGACLALVSAEQGATAGRNTLSLRSMMATEDMATSTAAAASQATNVDDDANVSIENRGMNPSVLTKLGEFASTLTAGNTANKLWLMADVDPKSAFKLLGLDMPGVRFIDNPKMLQWLKFTKAYLDMKKSGFGETSAHALLYEKIGGPDLSLLLLSLKDAPDANSLVQKLTNSQFGMWHDARIEPEQLAQTVFKIQDVRKLPKNDPKLQVIDDYAKYHRKHRKFLNSIMII

SEQ ID NO: 137 - protein, 259 aa, Phytophthora sojae
LTCATSEQQTRPELCFFFSVRSSWPSTISDGACLALVSAEQGATAGRNTLSLRSMMATEDMATSTRSLRSQATNVAAAANVSIANAGMNPSVLTKLGEFASTLTAGNTANKLWLMADVDPKSAFKLLGLDMPGVRFIDNPKMLQWLKFTKAYLDMKKSGFGETSAHALLYEKIGGPDLSLLLLSLKDAPDANSLVQKLTNSQFGMWHDARIEPEQLAQTVFKIQDVRKLPKNDPKLQVIDDYAKYHRKHRKFLNSIMII

SEQ ID NO: 138 - protein, 137 aa, Artificial Sequence
  Synthetic Sequence of amino acids 24-67 of avrL567 replacing theRXLR region of seceted Avr1b
MRLSFVLSLVVAIGYVVTCNAMEHVPAELTRVSEGYTRFYRSPTASVILSGLVKVKWDNEQMTMTFSVTDLWNKVAAKKLAKAMLADPSKEQKAYEKWAKKGYSLDKIKNWLAIADPKQKGKYDRIYNGYTFHRYQS

SEQ ID NO: 139 - protein, 137 aa, Artificial Sequence
  Synthetic Sequence of amino acids 24-67 of avrL567 replacing theRXLR region of secreted Avr1b. RFYR-DE mutated to AAAA-AA
MRLSFVLSLVVAIGYVVTCNAMEHVPAELTRVSEGYTAAAASPTASVILSGLVKVKWANAQMTMTFSVTDLWNKVAAKKLAKAMLADPSKEQKAYEKWAKKGYSLDKIKNWLAIADPKQKGKYDRIYNGYTFHRYQS

SEQ ID NO: 140 - protein, 116 aa, Artificial Sequence
  Synthetic Sequence of amino acids 24-67 of avrL567 replacing theRXLR regio n of mature Avr1b.
MEHVPAELTRVSEGYTRFYRSPTASVILSGLVKVKWDNEQMTMTFSVTDLWNKVAAKKLAKAMLADPSKEQKAYEKWAKKGYSLDKIKNWLAIADPKQKGKYDRIYNGYTFHRYQS

SEQ ID NO: 141 - protein, 116 aa, Artificial Sequence
  Synthetic Sequence of amino acids 24-67 of avrL567 replacing theRXLR region of mature Avr1b. RFYR-DE mutated to AAAA-AA
MEHVPAELTRVSEGYTAAAASPTASVILSGLVKVKWANAQMTMTFSVTDLWNKVAAKKLAKAMLADPSKEQKAYEKWAKKGYSLDKIKNWLAIADPKQKGKYDRIYNGYTFHRYQS

SEQ ID NO: 142 - protein, 169 aa, Artificial Sequence
  Synthetic Sequence of the N-terminus of AvrM replacing the RXLR region of secreted Avr1b.
MRLSFVLSLVVAIGYVVTCNAHPVYDYNPIPNHIHGDLKRRAYIERYSQCSDSQASEIRAALKSCAELASWGYHAVKNDNRLFKLIFKTDSTDIQNTFSVTDLWNKVAAKKLAKAMLADPSKEQKAYEKWAKKGYSLDKIKNWLAIADPKQKGKYDRIYNGYTFHRYQS

SEQ ID NO: 143 - protein, 192 aa, Artificial Sequence
  Synthetic Sequence of the N-terminus of AvrPita replacing theRXLR region of secreted Avr1b.
MRLSFVLSLVVAIGYVVTCNANNLGTVPDVPHQIPNDKSGTPAIEDPKDMKGFNKALKSTPESEKLGTSSVEGIPQPEFDRGFLRPFGAKMKFLKPDQVQKLSTDDLITYMAEKDKNVRTFSVTDLWNKVAAKKLAKAMLADPSKEQKAYEKWAKKGYSLDKIKNWLAIADPKQKGKYDRIYNGYTFHRYQS

SEQ ID NO: 144 - protein, 142 aa, Phytophthora sojae
MRLSFVLSLVVAIGYVVTCNATEYSDETNIAMVESPDLVRRSLRNGDIAGGKRRAYIERAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 145 - protein, 139 aa, Phytophthora sojae
MRLSFVLSLVVAIGYVVTCNATEYSDETNIAMVESPDLVRRSLRNGDIAGGRAALKAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 146 - protein, 149 aa, Phytophthora sojae
MRLSFVLSLVVAIGYVVTCNATEYSDETNIAMVESPDLVRRSLRNGDIAGGRLFKLIFKTDSTDIQAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 147 - protein, 138 aa, Phytophthora sojae
MRLSFVLSLVVAIGYVVTCNATEYSDETNIAMVESPDLVRRSLRNGDIAGGHFLRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 148 - protein, 138 aa, Phytophthora sojae
MRLSFVLSLVVAIGYVVTCNATEYSDETNIAMVESPDLVRRSLRNGDIAGGRFFRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 149 - protein, 138 aa, Phytophthora sojae
MRLSFVLSLVVAIGYVVTCNATEYSDETNIAMVESPDLVRRSLRNGDIAGGRFYRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 150 - protein, 138 aa, Phytophthora sojae
MRLSFVLSLVVAIGYVVTCNATEYSDETNIAMVESPDLVRRSLRNGDIAGGRFIRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 151 - protein, 138 aa, Phytophthora sojae
MRLSFVLSLVVAIGYVVTCNATEYSDETNIAMVESPDLVRRSLRNGDIAGGRFMRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 152 - protein, 138 aa, Phytophthora sojae
MRLSFVLSLVVAIGYVVTCNATEYSDETNIAMVESPDLVRRSLRNGDIAGGRFLGAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 153 - protein, 138 aa, Phytophthora sojae
MRLSFVLSLVVAIGYVVTCNATEYSDETNIAMVESPDLVRRSLRNGDIAGGRMVRAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 154 - protein, 138 aa, Phytophthora sojae
MRLSFVLSLVVAIGYVVTCNATEYSDETNIAMVESPDLVRRSLRNGDIAGGRLGTAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 155 - protein, 138 aa, Phytophthora sojae
MRLSFVLSLVVAIGYVVTCNATEYSDETNIAMVESPDLVRRSLRNGDIAGGRLTQAHEEDDAGERTFSVTDLWNKVAAKKLAKAMLADPSKEQKAYEKWAKKGYSLDKIKNWLAIADPKQKGKYDRIYNGYTFHRYQS

SEQ ID NO: 156 - DNA, 53 bp, Artificial Sequence
  Synthetically constructed DNA sequence to amplify genes from genomes or plasmids with various restriction sites to faciliate cloning
gcatcccatggagccagcatagtctgggacgtcatatggatagccggacatgg

SEQ ID NO: 157 - DNA, 32 bp, Artificial Sequence
  Synthetically constructed DNA sequence to amplify genes from genomes or plasmids with various restriction sites to faciliate cloning
atgcagatctctcacttgcgccacctccgagc

SEQ ID NO: 158 - DNA, 48 bp, Artificial Sequence
  Synthetically constructed DNA sequence to amplify genes from genomes or plasmids with various restriction sites to faciliate cloning
catatggatagccggacatggtggatctgtcgactctgttttcaatcg

SEQ ID NO: 159 - DNA, 46 bp, Artificial Sequence
  Synthetically constructed DNA sequence to amplify genes from genomes or plasmids with various restriction sites to faciliate cloning
catatggatagccggacatggtggatctgtcgactgcgtttgcaat

SEQ ID NO: 160 - DNA, 27 bp, Artificial Sequence
  Synthetically constructed DNA sequence to amplify genes from genomes or plasmids with various restriction sites to faciliate cloning
agtcagatctacaagagtccccgacga

SEQ ID NO: 161 - DNA, 48 bp, Artificial Sequence
  Synthetically constructed DNA sequence to amplify genes from genomes or plasmids with various restriction sites to faciliate cloning
catatggatagccggacatggtggatctgtcgacttgccaggattatg

SEQ ID NO: 162 - DNA, 26 bp, Artificial Sequence
  Synthetically constructed DNA sequence to amplify genes from genomes or plasmids with various restriction sites to faciliate cloning
atcgagatctatgggaatggaacatg

SEQ ID NO: 163 - DNA, 45 bp, Artificial Sequence
  Synthetically constructed DNA sequence to amplify genes from genomes or plasmids with various restriction sites to faciliate cloning
gatagccggacatggtggatctgtcgacccacaggtcagtcacgc

SEQ ID NO: 164 - DNA, 57 bp, Artificial Sequence
  Synthetically constructed DNA sequence to amplify genes from genomes or plasmids with various restriction sites to faciliate cloning
tcccatggagccagcatagtctgggacgtcatatggatagccggacatggtggatct

SEQ ID NO: 165 - DNA, 28 bp, Artificial Sequence
  Synthetically constructed DNA sequence to amplify genes from genomes or plasmids with various restriction sites to faciliate cloning
atcgagatctgataagtatgagaaagcg

SEQ ID NO: 166 - DNA, 53 bp, Artificial Sequence
  Synthetically constructed DNA sequence to amplify genes from genomes or plasmids with various restriction sites to faciliate cloning
catatggatagccggacatggtggatctgtcgaccgtttgcttgcttttccgg

SEQ ID NO: 167 - DNA, 52 bp, Artificial Sequence
  Synthetically constructed DNA sequence to amplify genes from genomes or plasmids with various restriction sites to faciliate cloning
gattacggctttcgagagtctgcagcagcagcagcaggcgaagatacctgcg

SEQ ID NO: 168 - DNA, 52 bp, Artificial Sequence
  Synthetically constructed DNA sequence to amplify genes from genomes or plasmids with various restriction sites to faciliate cloning
atgcagatctgataagtatgagaaagcggtagattacggctttcgagagtct

SEQ ID NO: 169 - DNA, 23 bp, Artificial Sequence
  Synthetically constructed DNA sequence to amplify genes from genomes or plasmids with various restriction sites to faciliate cloning
atgcagatcttttaacaataacc

SEQ ID NO: 170 - DNA, 47 bp, Artificial Sequence
  Synthetically constructed DNA sequence to amplify genes from genomes or plasmids with various restriction sites to faciliate cloning
catatggatagccggacatggtggatctgtcgacatctgcaacatgg

SEQ ID NO: 171 - DNA, 46 bp, Artificial Sequence
  Synthetically constructed DNA sequence to amplify genes from genomes or plasmids with various restriction sites to faciliate cloning
ggcttgaacctcaacaaggcagcagcagcagcaacacaagcacatg

SEQ ID NO: 172 - DNA, 35 bp, Artificial Sequence
  Synthetically constructed DNA sequence to amplify genes from genomes or plasmids with various restriction sites to faciliate cloning
gtagtaagaatgctaaaggcttgaacctcaacaag

SEQ ID NO: 173 - DNA, 46 bp, Artificial Sequence
  Synthetically constructed DNA sequence to amplify genes from genomes or plasmids with various restriction sites to faciliate cloning
atgcagatcttttaacaataacctgtgtagtaagaatgctaaaggc

SEQ ID NO: 174 - DNA, 28 bp, Artificial Sequence
  Synthetically constructed DNA sequence to amplify genes from genomes or plasmids with various restriction sites to faciliate cloning
atgcagatctagttacaacaagatcaac

SEQ ID NO: 175 - DNA, 47 bp, Artificial Sequence
  Synthetically constructed DNA sequence to amplify genes from genomes or plasmids with various restriction sites to faciliate cloning
catatggatagccggacatggtggatctgtcgacactatcatctaac

SEQ ID NO: 176 - DNA, 54 bp, Artificial Sequence
  Synthetically constructed DNA sequence to amplify genes from genomes or plasmids with various restriction sites to faciliate cloning
catctacttatacacacagtgcagcagcagcagcattggagttcatcacattgg

SEQ ID NO: 177 - DNA, 58 bp, Artificial Sequence
  Synthetically constructed DNA sequence to amplify genes from genomes or plasmids with various restriction sites to faciliate cloning
atgcagatctagttacaacaagatcaactcgtcatctacttatacacacagtgcagca

SEQ ID NO: 178 - DNA, 29 bp, Artificial Sequence
  Synthetically constructed DNA sequence to amplify genes from genomes or plasmids with various restriction sites to faciliate cloning
gcgtggatccctcacttgcgccacctccg

SEQ ID NO: 179 - DNA, 30 bp, Artificial Sequence
  Synthetically constructed DNA sequence to amplify genes from genomes or plasmids with various restriction sites to faciliate cloning
tcgagtcgactcagataatcatgatgctgt

SEQ ID NO: 180 - DNA, 27 bp, Artificial Sequence
  Synthetically constructed DNA sequence to amplify genes from genomes or plasmids with various restriction sites to faciliate cloning
gcgtggatccacaagagtccccgacga

SEQ ID NO: 181 - DNA, 29 bp, Artificial Sequence
  Synthetically constructed DNA sequence to amplify genes from genomes or plasmids with various restriction sites to faciliate cloning
tcgagtcgactcacttggcctcttgcgat

SEQ ID NO: 182 - DNA, 30 bp, Artificial Sequence
  Synthetically constructed DNA sequence to amplify genes from genomes or plasmids with various restriction sites to faciliate cloning
cagcggcggcggcgtctcaagctacgaacg

SEQ ID NO: 183 - DNA, 45 bp, Artificial Sequence
  Synthetically constructed DNA sequence to amplify genes from genomes or plasmids with various restriction sites to faciliate cloning
gttcgtagcttgagacgccgccgccgctgtcgaggttgccatgtc

SEQ ID NO: 184 - DNA, 40 bp, Artificial Sequence
  Synthetically constructed DNA sequence to amplify genes from genomes or plasmids with various restriction sites to faciliate cloning
ggctaacgtttcgattgcgaacgcgggatgaacccttcag

SEQ ID NO: 185 - DNA, 38 bp, Artificial Sequence
  Synthetically constructed DNA sequence to amplify genes from genomes or plasmids with various restriction sites to faciliate cloning
cgcaatcgaaacgttagccgccgccgccacgttcgtag

SEQ ID NO: 186 - DNA, 32 bp, Artificial Sequence
  Synthetically constructed DNA sequence to amplify genes from genomes or plasmids with various restriction sites to faciliate cloning
gcgtccatgggagctcacttgcgccacctccg

SEQ ID NO: 187 - DNA, 30 bp, Artificial Sequence
  Synthetically constructed DNA sequence to amplify genes from genomes or plasmids with various restriction sites to faciliate cloning
tcgaggtacctcagataatcatgatgctgt

SEQ ID NO: 188 - DNA, 52 bp, Artificial Sequence
  Synthetically constructed DNA sequence to amplify genes from genomes or plasmids with various restriction sites to faciliate cloning
agatcccggggggcagtgagatatgctcacttgcgccacctccgagcaacag

SEQ ID NO: 189 - DNA, 54 bp, Artificial Sequence
  Synthetically constructed DNA sequence to amplify genes from genomes or plasmids with various restriction sites to faciliate cloning
atcgccatgggaatggaacatgtaccagcagagttgaccagagtcagcgaaggg

SEQ ID NO: 190 - DNA, 58 bp, Artificial Sequence
  Synthetically constructed DNA sequence to amplify genes from genomes or plasmids with various restriction sites to faciliate cloning
accagagtcagcgaagggtatacacgattttaccggtccccaacggctagtgtaatac

SEQ ID NO: 191 - DNA, 59 bp, Artificial Sequence
  Synthetically constructed DNA sequence to amplify genes from genomes or plasmids with various restriction sites to faciliate cloning
caacggctagtgtaatactgtcaggattggtaaaggttaaatgggataatgaacaaatg

SEQ ID NO: 192 - DNA, 51 bp, Artificial Sequence
  Synthetically constructed DNA sequence to amplify genes from genomes or plasmids with various restriction sites to faciliate cloning
tgggataatgaacaaatgacgatgccgaccttcagcgtgactgacctgtgg

SEQ ID NO: 193 - DNA, 44 bp, Artificial Sequence
  Synthetically constructed DNA sequence to amplify genes from genomes or plasmids with various restriction sites to faciliate cloning
agatcccggggggcaatgagatatgatggaacatgtaccagcag

SEQ ID NO: 194 - DNA, 58 bp, Artificial Sequence
  Synthetically constructed DNA sequence to amplify genes from genomes or plasmids with various restriction sites to faciliate cloning
tcctgacagtattacactagccgttggggacgcagcggctgctgtatacccttcgctg

SEQ ID NO: 195 - DNA, 59 bp, Artificial Sequence
  Synthetically constructed DNA sequence to amplify genes from genomes or plasmids with various restriction sites to faciliate cloning
gtgtaatactgtcaggattggtaaaggttaaatgggctaatgcacaaatgacgatgccg

SEQ ID NO: 196 - DNA, 59 bp, Artificial Sequence; identical to SEQ ID NO: 61
  Synthetically constructed DNA sequence to amplify genes from genomes or plasmids with various restriction sites to faciliate cloning

SEQ ID NO: 197 - DNA, 34 bp, Artificial Sequence
  Synthetically constructed DNA sequence to amplify genes from genomes or plasmids with various restriction sites to faciliate cloning
atcgccatgggacacccagtttacgattacaatc

SEQ ID NO: 198 - DNA, 58 bp, Artificial Sequence
  Synthetically constructed DNA sequence to amplify genes from genomes or plasmids with various restriction sites to faciliate cloning
cacgccgttaaaaatgacaatcggttatttagattaatctttaaaactgacagcacag

SEQ ID NO: 199 - DNA, 52 bp, Artificial Sequence
  Synthetically constructed DNA sequence to amplify genes from genomes or plasmids with various restriction sites to faciliate cloning
ctttaaaactgacagcacagatattcaaaacaccttcagcgtgactgacctg

SEQ ID NO: 200 - DNA, 45 bp, Artificial Sequence
  Synthetically constructed DNA sequence to amplify genes from genomes or plasmids with various restriction sites to faciliate cloning
ccacgaggcgagctcggcacaactttttagcgcggcacgaatttc

SEQ ID NO: 201 - DNA, 50 bp, Artificial Sequence
  Synthetically constructed DNA sequence to amplify genes from genomes or plasmids with various restriction sites to faciliate cloning
ccgattgtcatttttaacggcgtgatagccccacgaggcgagctcggcac

SEQ ID NO: 202 - DNA, 46 bp, Artificial Sequence
  Synthetically constructed DNA sequence to amplify genes from genomes or plasmids with various restriction sites to faciliate cloning
atcgccatgggaaacaaccttggaacagtaccggatgtgccacatc

SEQ ID NO: 203 - DNA, 57 bp, Artificial Sequence
  Synthetically constructed DNA sequence to amplify genes from genomes or plasmids with various restriction sites to faciliate cloning
cagtaccggatgtgccacatcaaattccaaatgacaaaagtggtactcctgccattg

SEQ ID NO: 204 - DNA, 59 bp, Artificial Sequence
  Synthetically constructed DNA sequence to amplify genes from genomes or plasmids with various restriction sites to faciliate cloning
caaaagtggtactcctgccattgaagacccaaaagatatgaaaggattcaataaggctc

SEQ ID NO: 205 - DNA, 56 bp, Artificial Sequence
  Synthetically constructed DNA sequence to amplify genes from genomes or plasmids with various restriction sites to faciliate cloning
ccacaggtcagtcacgctgaaggttcgtacatttttatctttttctgccatgtatg

SEQ ID NO: 206 - DNA, 59 bp, Artificial Sequence
  Synthetically constructed DNA sequence to amplify genes from genomes or plasmids with various restriction sites to faciliate cloning
gacattgccggcggaaaaaggcgggcttatattgaacgcgctcatgaagaggacgatgc

SEQ ID NO: 207 - DNA, 53 bp, Artificial Sequence
  Synthetically constructed DNA sequence to amplify genes from genomes or plasmids with various restriction sites to faciliate cloning
gacattgccggcggacgtgccgcgctaaaaagtgctcatgaagaggacgatgc

SEQ ID NO: 208 - DNA, 49 bp, Artificial Sequence
  Synthetically constructed DNA sequence to amplify genes from genomes or plasmids with various restriction sites to faciliate cloning
ctttaaaactgacagcacagatattcaagctcatgaagaggacgatgcg

SEQ ID NO: 209 - DNA, 59 bp, Artificial Sequence
  Synthetically constructed DNA sequence to amplify genes from genomes or plasmids with various restriction sites to faciliate cloning
ctgtgctgtcagttttaaagattaatttaaataaccgtccgccggcaatgtcgccgttc

SEQ ID NO: 210 - DNA, 23 bp, Artificial Sequence
  Synthetically constructed DNA sequence to amplify genes from genomes or plasmids with various restriction sites to faciliate cloning
cattgccggcggaaagtttcttc

SEQ ID NO: 211 - DNA, 47 bp, Artificial Sequence
  Synthetically constructed DNA sequence to amplify genes from genomes or plasmids with various restriction sites to faciliate cloning
cgacattgccggcggacactttcttcgagctcatgaagaggacgatg

SEQ ID NO: 212 - DNA, 20 bp, Artificial Sequence
  Synthetically constructed DNA sequence to amplify genes from genomes or plasmids with various restriction sites to faciliate cloning
cattgccggcggacaatttc

SEQ ID NO: 213 - DNA, 47 bp, Artificial Sequence
  Synthetically constructed DNA sequence to amplify genes from genomes or plasmids with various restriction sites to faciliate cloning
cgacattgccggcggaagattttttcgagctcatgaagaggacgatg

SEQ ID NO: 214 - DNA, 47 bp, Artificial Sequence
  Synthetically constructed DNA sequence to amplify genes from genomes or plasmids with various restriction sites to faciliate cloning
cgacattgccggcggaagattttatcgagctcatgaagaggacgatg

SEQ ID NO: 215 - DNA, 47 bp, Artificial Sequence
  Synthetically constructed DNA sequence to amplify genes from genomes or plasmids with various restriction sites to faciliate cloning
cgacattgccggcggaagatttattcgagctcatgaagaggacgatg

SEQ ID NO: 216 - DNA, 47 bp, Artificial Sequence
  Synthetically constructed DNA sequence to amplify genes from genomes or plasmids with various restriction sites to faciliate cloning
cgacattgccggcggaagatttatgcgagctcatgaagaggacgatg

SEQ ID NO: 217 - DNA, 23 bp, Artificial Sequence
  Synthetically constructed DNA sequence to amplify genes from genomes or plasmids with various restriction sites to faciliate cloning
cattgccggcggaagatttgttc

SEQ ID NO: 218 - DNA, 24 bp, Artificial Sequence
  Synthetically constructed DNA sequence to amplify genes from genomes or plasmids with various restriction sites to faciliate cloning
cattgccggcggaagatttgctcg

SEQ ID NO: 219 - DNA, 27 bp, Artificial Sequence
  Synthetically constructed DNA sequence to amplify genes from genomes or plasmids with various restriction sites to faciliate cloning
cattgccggcggaagatttcttcaagc

SEQ ID NO: 220 - DNA, 47 bp, Artificial Sequence
  Synthetically constructed DNA sequence to amplify genes from genomes or plasmids with various restriction sites to faciliate cloning
cgacattgccggcggaagatttcttggagctcatgaagaggacgatg

SEQ ID NO: 221 - DNA, 47 bp, Artificial Sequence
  Synthetically constructed DNA sequence to amplify genes from genomes or plasmids with various restriction sites to faciliate cloning
cgacattgccggcggaagaatggttcgagctcatgaagaggacgatg

SEQ ID NO: 222 - DNA, 47 bp, Artificial Sequence
  Synthetically constructed DNA sequence to amplify genes from genomes or plasmids with various restriction sites to faciliate cloning
cgacattgccggcggaagacttggaacagctcatgaagaggacgatg

SEQ ID NO: 223 - DNA, 47 bp, Artificial Sequence
  Synthetically constructed DNA sequence to amplify genes from genomes or plasmids with various restriction sites to faciliate cloning
cgacattgccggcggaagacttactcaagctcatgaagaggacgatg

SEQ ID NO: 224 - protein, 390 aa, Magnaporthe grisea
MRGSHHHHHHGMASMTGGQQMGRDLYDDDDKDRWGSRSHPVYDYNPIPNHIHGDLKRRAYIERYSQCSDSQASEIRAALKSCAELASWGYHAVKNDNRLFKLIFKTDSTDIQNEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 225 - protein, 390 aa, Magnaporthe grisea
MRGSHHHHHHGMASMTGGQQMGRDLYDDDDKDRWGSRSHPVYDYNPIPNHIHGDLSASASIEAASACSDSQASEIASSAASCAELASWGYASAANDNAASALIFKTDSTDIQNEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 226 - protein, 363 aa, Puccinia striiformis
MRGSHHHHHHGMASMTGGQQMGRDLYDDDDKDRWGSRSMGTLLNVTRRLQNDGKPPDYCVDKWDEMMKERNKRLTGKPRGQCVDEIEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 227 - protein, 363 aa, Puccinia striiformis
MRGSHHHHHHGMASMTGGQQMGRDLYDDDDKDRWGSRSMGTLLNVTRRLQNDGKPPDYCVDKWDEMMKERNAAAAAKPRGQCVDEIEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 228 - protein, 413 aa, Melampsora lini
MRGSHHHHHHGMASMTGGQQMGRDLYDDDDKDRWGSRSNNLGTVPDVPHQIPNDKSGTPAIEDPKDMKGFNKALKSTPESEKLGTSSVEGIPQPEFDRGFLRPFGAKMKFLKPDQVQKLSTDDLITYMAEKDKNVREFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 229 - protein, 413 aa, Melampsora lini
MRGSHHHHHHGMASMTGGQQMGRDLYDDDDKDRWGSRSNNLGTVPDVPAAAANDAAAAPAIEDPAAAAGFNAAAASTPESEAAAASSVEGIPQPEFDAAAAAPFGAKMAAAAPDQVQAAAADDLITYMAEKDAAAAEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 230 - protein, 369 aa, Melampsora lini
MRGSHHHHHHGMASMTGGQQMGRDLYDDDDKDRWGSRSQYVVDPGFGEIECMCGQIARLTQRPFDVECEATPSCEFRSTMSGYPYDVPDYAGEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 231 - protein, 369 aa, Melampsora lini
MRGSHHHHHHGMASMTGGQQMGRDLYDDDDKDRWGSRSQYVVDPGFGEIECMCGQIAAAAARPFDVECEATPSCEFRSTMSGYPYDVPDYAGEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 232 - protein, 350 aa, Melampsora lini
MRGSHHHHHHGMASMTGGQQMGRDLYDDDDKDRWGSRSMGEFLEDARDIQGFSRKSGSKLEEESDSSRDRQEKEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 233 - protein, 350 aa, Melampsora lini
MRGSHHHHHHGMASMTGGQQMGRDLYDDDDKDRWGSRSMGEFLEDAASSAGFSRKSGSKLEEESDSSRDRQEKEFRSTMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 234 - DNA, 38 bp, Artificial Sequence
  Synthetically constructed DNA sequence to amplify genes from genomes or plasmids with various restriction sites to faciliate cloning
atgcagatctgaattcttagaggatgcacgagatatcc

SEQ ID NO: 235 - DNA, 63 bp, Artificial Sequence
  Synthetically constructed DNA sequence to amplify genes from genomes or plasmids with various restriction sites to faciliate cloning
ctgacagctcgcgcgataggcaagaaaaagtcgacagatccaccatgtccggctatccatatg

SEQ ID NO: 236 - DNA, 30 bp, Artificial Sequence
  Synthetically constructed DNA sequence to amplify genes from genomes or plasmids with various restriction sites to faciliate cloning
atgcagatctcagtatgttgttgatccagg

SEQ ID NO: 237 - DNA, 61 bp, Artificial Sequence
  Synthetically constructed DNA sequence to amplify genes from genomes or plasmids with various restriction sites to faciliate cloning
catatggatagccggacatggtggatctgtcgacacaacttggggtggcttcacattccac

SEQ ID NO: 238 - DNA, 28 bp, Artificial Sequence
  Synthetically constructed DNA sequence to amplify genes from genomes or plasmids with various restriction sites to faciliate cloning
atgcagatctactctgttgaacgtgact

SEQ ID NO: 239 - DNA, 51 bp, Artificial Sequence
  Synthetically constructed DNA sequence to amplify genes from genomes or plasmids with various restriction sites to faciliate cloning
catatggatagccggacatggtggatctgtcgacgttttgaatatctgtgc

SEQ ID NO: 240 - DNA, 37 bp, Artificial Sequence
  Synthetically constructed DNA sequence to amplify genes from genomes or plasmids with various restriction sites to faciliate cloning
atgcagatctatgggaaacaaccttggaacagtaccg

SEQ ID NO: 241 - DNA, 62 bp, Artificial Sequence
  Synthetically constructed DNA sequence to amplify genes from genomes or plasmids with various restriction sites to faciliate cloning
catatggatagccggacatggtggatctgtcgactcgtacatttttatctttttctgccatg

SEQ ID NO: 242 - DNA, 33 bp, Artificial Sequence
  Synthetically constructed DNA sequence to amplify genes from genomes or plasmids with various restriction sites to faciliate cloning
agtcagatctcacccagtttacgattacaatcc

SEQ ID NO: 243 - DNA, 51 bp, Artificial Sequence; identical to SEQ ID NO: 239
  Synthetically constructed DNA sequence to amplify genes from genomes or plasmids with various restriction sites to faciliate cloning

SEQ ID NO: 244 - DNA, 41 bp, Artificial Sequence
  Synthetically constructed DNA sequence to amplify genes from genomes or plasmids with various restriction sites to faciliate cloning
gaaacgcagcagctgcggcaaaacctcgagggcaatgtgtc

SEQ ID NO: 245 - DNA, 40 bp, Artificial Sequence
  Synthetically constructed DNA sequence to amplify genes from genomes or plasmids with various restriction sites to faciliate cloning
gttttgcagccgctgctgcgtttctctctttcatcatttc

SEQ ID NO: 246 - DNA, 36 bp, Artificial Sequence
  Synthetically constructed DNA sequence to amplify genes from genomes or plasmids with various restriction sites to faciliate cloning
catttgcagatgaagctggcacatccggtactgttc

SEQ ID NO: 247 - DNA, 48 bp, Artificial Sequence
  Synthetically constructed DNA sequence to amplify genes from genomes or plasmids with various restriction sites to faciliate cloning
cagcgctattgaatcctgctgaggcacttgggtcttcaatggcaggag

SEQ ID NO: 248 - DNA, 37 bp, Artificial Sequence
  Synthetically constructed DNA sequence to amplify genes from genomes or plasmids with various restriction sites to faciliate cloning
gacgaagccgcagcggcttcggattctggagtagatg

SEQ ID NO: 249 - DNA, 44 bp, Artificial Sequence
  Synthetically constructed DNA sequence to amplify genes from genomes or plasmids with various restriction sites to faciliate cloning
caaaaggcgcagaggctgcactgtcaaattctggttgagggatc

SEQ ID NO: 250 - DNA, 55 bp, Artificial Sequence
  Synthetically constructed DNA sequence to amplify genes from genomes or plasmids with various restriction sites to faciliate cloning
gatgcctgaacttggtccggactagcggctgacatttttgctccaaaaggcgcag

SEQ ID NO: 251 - DNA, 40 bp, Artificial Sequence
  Synthetically constructed DNA sequence to amplify genes from genomes or plasmids with various restriction sites to faciliate cloning
gtgccagcttcatctgcaaatgacaaaagtggtactcctg

SEQ ID NO: 252 - DNA, 48 bp, Artificial Sequence
  Synthetically constructed DNA sequence to amplify genes from genomes or plasmids with various restriction sites to faciliate cloning
cagcaggattcaatagcgctgccgcatctactccagaatccgaaaaac

SEQ ID NO: 253 - DNA, 37 bp, Artificial Sequence
  Synthetically constructed DNA sequence to amplify genes from genomes or plasmids with various restriction sites to faciliate cloning
gaagccgctgcggcttcgtcagttgaagggatccctc

SEQ ID NO: 254 - DNA, 34 bp, Artificial Sequence
  Synthetically constructed DNA sequence to amplify genes from genomes or plasmids with various restriction sites to faciliate cloning
cagtgcagcctctgcgccttttggagcaaaaatg

SEQ ID NO: 255 - DNA, 50 bp, Artificial Sequence
  Synthetically constructed DNA sequence to amplify genes from genomes or plasmids with various restriction sites to faciliate cloning
gtccggaccaagttcaggcatcttcagccgatgatctcatcacatacatg

SEQ ID NO: 256 - DNA, 51 bp, Artificial Sequence
  Synthetically constructed DNA sequence to amplify genes from genomes or plasmids with various restriction sites to faciliate cloning
atcgccatgggagaattcttagaggatgcagcgagcagcgcggggttctcc

SEQ ID NO: 257 - DNA, 42 bp, Artificial Sequence
  Synthetically constructed DNA sequence to amplify genes from genomes or plasmids with various restriction sites to faciliate cloning
caaatcgctgctgcggcagcacgaccatttgatgtggaatgt

SEQ ID NO: 258 - DNA, 36 bp, Artificial Sequence
  Synthetically constructed DNA sequence to amplify genes from genomes or plasmids with various restriction sites to faciliate cloning
tgctgccgcagcagcgatttgtccgcacatacattc

SEQ ID NO: 259 - protein, 24 aa, Phytophthora sojae
AMVESPDLVRRSLRNGDIAGGRFL

SEQ ID NO: 260 - protein, 24 aa, Phytophthora sojae
SLRNGDIAGGRFLRAHEEDDAGER

SEQ ID NO: 261 - protein, 117 aa, Phytophthora sojae; identical to SEQ ID NO: 19

SEQ ID NO: 262 - protein, 117 aa, Phytophthora sojae; identical to SEQ ID NO: 20

SEQ ID NO: 263 - protein, 49 aa, Phytophthora sojae
SENAFSAATDADQATVSKLAAAEFDTLVDVLTTESKRSLRATVDDGEER

SEQ ID NO: 264 - protein, 44 aa, Phytophthora sojae
TEYSDETNIAMVESPDLVRRSLRNGDIAGGRFLRAHEEDDAGER

SEQ ID NO: 265 - protein, 86 aa, Phytophthora sojae
LTCATSEQQTRPELCFFFSVRSSWPSTISDGACLALVSAEQGATAGRNTLSLRSMMATEDMATSTRSLRSQATNVDDDANVSIENR

SEQ ID NO: 266 - protein, 40 aa, Phytophthora sojae
LSTTNANQAKIIKGTSPGGHSPRLLRAYQPDDEGDSPEDR

SEQ ID NO: 267 - protein, 48 aa, Phytophthora sojae
VEPSATSTVEVAEVQARGADKRFLRSLQTEEEQGDSDVNEAEDGSEER

SEQ ID NO: 268 - protein, 50 aa, Phytophthora sojae
ITDESQPRDATIVDAPLTGRGANARYLRTSTSIIKAPDAQLPSTKAAIAS

SEQ ID NO: 269 - protein, 44 aa, Phytophthora sojae
TAEVDSKTALAAEVPAAIRSLESDTPASRLLRTGTVTSADNEDR

SEQ ID NO: 270 - protein, 38 aa, Phytophthora infestans
IDQTKVLVYGTPAHYIHDSAGRRLLRKNEENEETSEER

SEQ ID NO: 271 - protein, 33 aa, Phytophthora infestans
KADSLARTVSVVDNVKVKSRFLRAQTDEKNEER

SEQ ID NO: 272 - protein, 50 aa, Phytophthora infestans
AVSSNLNTAVNYASTSKIRFLSTEYNADEKRSLRGDYNNEVTKEPNTSDE

SEQ ID NO: 273 - protein, 38 aa, Phytophthora infestans
VAAFPIPDESRPLSKTSPDTVAPRSLRIEAQEVIQSGR

SEQ ID NO: 274 - protein, 44 aa, Hyaloperonospora arabidopsidis
TESSETSGTIVHVFPLRDVADHRNDALINRALRAQTALDDDEER

SEQ ID NO: 275 - protein, 48 aa, Hyaloperonospora arabidopsidis
LLHAHALHEDETGVTAGRQLRAAASEVFGLSRASFGLGKAQDPLDKFF

SEQ ID NO: 276 - protein, 92 aa, Leptosphaeria maculans
SPATKNNVNQPLDNISRRSEWKSVQISPVKEHSAKTADNTENNHNLEKRVFTSPHMKRTFTLALENTFYAMAWLIDFSFSEEGEPHFSYKLQ

SEQ ID NO: 277 - protein, 50 aa, Leptosphaeria maculans
CREASISGEIRYPQGTCPTKTEALNDCNKVTKGLIDFSQSHQRAWGIDMT

SEQ ID NO: 278 - protein, 48 aa, Leptosphaeria maculans
QPHLLCACESGRRDGVDDTRTLKVVKGTGGRFVFSSRYWTKAEGAPHE

SEQ ID NO: 279 - protein, 75 aa, Magnaporthe oryzae
HPVYDYNPIPNHIHGDLKRRAYIERYSQCSDSQASEIRAALKSCAELASWGYHAVKSDNRLFKLIFKTDSTDIQN

SEQ ID NO: 280 - protein, 50 aa, Magnaporthe oryzae
LPTPASLNGNTEVATISDVKLEARSDTTYHKCSKCGYGSDDSDAYFNHKC

SEQ ID NO: 281 - protein, 51 aa, Magnaporthe oryzae
RFCVYYDGHLPATRVLLMYVRIGTTATITARGHEFEVEAKDQNCKVILTNG

SEQ ID NO: 282 - protein, 47 aa, Magnaporthe oryzae
SFVQCNHHLLYNGRHWGTIRKKAGWAVRFYEEKPGQPKRLVAICKNA

SEQ ID NO: 283 - protein, 44 aa, Magnaporthe oryzae
ETGNKYIEKRAIDLSRERDPNFFDHPGIPVPECFWFMFKNNVRQ

SEQ ID NO: 284 - protein, 43 aa, Fusarium oxysporum
LPKGEEGDIIGTFNFSSSDSQPLKIHWVDTPDSSGSNLVKRSA

SEQ ID NO: 285 - protein, 81 aa, Fusarium oxysporum
LPVEDADSSVGQLQGRGNPYCVFPGRRTSSTSFTTSFSTEPLGYARMLHRDPPYERAGNSGLNHRIYERSRVGGLRTVIDV

SEQ ID NO: 286 - protein, 89 aa, Fusarium oxysporum
QEAAVREPQIFFNLTYTEYLDKVAASHGSPPDKSDLPWNDTMGSFPGNETDDGVQTETGSSLSRRGHIVNLRKREPFGEESRNDRVTQD

SEQ ID NO: 287 - protein, 50 aa, Fusarium oxysporum
NPAGDSLPDDAHLPDRRLSPSEVQALKKAQIYPPGYIHKRVTFGEGKDAV

SEQ ID NO: 288 - protein, 48 aa, Fusarium oxysporum
RDHQYCACQSGSGDSIDIDATTQLQNDNSKSYLWAQTSPAYWFADRHK

SEQ ID NO: 289 - protein, 48 aa, Fusarium oxysporum
GPLAQTESESADVAEHTINYIDIAPEEFEPPKANLSSLVSRDTLPVST

SEQ ID NO: 290 - protein, 50 aa, Melampsora lini
MEHVPAELTRVSEGYTRFYRSPTASVILSGLVKVKWDNEQMTMPLFKWIG

SEQ ID NO: 291 - protein, 100 aa, Melampsora lini
SLSNNLGTVPDVPHQIPNDKSGTPAIEDPKAAIEDPKDMKGFNKALKSTPESEKLGTSSVEGIPQPEFDRGFLRPFGAKMKFLKPDQVQKLSTDDLITYM

SEQ ID NO: 292 - protein, 33 aa, Melampsora lini
QYVVDPGFGEIECMCGQIARLTQRPFDVECEAT

SEQ ID NO: 293 - protein, 31 aa, Melampsora lini
EFLEDARDIQGFSRKSGSKLEEESDSSRDRQ

SEQ ID NO: 294 - protein, 71 aa, Ustilago maydis
NGSISNASHHHQRRMVRQRHIEARSAMSWLTKISSKASDWMFGSVHAPNLDKKDLPKPLVGGVAVMPKMPY

SEQ ID NO: 295 - protein, 47 aa, Laccaria bicolor
SPVPGEVGLVERGPIPNAVFRRVPEPNFFKDLLRALGQASQGGDLHR

SEQ ID NO: 296 - protein, 59 aa, Acyrthosiphon pisum
SAAEPYDEQEEASVELPMEHRQCDEYKSKIWDKAFSNQEAMQLMELTFNTGKELGSHEV

SEQ ID NO: 297 - protein, 29 aa, Mayetiola destructor
SPLPLAYTDQVYDACDRQFDETVRNSQPL

SEQ ID NO: 298 - protein, 154 aa, Mayetiola destructor
LVLDTRAMPETDFEKALKEWNRVQTLVLIAPEQRRTMVLIAEHLTNLKKMNVDSPGGSFLYLKDGDPVIKLPSVEHFEITFRGPYGVDKNFSFYMPKLKKLIVRDADANDKKIIKFVSQHSRTLKTLDLVAANYRTLRTLGAMKHIEEFVTSPP

SEQ ID NO: 299 - protein, 50 aa, Phytophthora sojae
TRVPDDANLQSVNAPVQTVTRSRRFLRTADTDIVYEPKVHNPGKKQVFIE

SEQ ID NO: 300 - protein, 101 aa, Pythium ultimum
MMPSTDAHGYIAFPPAQYKDPATATNYNAIITASINTAFAGKKWDDNPTANTKTFTAAFKKSGYTSLKQMLDKKVPGCQNSRTDFTPIKTKTYKTMEWQND

SEQ ID NO: 301 - protein, 99 aa, Pythium ultimum
HSQMTVPNPKFSDVSKANSPLGTIDGPTVMPPPAGQSYAMGTDTNIKAYVEAFAKQTKWKTLKDLIMDKYVEDGNIPDRACGLTDKTYMQPLPDKYVVW

SEQ ID NO: 302 - protein, 49 aa, Albugo candida
STMGLSNSRHLEDAVERVLGDLKLNQKDEKQNENEVDKNNSKGKDRESS

SEQ ID NO: 303 - protein, 54 aa, Aspergillus flavus
AGLPVFPNQAVLRPSLALPGDNSHRYSLPMFDLQPWERVDEIRLARKGYLYGSP

SEQ ID NO: 304 - protein, 43 aa, Pyrenophora triticirepentis
QVKGNAIRCGQDDKSDQDTRNFCRFMFTSDRTLKINGEFRGNA

SEQ ID NO: 305 - protein, 49 aa, Alternaria brassicicola
APASYPASALGKRWVDTTGGQKMPAHFVSTVRKLSTEKLKTKRQLDQLL

SEQ ID NO: 306 - protein, 51 aa, Cryptococcus neoformans
LTVPQAHRETLEEAGKLTTIAAINTKKLIKDVTVGTMSSVFPDGTDNGGRP

SEQ ID NO: 307 - protein, 61 aa, Cryptococcus neoformans
IQQGQKANAREQHRGRKTNLTIKLPGAHSYKAKFEGCMVVLQDKKLYVEHAGCESLAYAHP

SEQ ID NO: 308 - protein, 94 aa, Aspergillus fumigatus
IPSAFPQDNAVNQVLLSDSYQDQSVSSISAEDDAQNSAVIHIGESETMRAPSWFTSTLMARRLLALSTTGTVSTIFPDPLPGNSHAPPSVAGLP

SEQ ID NO: 309 - protein, 77 aa, Aspergillus fumigatus
VAMGVSEQRKANERKMDARRMARFNIDIETSGETQEEDEIRGKRIVLRDNKVYLDDPLPANRKHPSHTAESFYIDYP

SEQ ID NO: 310 - protein, 49 aa, Aspergillus fumigatus
PVVPGQTVMEPSAALPDDGDHLYTLPMFDIRPWERVSEVRLAREGYLYG

SEQ ID NO: 311 - protein, 89 aa, Coccidioides immitus
SPVFPGGDKRDALYQKPIAPAGEFPFDSSPPEARMTIPYADNEPDSSLSIPSWPTTHLLARRLLGLSTTGVLSTVFPRTNRDPALVGVP

SEQ ID NO: 312 - protein, 76 aa, Coccidioides immitus
IRSSQRSQRRQEHRSRKMNLIVSCSDPSRKSKDVDGCFVVLRNHKLWIASRPSDGEANEPSDDATRFKASLHQCHH

(19) United States
(12) Patent Application Publication
(10) Pub. No.: US 2011/0212541 A1
(43) Pub. Date: Sep. 1, 2011

(54) COMPOSITIONS AND METHODS TO PROTECT CELLS BY BLOCKING ENTRY OF PATHOGEN PROTEINS

(75) Inventors: Brett Tyler, Blacksburg, VA (US); Shiv Kale, Fairfax, VA (US); Vincenzo Antignani, Blacksburg, VA (US)

(73) Assignee: Virginia Tech Intellectual Properties, Inc., Blacksburg, VA (US)

(21) Appl. No.: 12/944,345

(22) Filed: Nov. 11, 2010

Related U.S. Application Data
(63) Continuation-in-part of application No. 12/468,470, filed on May 19, 2009.
Application No. 12/468,470 is a continuation-in-part of application No. PCT/US2009/044489, filed on May 19, 2009.
(60) Provisional application No. 61/260,227, filed on Nov. 11, 2009.
(60) Provisional application No. 61/128,080, filed on May 19, 2008.
(60) Provisional application No. 61/160,059, filed on Mar. 13, 2009.

(51) Int. Cl. G01N 33/53; C12N 5/04; C12N 5/07; C12N 5/071; C12N 5/077; C12N 5/078; C12N 5/079

(52) U.S. Cl. 436/501; 435/410; 435/412; 435/414; 435/415; 435/417; 435/416; 435/325; 435/350; 435/351; 435/366; 435/368; 435/369; 435/370; 435/371; 435/372

(57) ABSTRACT

Pathogenic effector proteins include one or more virulence motifs of amino acid consensus sequence BXZ, where B=RK or H; X=any amino acid or is absent; Z=L, M, I, W, Y or F) which bind to target polar lipids on a host (plant or animal) cell as a prerequisite for translocation of the pathogenic effector proteins into the cell. Translocation is prevented by binding blocking compounds to one or more motifs of the effector protein or to the lipid ligands of the host cell. The blocking compounds include synthetic or naturally occurring polypeptides which bind the polar lipids or the motifs, various polar lipids, the hydrophilic head-groups of polar lipids, etc. Suitable blocking compounds can be identified by assays demonstrating binding to the motifs or to the target polar lipids.

### CROSS-REFERENCE TO RELATED APPLICATIONS

This application claims priority to U.S. Provisional Patent Application Ser. No. 61/128,080, filed May 19, 2008; Ser. No. 61/160,059, filed Mar. 13, 2009; and Ser. No. 61/260,227, filed Nov. 11, 2009. This application is a continuation-in-part of U.S. patent application Ser. No. 12/468,470 filed May 19, 2009; and of International patent application PCT/US09/044,489, filed May 19, 2009. The complete contents of each of these applications are herein incorporated by reference.

### STATEMENT OF GOVERNMENT INTEREST

This invention was made with funding under USDA/CSREES/NRICGP Grant No. 2007-35319-18100, and the United States government has certain rights in the invention.

### SEQUENCE LISTING

This application includes as the Sequence Listing the complete contents of the accompanying text file “Sequence.txt”, created Nov. 10, 2010, containing 307,236 bytes, hereby incorporated by reference.

### BACKGROUND OF THE INVENTION

1. Field of the Invention

The present invention generally relates to prevention of microbial, especially oomycete or fungal, disease and, more particularly, to cellular targets for blocking entry of pathogen effector proteins into plant or animal cells. The invention also provides compositions and methods for identifying compounds that block entry of pathogen effector proteins into cells, and treatments using such compounds.

2. Background Description

Fungi and parasites such as Plasmodium are eukaryotes, which are organisms that have complex internal cell structures (bacteria and viruses have simpler structures and are excluded). Infections by parasites and fungi are especially difficult to develop drugs for because humans are also eukaryotes, so many drugs toxic to these organisms are also toxic to humans. In addition to the malaria parasite, Plasmodium, other eukaryotic pathogens of humans include the parasites Schistosoma, Onchocerca, Trypanosoma, and Leishmania, fungi that afflict AIDS patients such as Candida, Histoplasma, Cryptococcus and Aspergillus, and the Valley Fever fungus, Coccidioides, that affects healthy people in the Southwest. Fungal spores are also responsible for allergies, asthma and mold-related illnesses.

Eukaryotic pathogens of plants are also a major problem in agriculture, horticulture and forestry, and include fungi and fungal-like organisms related to marine algae called oomycetes. These diseases cause billions of dollars in losses each year. Some fungal plant pathogens include rust fungi, such as the new virulent wheat rust fungus, Ug99, that is sweeping through Africa and the middle east, and the rice blast fungus which causes major losses to the US and Asian rice crop each year. Oomycete pathogens include the late blight pathogen of potato (Phytophthora infestans) that causes the Irish potato famine and still causes $5 billion in losses worldwide annually, Phytophthora ramorum that causes Sudden Oak Death in California, and Phytophthora sojae that caused $1-2b damage to the US soybean crop. Worldwide transport of plants and plant products across diverse ecosystems has hastened the spread of many plant pathogens. With the increased pressure on agricultural production systems due to competing needs for food and biofuels, there is an urgent need to explore new highly efficacious strategies for biotechnology-based approaches to disease control.

Eukaryotic pathogens of both humans and plants release protein toxins called effectors that have the ability to infiltrate inside host cells, across the membrane barrier that normally surrounds the host cells. Once the effectors enter the host cell, they reprogram the cells to suppress or block the immune responses of the host and to make the host tissue more congenial for reproduction and spread of the pathogen. Therefore, drugs that could block the entry of effector proteins into host cells would potentially suppress infection by a broad range of eukaryotic pathogens important to medicine and agriculture.

Like animals, plants have evolved defense mechanisms that afford some protection from pathogens. Constitutive defenses include structures such as the cuticle and preformed anti-microbial chemicals. Plants have also evolved an active defense response that is induced by detection of an attacking pathogen. The response includes rapid synthesis of anti-microbial chemicals and proteins, and a programmed cell death (PCD) response, called the hypersensitive response (HR). The ability of plants to detect and respond to pathogens is mediated by various receptors and signal transduction pathways that have close similarities to the innate immunity mechanisms of animals. Unfortunately, however, pathogens of both plants and animals have evolved mechanisms to avoid or suppress host defenses, thereby retaining the ability to cause many destructive diseases affecting crops and forests.

Oomycetes are fungus-like organisms many of which are pathogens. For example, most of the more than 80 species of the oomycete genus Phytophthora are destructive pathogens, including the potato late blight pathogen, Phytophthora infestans, which caused the Irish potato famine in the 18th century, the soybean root and stem rot pathogen P. sojae, and Phytophthora ramorum, the causative agent of Sudden Oak Death that is currently ravishing oak forests in California. The closely related oomycete genus Pythium contains more than 100 species, most of which are also pathogens. The oomycetes also include a number of commercially important and diverse downy mildew pathogens that are obligate parasites, often with narrow host ranges.

The sequencing of Expressed Sequence Tags (ESTs) and genomes from several oomycete pathogens has been completed or is under way. Draft genome sequences for the soybean pathogen Phytophthora sojae and for P. ramorum have been completed; those of P. infestans and the Arabidopsis downy mildew pathogen Hyaloperonospora arabidopsidis are nearing completion; and genome sequencing of the broad host range plant pathogens Phytophthora capsici and Pythium ultimum and the fish pathogen Saprolegnia parasitica is also underway. In addition, substantial libraries of EST sequences are available for most of these species, as well as for Phytophthora parasitica and the Saprolegniomycete plant pathogens Aphanomyces euteiches and Aphanomyces cochlioides. The mining of these pathogen sequences by comparative genomics and the prediction of which proteins are secreted by the pathogens has resulted in identification of large numbers of candidate genes that potentially encode proteins involved in plant infection.

Among these are the so-called “effector proteins” or “effectors”. Effectors, which are secreted by plant pathogens and have the ability to enter plant cells, have been documented for many classes of plant pathogens, including bacteria, fungi, oomycetes and nematodes. Once inside a host cell, the major function of an effector protein is to suppress the signal transduction pathways that mediate plants defense responses, and many effector proteins also suppress host programmed cell death. The activities of fungal effector proteins are known to include chitin-binding, cytotoxicity, metalloprotease activity, and protease inhibition. Pathogen effectors may also reprogram the plant cell to promote nutrition of the pathogen.

In response to pathogen attacks mediated by effectors, plants have evolved certain resistance (“R”) genes that encode receptor proteins having the ability to bind and sequester, and thereby inactivate, the pathogen effector proteins. (In fact, effectors were initially discovered based on their ability to trigger responses mediated by R gene-encoded host receptors). Pathogen genes encoding effectors are referred to as avirulence (Avr) genes, because, in practice, they actually prevent infection of host plants which contain cognate receptor proteins by binding to the receptor, thereby alerting the plant to their presence, and initiating an anti-pathogen response. In contrast, genes encoding plant effectors for which cognate plant receptors do not exist are referred to as virulence genes.

Genetic mapping of oomycete Avr genes led to the cloning of the first four effector genes: Avr1b-1 from P. sojae (Shan, W., Cao, M., Leung, D. & Tyler, B. M. The Avr1b locus of Phytophthora sojae encodes an elicitor and a regulator required for avirulence on soybean plants carrying resistance gene Rps1b. Mol. Plant. Microbe Interact 17, 394-403 (2004); Avr3a from P. infestans (Armstrong, M. R. et al. An ancestral oomycete locus contains late blight avirulence gene Avr3a, encoding a protein that is recognized in the host cytoplasm. Proc Natl Acad Sci USA 102, 7766-71 (2005); and ATR1 (Rehmany, A. P., Gordon, A., Rose, L. E., Allen, R. L., Armstrong, M. R., Whisson, S. C., Kamoun, S., Tyler, B. M., Birch, P. R., and Beynon, J. L. (2005). Differential recognition of highly divergent downy mildew avirulence gene alleles by RPP1 resistance genes from two Arabidopsis lines. Plant Cell 17, 1839-1850), and ATR13 (Allen, R. L., Bittner-Eddy, P. D., Grenville-Briggs, L. J., Meitz, J. C., Rehmany, A. P., Rose, L. E., and Beynon, J. L. (2004). Host-parasite coevolutionary conflict between Arabidopsis and downy mildew. Science 306, 1957-1960), both from H. arabidopsidis. Many other effector genes have subsequently been identified and analyses have shown that all effector genes encode small secreted hydrophilic proteins that lack disulfide bonds. Significantly, effector proteins have the ability to enter plant cells unaided by any other pathogen encoded molecules. Thus, the mechanism of entry must lie in the effector proteins themselves. Sequence comparisons have led to the identification of two common motifs in the N-terminus region of effector proteins: 1) RxLR or RXLR (which stands for “arginine, any amino acid, leucine, arginine”); and 2) dEER (which stands for “aspartate which is not highly conserved, glutamate, glutamate, arginine”) (Birch, P. R., Rehmany, A. P., Pritchard, L., Kamoun, S., and Beynon, J. L. 2006. Trends Microbiol 14, 8-11; Tyler, 2006. Science 313, 1261-1266; Rehmany, et al. 2005. Plant Cell 17, 1839-1850). These motifs have been suspected of being responsible for the ability of effector proteins to enter plant cells. This speculation has been encouraged by the observation that a similar N-terminal “Pexel” motif (RxLxE/Q, i.e. “arginine, any amino acid, leucine, any amino acid, then aspartate or glutamate or glutamine”) is required for effectors of the malaria parasite, Plasmodium, to cross the host parasitiphorous vacuolar membrane into the cytoplasm of red blood cells. In addition, experimental evidence has shown that mutations in either the RxLR or dEER motifs can alter an effector's ability to translocate into host cells.

Effectors of fungal plant pathogens have also been predicted to translocate into host cells because many plants (e.g. flax and rice) possess intracellular receptors, encoded by major resistance (R) genes, which mediate a rapid defense response when fungal effectors are present. However, prior to the present invention, fungal effectors had not been well characterized and the presence of amino acid sequence motifs that mediated entry into host cells had not been demonstrated.

In spite of previous suspicions concerning putative involvement of the RxLR and dEER motifs in effector translocation, the precise mode of and requirements for translocation of effector proteins were not known. And, in fact, it was previously not known whether fungal pathogens even possessed these or analogous motifs. This lack of knowledge had hindered the development of effective methods to combat the infection of both plant and animal host cells by oornycete, fungal and Plasmodium pathogens. Further, the lack of detailed characterization of the RxLR and dEER motifs and their flanking sequences has prevented the selection, from an enormous pool of genomic sequence data, of genes that likely encode additional effector proteins, the identification of which could lead to strategies for inhibiting their pathogenic action in cells.

### SUMMARY OF THE INVENTION

The present invention provides methods to block the entry of pathogen effector proteins into host cells (e.g., “translocation”), thereby preventing host cell infection. The methods are based on the discovery that binding of polar lipids such as phosphatidyl-inositol-3-phosphate (PI-3-P) and/or phosphatidyl-inositol-4-phosphate (PI-4-P) and/or phosphatidic acid to effector molecules via a virulence motif is a prerequisite to translocation of the effector into a host cell, and that when binding is blocked, translocation, and hence infection of the cell, does not occur. The motifs have the sequence “BXZ” where B=arginine, lysine or histidine; X=any amino acid or no amino acid (i.e. X may be absent); and Z=leucine, methionine, isoleucine, tryptophan, tyrosine or phenylalanine. The BXZ motif encompasses a family of motifs, which frequently occur at or near the N-terminus of an effector protein, examples of which include but are not limited to RxLR (which may function in concert with a dEER motif), Pexel, RYWT, RIYER, RSLR, RRLLR, RRFLR, and RFYR, and others, all of which may collectively be referred to herein as “virulence motifs.

Binding may be prevented by any of several strategies including but not limited to i) blocking the effector motif itself, and ii) blocking the lipids of the cell to which the motif binds. The motif itself may be blocked by e.g. inositol 1,4-diphosphate, or by other inositol containing phosphatidic acids, phospholipids and sphingolipids, or any other compound which binds to one or the virulence motif. Blocking of the lipid (generally at or on the cell surface) can be accomplished using, for example, proteins or peptides or other molecules that bind the lipids (e.g. mimetics of the motifs, or proteins or other compounds that destroy, remove or block access to phosphatidyl-inositol-3-phosphate (PI-3-P) and/or phosphatidyl-inositol-4-phosphate (PI-4-P) and/or phosphatidic acid and/or any other polar lipid that is bound by an effector) thereby preventing effector binding to a motif. According to the invention, the entry of effector proteins from oomycetes, fungi, and other types of pathogens, including human pathogens (e.g. Plasmodium) may be blocked. For example, Plasmodium effector proteins include a Pexel motif which is selectively bound as a prerequisite for translocation. Blocking of effector entry prevents the pathogen from inhibiting host cell defense mechanisms and allows the host to mount an effective response to the pathogen.

The invention also provides elucidation of the structural requirements of the virulence motifs in oomycetes and fungi, and of the sequences which flank the motifs, leading to the ability to predict which genes in the genome of a pathogen are likely to encode effector molecules.

According to an embodiment the invention, translocation of an effector protein from a pathogen, such as a bacteria, fungus, oomycete, protozoa or nematode, into a host including animals (including humans) and plants is prevented by selectively binding a blocking compound to one or more BXZ virulence motifs of the effector protein. According to another embodiment, translocation of an effector protein from a pathogen is prevented by selectively binding a blocking compound to one or more polar lipids which would otherwise bind an effector motif. By preventing entry of the effector protein into the cell, the host cell defense mechanisms are permitted to mount an effective defense against the pathogen (it being recognized that after entry, the effector protein would compromise the host cell defense mechanisms). Thus, the invention provides a mechanism to avoid the adverse outcomes attributed to pathogenic effector proteins, and it is applicable in promoting the health and viability of both plants and animals.

Another embodiment of the invention pertains to identifying compounds which are suitable for use in protecting cells (animal and plant) from pathogenic effector proteins. In operation, an assay is used to determine whether or not a compound binds to one or more motifs of an effector protein which are bound by phosphoinositides or another polar lipid as a prerequisite for translocation. The assay may include pathogenic effector proteins which include a BXZ motif (e.g. RxLRPexel, RYWT, RIYER, RSLR, RRLLR, RRFLR, and RFYR or similar motifs), and/or may include protein substrates which present one or more BXZ motifs in a manner which can be bound by a candidate compound. Alternatively, an assay may include one or more polar lipids which bind to the motifs, in order to identify compounds which bind and thus would block motif binding. Such assays may include competition assays between candidate compounds and compounds (e.g. peptides or proteins) which contain the motif(s). The assay may be in the solid or liquid phase and may employ fluorescent, phosphorescent, chemiluminescent, colorimetric, or other suitable labels to indicate binding of a candidate compound to one or more motifs which are required to be bound by phosphoinositides or another polar lipid as a prerequisite for translocation.

Yet another embodiment of the invention pertains to a methodology of identifying whether an amino acid sequence of a protein in a pathogen is part of an effector protein. In this embodiment, hidden Markov modeling (HMM) is used to compare flanking sequences of a BXZ sequence (e.g. of an RxLR sequence) to determine whether the structural features for the motif are present. Through identification of effector proteins, effective strategies for preventing entry of the effector proteins into a cell (animal or plant) can be pursued.

### BRIEF DESCRIPTION OF THE DRAWINGS

FIG. 1A-E. RxLR and dEER motifs are required for Avr1b function in P. sojae transformants. (A) Sequences of mutations in the RxLR1, RxLR2 and dEER motifs. Bold indicates amino acids of the RxLR motifs and the alanines used to replace them in the mutations. Italics indicate the dEER motif and the alanines used to replace it in the mutant. (B) Pst I restriction analysis of PCR products amplified from Avr1b-1 transformants using primers specific for the HAM34 promoter and terminator regions. Pst I restriction profiles of Avr1b(RxLR1^{AAAA}), Avr1b(RxLR2^{AAAA}), Avr1b(RxLR1^{AAAA}, 2^{AAAA}), Avr1b(dEER^{A6}) and wild type (WT) Avr1b are distinguished from each other because the mutations introduce a Pst I site. Avr1b(dEER^{A6})-9 was confirmed by sequencing the PCR product. C, Detection of Avr1b mRNA in P. sojae stable transformants by RT-PCR. Upper panel shows amplification with primers internal to the Avr1b C-terminus. Lower panel shows amplification with P. sojae actin primers. P. sojae stable transformants were the same as for (B) except that an amplification reaction is also shown from RNA from a P. sojae transformant containing a β-glucuronidase gene (GUS). No amplification was observed when reverse transcriptase was omitted from the reactions. (D) Distributions of HMM scores of RxLR flanking regions for all RxLR-containing secreted proteins from P. sojae and P. ramorum (non-permuted), for all secreted proteins retaining an RxLR string after sequence permutation (permuted), and for all high quality RxLR-effector candidates identified by Jiang et al (2008) (curated). The locations on the distribution of the HMM scores of the RxLR strings of known avirulence proteins and HpAvh341 are shown by the arrows. (E) Phenotype of L77-1863 (Rps1b) seedlings inoculated on the hypocotyls with transformants carrying the indicated wild type or mutant Avr1b-1 genes and photographed 4 days later.

FIG. 2. RxLR and dEER functions confirmed by particle bombardment assay. Soybean leaves were bombarded using a double-barreled device that delivered Avr1b-1 DNA-bearing particles to one side of the leaf and control (empty vector) DNA to the other; both sides received GUS DNA. Ratio of blue spots in the presence of Avr1b-1 compared to the control. sAvr1b indicates a gene encoding secretory Avr1b and mAvr1b indicates one encoding mature Avr1b (lacking the secretory leader). WT indicates wild-type RxLR motif, RxLR2^{AAAA} indicates the four alanine replacement of the RxLR2 motif, dEERA6 indicates the six alanine replacement of the dEER motif. Averages and standard errors are from 16 pairs of shots. p values comparing results from cultivars with Rps1b (L77-1863) or without (rps; Williams) were calculated using the Wilcoxon rank sum test.

FIG. 3A-D. P. sojae stable transformants show that two other Avh proteins can replace the RxLR and dEER region of Avr1b. (A) Sequences of the N-termini of wild type and mutant Avr1b proteins, and of fusions with two other Avh proteins. Underlined, secretory leader; bold, RxLR motifs; italics, dEER motifs. The C-terminal sequence of Avr1b is shown in lowercase. (B) PCR analysis of DNA from P. sojae stable transformants. WT: p1=pHamAvr1b plasmid DNA, T17 and T20=two transformants with wild type Avr1b-1 transgenes. HpAvh341-Avr1bCt: p1=pHamAvh341 plasmid DNA (encoding Hp Avh341-Avr1bCt), 13 and 17=two transformants containing pHamAvh341. PsAvr4/6-Avr1bCt: pHamAvh171 plasmid DNA (encoding Ps Avr4/6-Avr1bCt), 3 and 19=two transformants containing pHamAvh171. mAvr1bCt: p1=pHamAvr1bCt plasmid DNA (encoding mAvr1bCt protein), 4 and 5=two transformants containing pHamAvr1bCt. The sizes of the PCR products for Avr1b-1, pHamAvh341, pHamAvh171 and pHamAvr1bCt are 577 bp, 721 bp, 748 bp and 385 bp respectively C, Detection of Avr1b mRNA in P. sojae stable transformants by RT-PCR. Upper panel shows amplification with primers internal to the Avr1b C-terminus. Lower panel shows amplification with P. sojae actin primers. P. sojae stable transformants were the same as for (B) except that an amplification reaction is also shown from RNA from a P. sojae transformant containing a β-glucuronidase gene (GUS). p1=pHamAvr1b plasmid DNA as template. No amplification was observed when reverse transcriptase was omitted from the reactions (D) Phenotype of L77-1863 (Rps1b) seedlings inoculated on the hypocotyls with the indicated transformants carrying wild type or mutant Avr1b-1 genes and photographed 4 days later. HpAvh341-Avr1b-17, PsAvr4/6-Avr1b-3 and mAvr1bCt-5 gave similar results to HpAvh341-Avr1b-13, PsAvr4/6-Avr1b-19 and mAvr1bCt-4 (Table 1).

FIGS. 4A and B. Functional replacement of Avr1b host targeting signal with protein transduction motifs and Plasmodium host targeting signals. (A) Sequences of modified Avr1b proteins. PfGBP, PfHRP and Pf1615c refer to the Plasmodium Pf GBP-130, Pf HRPII and Pf PFE1615c proteins. All non-native Avr1b sequences are underlined, Avr1b RxLR2 and Plasmodium RxLXE/Q motifs are in bold, and acidic residues in the dEER region are in italics. The Avr1b secretory leader was used in all constructs. (B) Ratio of blue spots in the presence of Avr1b-1 compared to the control, assayed as described in FIG. 2. Constructs are as in (A). Averages and standard errors are from 8 pairs of shots.

FIG. 5. Summary of Avr1b-1 mutations and their phenotypes in P. sojae stable transformants and soybean transient expression assays. A, Avirulent; V, Virulent; NT, Not Tested; Y, significantly fewer blue (GUS-positive) tissue patches from GUS expression resulting from Avr1b-induced cell death; N, not significantly fewer blue tissue patches; P, partial reduction in blue tissue patches; SP, signal peptide.

FIGS. 6A-H. Binding of oomycete effector proteins to phosphoinositides. a-c, Filter-binding assays. d-f, Liposome binding assays. RxLR and dEER mutations are described in FIGS. 6G and H. (N)-GFP indicates a fusion of the N-terminal domain to GFP. (FL)-GST indicates a fusion of the full length effector proteins (without signal peptide) to GST. In d-f, B and F indicate liposome-bound and -free proteins respectively; M=size markers. PI-3-P=phosphatidylinositol-3-phosphate; PI-4-P=phosphatidyl inositol-4-phosphate; PI-5-P=phosphatidyl inositol-5-phosphate; PI=phosphatidyl inositol; PA=phosphatidic acid; PS=phosphatidyl serine; PE=phosphatidyl ethanolamine; PC=phosphatidyl choline. No mutant proteins bound to PI-5-P, PT, PA, PS, PE or PC (not shown).

FIGS. 7A-C. Identification of host-targeting signals in fungal effectors a, Particle bombardment cell re-entry assays of fungal effectors fused to Avr1b. N-terminal sequences of AvrL567, AvrM and AvrPi-ta (shown in b) were fused to the secretory leader (s) and C-terminal domain of Avr1b. AvrL567-Avr1b fusions lacking the secretory leader (m) or with mutations in the putative RxLR and dEER motif (rfyr-de-) were also assayed. Effector re-entry resulting in cell killing was measured by double-barreled particle bombardment in which parallel bombardments with a beta-glucuronidase (GUS) reporter gene, with and without the Avr1b fusion, were compared in the presence of resistance gene Rps1b (cultivar L77-1863) or in its absence (rps; cultivar Williams). Averages and standard errors shown are from 14-16 pairs of bombardments. P values were calculated using the Wilcoxon rank sum test. b, N-terminal sequences of effectors tested in a, with RXLR-like motifs shaded and dEER-like motifs underlined. The start of the Avr1b C-terminal domain used for all fusions is boxed. Three sequences containing potential motifs from AvrPi-ta and one from AvrL567 were inserted into Avr1b in place of the RFLR motif. Cell entry activity of each sequence is given as relative ablation. Ablation=[1−(GUS+spots on Rps1b)/(GUS+spots on rps)]. Relative ablation=ablation of construct/ablation of wild-type Avr1b. In the sequence alignment, dashes indicate identical residues, periods indicate gaps in the alignment and * * * * indicates Avr1b sequences.

FIGS. 8A-F. Binding of P. falciparum effector fusion proteins to phosphoinositides a-c, Filter binding assays Mutant proteins did not bind to PI-5-P, PI, PA, PS, PE or PC (not shown) except PfGBP(N)-GFP(pexel-) which bound weakly to PS. d-f, Binding of wild-type and mutant fusion proteins to PT-3-P or PI-4-P in liposomes; B and F indicate liposome-bound and -free proteins respectively; M=size markers.

FIGS. 9A-D. Modulation of effector entry into root cells by phosphoinositides a, Stimulation of Avr1b(N)-GFP entry by PI-4-P and inhibition by IP2. b, Stimulation of AvrL567(N)-GFP entry by PI-4-P and inhibition by IP2. c, Entry of Arg9-GFP is not stimulated by PI-4-P nor inhibited by IP2. In each case, 1 mg/ml protein was incubated with soybean root tips for 9 hr or 12 hr then washed and photographed. Either 250 μM di-octanoyl-PI-4-P or 500 μM IP2 was preincubated with the proteins for 30 min prior to exposure to the roots. Paired light micrographs and fluorescence optical sections are from the same root tips in each case. Lighting and photographic exposure were identical for all photographs. d, Inhibition of effector-binding to liposomes by IP2. Binding of Avr1b(N)-GFP, AvrL567(N)-GFP and Arg9-GFP to liposomes containing PI-4-P was measured in the presence or absence of 300 μM inositol 1,4 diphosphate (1,4IP2). Also, binding of Arg9-GFP to liposomes containing PI-3-P was measured in the presence or absence of 300 μM inositol 1,3 diphosphate (1,3IP2).

FIGS. 10A-E. Effector entry into human cells and inhibition by inositol diphosphates a-d, Cells of the human lung epithelial cell line A549 were incubated with the indicated fusion proteins (1 mg/ml) for 15 hr, in the presence or absence of 430 μM inositol 1,3 diphosphate (1,3IP2), 440 μM inositol 1,4 diphosphate (1,4IP2) or 240 μM dioctanoyl-PI-4-P, then washed and photographed. Paired light micrographs and fluorescence optical sections are from the same cells in each case. Lighting and photographic exposure were identical for all photographs.

FIGS. 11A-B. Description of plasmids used in Example 1.

FIGS. 12A-B. Oligonucleotides used for plasmid construction. Uppercase letters indicate bases that match the initial template. Lower case letters indicate mutations or 5′ extensions that do not match the initial template. Restriction sites introduced into the amplicon are underlined. A pipe (|) indicates the boundary between Avr1b-1 sequences and fused sequences (Avh, GFP or Plasmodium RXLX motif) in the fusion oligonucleotides.

FIGS. 13 A-F. Description of plasmids used in Example 2.

FIG. 14A-F. Oligonucleotides used. Restriction sites are in bold. Mutations created by the primers are in lower case.

FIG. 15. Binding of fungal effector proteins to phosphatidic acid shown in tabular form. Filter-binding assay were used to test which polar lipids were bound bind by the indicated fungal effector proteins. The N-terminus of each fungal effector (documented in the sequence list) was fused to GFP, the fusion proteins were purified from E. coli, and then tested for binding to the same polar lipids as documented in FIG. 6. Mutant proteins contained amino acid substitutions in the motifs listed in the row above (A=alanine; S=serine). All the wild-type proteins listed bound phosphatidic acid, but none of the mutants did.

FIG. 16A-D. Effectors from non-haustorial fungal pathogens enter via RXLR-mediated PI-3-P binding. A. N-terminus of AvrLm6 (SEQ ID NO: 319) showing functional and non-functional RXLR motifs. 1 mg/mL GFP fusion proteins were incubated with soybean root cells for 12 hr then washed for 2 hr. B. N-terminus of Avr2 (SEQ ID NO: 326) showing functional and non-functional RXLR motifs. 1 mg/mL GFP fusion proteins were incubated with soybean root cells for 12 hr then washed for 2 hr. C. Binding of AvrLm6-GFP to lipids in filter-(left) and liposome-(right) binding assays. D. Binding of Avr2-GFP to lipids in filter-(left) and liposome-(right) binding assays. In both C and D lipids are: PI phosphatidyl (ptd)-inositol; C1P=ceramide-1 phosphate; LPA=lysophosphatidic acid; PA=phosphatidic acid; PS=ptd-serine; PE=ptd-ethanolamine; PC=ptd-choline. In the liposome assays (right panels), B=bound; F=free; M=markers.

FIGS. 17A and B. Binding of three fungal effectors to phosphoinositides. A. N-terminal sequences of effector-like proteins Af2 from Aspergillus fumigatus (Af2; XP 752996.1), CNg2 from Cryptococcus neoformans (Cng2; AAW43853.1) and AvrLm4/7 from Leptosphaeria macularis. Candidate RXLR-like motifs are boxed. B. Sequences shown in A were fused to GFP and the expressed proteins tested for lipid binding using filter-binding assays as described in FIG. 16D.

FIG. 18A-D. PI-3-P is located on the surface of root cells and epithelial cells, but not erythrocytes A. Binding of biosensors to phosphoinositides in filter assays, as described for FIG. 16. B. Binding of biosensors to root cells. Fusion proteins were incubated with soybean root cells for 12 hr then washed for 2 hr. Pairs of fluorescence and light micrographs are shown. Bars=50 μM or 100 μM. C. Binding of biosensors to epithelial cells. Fusion proteins were incubated with cells for 2 hr then washed for 30 min. Pairs of fluorescence micrographs and fluorescence/light overlays are shown. Bars=10 μM. D. Binding of biosensors to human erythrocytes. Fusion proteins were incubated with cells for 2 hr then washed for 30 min. Two independent fluorescence/light overlays are shown. Bars=20 μM.

FIG. 19. Mutations of the Avr1b peptide (SEQ ID NO: 305) containing the RXLR motif assayed using the double barrel particle bombardment assay. Cell entry activity measured as cell death in the presence of Rps1b relative to wild-type Avr1b. Dashes indicate identical residues; indicates Avr1b sequences.

FIG. 20A-C. PI-3-P binding proteins and inositol diphosphate block effector entry A. Blocking binding of effector-GFP fusions into root cells. Fusion proteins were incubated with soybean root cells for 12 hr then washed for 2 hr. Inositol 1,4 diphosphate (500 μM) was preincubated with the fusion proteins for 30 min. 5 mg/ml VAMp7 PX proteins was preincubated with the roots for 2 hr. Pairs of fluorescence and light micrographs are shown. Bars=100 μM. B. Binding of biosensors to epithelial cells. Fusion proteins were incubated with cells for 2 hr then washed for 30 min. Inositol 1,4 diphosphate (500 μM) was preincubated with the fusion proteins for 30 min. 5 mg/ml VAMp7 PX proteins was preincubated with the cells for 2 hr. Overlays of fluorescence and light micrographs are shown. Bars=10 μM. C. Full-length Avr1k protein, with or without the RXLR mutation, was produced in E. coli. 0.25 mg/mL protein was infiltrated into the primary unifoliate leaves of 13 day old seedlings of cultivars Williams (no rps gene) or Williams 82 (Rps1k). Where indicated, the protein was co-infiltrated with 500 χM 1,3IP2. The plants leaves were photographed 5 days after infiltration.

### DETAILED DESCRIPTION

Since many resistance genes against oomycetes encode intracellular proteins, and since several cognate oomycete avirulence genes encode secreted proteins, it has been inferred that there must be a mechanism for translocating the avirulence proteins into the plant cells. Since the RxLR and dEER motifs were first identified during the Phytophthora genome sequence annotation, there has been extensive speculation that these motifs are involved in transporting avirulence proteins into host cells. Importantly, proteins in this family use the N-terminal motifs RxLR and dEER to cross the host plasma cell membrane autonomously, i.e. no other proteins are necessary to effect this translocation. Once inside the host cell, the proteins suppress host defense signaling. The importance of this effector family is underlined by the fact that plants have evolved intracellular defense receptors to detect the effectors and trigger a rapid counter-attack.

The present invention establishes that effectors of fungal plant pathogens contain virulence motifs with consensus sequence BXZ, where B is R, K or H; X is any amino acid and may be absent; and Z is a hydrophobic amino acid, generally L, M, I, W, Y or F. The sequence BXZ represents three contiguous amino acids, or two contiguous amino acids is X is absent. The BXZ family of motifs includes exemplary motifs such as RxLR (which may function with a dEER motif, an exemplary RxLR motif being RSLR) and related functional variants thereof (e.g. RRFLR, RRFLR), as well as exemplary motifs RFYR, RYWT, RIYER, etc). The virulence motifs are responsible for binding of the pathogen effector proteins to polar lipids (e.g. phosphatidyl-inositol-3-phosphate (PI-3-P) and/or phosphatidyl-inositol-4-phosphate (PI-4-P) and/or phosphatidic acid) at or near the surface of a host cell. Stimulation of host cell entry by, for example, PI-4-P, and inhibition by inositol 1, 4 diphosphate suggests that the binding of effectors to polar lipids (such as phosphoinositides, phospholipids and sphingolipids) mediates cell entry of the effectors. All effectors that were tested could also enter human cells, suggesting that this mode of effector entry may be very widespread in plant and animal (including human) pathogenesis, including that which utilizes the Plasmodium Pexel (“P”) motif. Identification of this broad spectrum of effectors containing virulence motifs thereof constitutes a novel target class, and raises the possibility of targeted blockade of pathogen effector proteins. This knowledge can be exploited to develop new classes of antibiotic treatments to prevent a wide variety of pathogenic fungal, Plasmodial and oomycete infections in plants and animals.

The invention also identifies the sequence requirements for the function of the virulence motifs and/or domains. With respect to the BXZ motif, it has been determined that generally only the presence of arginine, lysine or histidine at the first position and the presence of leucine, isoleucine, methionine, tyrosine, phenylalanine or tryptophan at the third position are required to enable function. However, in some embodiments, methionine or leucine at the second position may allow function if none of leucine, isoleucine, methionine, tyrosine, phenylalanine or tryptophan are present at the third position. In some embodiments, the sequences flanking the motifs are required for function. As used herein the term “domain”, in some embodiments, refers to a region or regions of the primary sequence of an effector protein containing more than one virulence motif, e.g. both the RxLR and dEER motifs, or one or more analogous virulence motifs as described herein. The sequence requirements can be defined by a hidden markov model. For example, mutational analysis of the RxLR motif shows that, in some embodiments, the requirement for the first and third positions are quite strict. Furthermore, reversing the order of residues 1 and 2 or of 3 and 4 also abolishes activity, indicating that the mere presence of positive charge and hydrophobicity within the motif are insufficient. The arginine at position 4 is more flexible and can be replaced by lysine or glutamine. Naturally occurring functional variants of RxLR include lysine, histidine, threonine, glycine and alanine at the fourth position.

As a result of these findings, the invention provides methods to inhibit the entry, into a host cell, of effector proteins expressed by pathogens and containing the virulence motifs. The method is carried out by blocking the interaction, for example, by the binding of the motifs to a natural ligand such as a polar lipid, exemplified by phospholipids (e.g. phosphoinositides) and/or sphigolipids. Blocking may be accomplished by any of several means, for example, by exposing one or more virulence motifs to one or more molecules or molecular species which are capable of binding to or otherwise interacting with the virulence motifs, thus preventing the polar lipid (e.g. phosphoinositide, phospholipid or sphingolipid) from binding to the virulence motif. Herein, molecular species such as phosphoinositides, phospholipids and/or sphigolipids which, in nature, bind to one or more motifs of an effector molecule as described herein, causing the effector protein to translocate into the targeted host cell, may be referred to as “natural molecules” or “natural ligands” of the motif. Conversely, the motifs disclosed herein may be considered “natural ligands” of the polar lipids to which they bind. These designations distinguish them from the blocking molecules of the invention, which are added exogenously to cells and used to prevent binding of the natural ligands to the motifs, thereby preventing translocation of the effector protein into the cell. The blocking molecules may or may not be molecules that occur in nature, but if they are, then when used in the present invention, they are isolated or substantially purified, or chemically synthesized.

Blocking molecules of choice include but are not limited to lipid-derived molecules which bind to the motif but not in a manner that results in entry of the effector protein into the cell, e.g. molecules that are sterically related to natural ligands but which do not comprise all requisite properties for enabling translocation of the effector. In other embodiments, the blocking molecules are inositol or inositol derivatives (e.g. various phosphorylated inositols such as inositol monophosphate, various inositol diphosphates such as inositol 1, 4 diphosphate, and other similar molecules); or peptides that bind to the motif and block access to the motif by natural ligands; or peptides that bind to the motif and target the effector for protease degradation; or peptides that bind to the motif and anchor the effector to an external structure such as a cell wall or cell matrix such that the effector cannot enter the cell; or molecules that bind to the motif and cause chemical modification of the effector so that it can no longer enter cells; or other “small molecule” compounds that possess the geometric and charge requisites for binding to one or more of the motifs, thereby blocking the binding of the natural ligand that is responsible for effector translocation.

In one embodiment of the invention, the blocking molecule is a peptide, in particular a peptide with an amino acid primary sequence that is designed to include amino acid residues with charges suitable for interacting with and/or binding to the charged residues of the motif. In such a peptide, the amino acid sequence is designed so that charged atoms or groups (especially of the side chains) are spatially arranged in a manner that allows, for example, negatively charged side chains to be within bonding distance of positively charged side chains of e.g. R residues of the motif, or for aliphatic side chains of the peptide to interact with aliphatic side chains of the motif, etc. Approaches to synthetic peptide design are described, for example, by Devlin et al. (Devlin, H., Panganiban, L. C. and Devlin, P. E. (1990) Random peptide libraries: a source of specific protein binding molecules. Science, 249, 404-406) and Scott and Smith (Scott, J. K. and Smith, G. P. (1990) Searching for peptide ligands with an epitope library. Science, 249, 386-390). Such peptides may be designed to be stable by e.g. by avoiding the use of known protease cleavage sites in the sequence; by introducing various non-natural amino acids; or by various modifications to amino acids (e.g. amidation, sulfonation, etc.) that increase the stability of the molecule, so long a such modifications do not interfere with binding to the effector motif.

The binding or interaction of the blocking molecule(s) may be of any suitable type, and will depend on the nature of the blocking molecule. For example, the binding may be covalent and hence essentially irreversible. Thus, in some embodiments of the invention, the blocking molecule is one that, upon contact with one or more chemically reactive functional groups of the motif or polar lipid, forms a covalent bond with the one or more functional groups that participate in binding, or with functional groups of adjacent portions of the molecule (e.g. adjacent residues of and effector protein) in a manner that blocks access to the motif (e.g. by phospholipids and/or sphingolipids that are natural ligands of the motif) and/or to the polar lipid, which would otherwise permit translocation of the effector into the host cell that the pathogen is trying to infect. Usually, however, the binding is non-covalent and comprises, for example, electrostatic and/or charge interactions, hydrophobic interactions, van der Waals interactions, etc. For a blocking molecule to be effective, a Kd better than ten-fold less than the concentration of the competing natural ligand (polar lipid or motif) in the region of the host membrane is preferred (a lower Kd indicates tighter binding). In this manner, binding of the natural ligand is prevented or at least attenuated or slowed so as to render the natural ligands ineffective in enabling the effector molecule to enter the targeted host cell, and infection of the host by the pathogen which manufactured the effector molecule is prevented, or attenuated or slowed. Those of skill in the art will recognize that much benefit can accrue from a treatment that inhibits a pathogen, even if inhibition is not absolute, but merely attenuates or slows the symptoms of infection. “Inhibition” or “prevention of infection” as used herein is intended to encompass all such degrees of inhibition, blocking, etc. In addition, in the case of plants, the overall biomass of harvested plants or plant products, and therefore the usefulness of the crop, may be advantageously increased even if some plants remain affected by the pathogen after treatment as described herein.

“Motifs” to which natural ligand binding is blocked include BXZ motifs as described herein, as well as RxLR motifs, dEER motifs, the Pexel motif, and the RYWT, RIYER, RSLR, RRLLR, RRFLR, and RFYR motifs. Those of skill in the art will recognize that many effector proteins contain both an RxLR motif and a dEER motif, or one or more virulence motifs as described herein. According to the invention, in one embodiment, the binding of a blocking molecule (which in some embodiments may be a natural ligand such as a phospholipid or sphingolipid) to one or more (e.g. either one or the other, or both, of the RxLR and dEER motifs) is blocked, and blocking occurs in a manner that prevents the effector protein that bears the motif(s) from entering the host cell. In another embodiment, the binding of a natural ligand to a Pexel motif is blocked, and blocking occurs in a manner that prevents the effector protein that bears the motif from entering a host cell. In another embodiment, the binding of a natural ligand to a RYWT, RIYER, RSLR, RRLLR, RRFLR, and/or RFYR motif is blocked, and blocking occurs in a manner that prevents the effector protein that bears the motif from entering a host cell. In some embodiments, the blocking molecule binds to or interacts directly with residues of one or more of the virulence motifs, if two or more virulence motifs are present in an effector. However, this need not always be the case, as binding to a single motif may be sufficient. In other embodiments, the blocking molecule (or molecules) binds to or interacts with adjacent residues. “Adjacent residues” may, but need not necessarily be, adjacent in primary sequence to the motif. They may also be in proximity due to the secondary or tertiary structure of the effector molecule. For example, in one embodiment, the effector protein comprises an RXLR motif followed by at least one aspartate or one glutamate residue within a 60 amino acid carboxy terminal flanking sequence. In other words, within the effector protein, the sequence which is attached directly to the carboxy terminus of the RxLR motif (which, in primary sequence, follows immediately after the carboxyl terminal R of the motif) contains at least one aspartate residue and/or at least one glutamate residue within the first 60 amino acids of the sequence.

In addition, the RxLR motifs that are targeted for blocking by the methods of the invention include but are not limited to those which comprise at least one of a two or three amino acid sequence selected from the group consisting of: arginine, any amino acid, leucine; histidine, any amino acid, leucine; lysine, any amino acid, leucine; arginine, any amino acid, isoleucine; histidine, any amino acid, isoleucine; lysine, any amino acid, isoleucine; arginine, any amino acid, methionine; histidine, any amino acid, methionine; lysine, any amino acid, methionine; arginine, any amino acid, tyrosine; histidine, any amino acid, tyrosine; lysine, any amino acid, tyrosine; arginine, any amino acid, phenylalanine; histidine, any amino acid, phenylalanine; lysine, any amino acid, phenylalanine; arginine, any amino acid, tryptophan; histidine, any amino acid, tryptophan; lysine, any amino acid, tryptophan; arginine, any amino acid, valine; histidine, any amino acid, valine; lysine, any amino acid, valine; arginine, leucine; histidine, leucine; lysine, leucine; arginine, isoleucine; histidine, isoleucine; lysine, isoleucine; arginine, methionine; histidine, methionine; lysine, methionine; arginine, tyrosine; histidine, tyrosine; lysine, tyrosine; arginine, phenylalanine; histidine, phenylalanine; lysine, phenylalanine; arginine, tryptophan; histidine, tryptophan; lysine, tryptophan; arginine, valine; histidine, valine; and lysine, valine. In some embodiments, the R of the RxLR motif is preceded by R, i.e. the motif is RRxLR, with “x” being any amino acid, or in particular L or F. These particular sequence may be represented by standard conventions using the single letter abbreviation for the amino acid and an “x” for the variable residue, e.g. as RXL for “arginine, any amino acid, leucine”.

In addition, blocking may be accomplished by exposing one or more of the polar lipids to which the motifs bind (i.e. “target lipids”) to one or more molecules or molecular species which are capable of binding to or otherwise interacting with the targeted polar lipid, thus preventing the motif (and hence the effector molecule) from binding to the polar lipid. In this embodiment, blocking molecules include but are not limited to: peptides, proteins, and other molecules which bind the polar lipid(s), for example, peptide mimetics of one or more effector motifs, small molecules or drugs which bind the polar lipids, various charged species which bind to the polar lipids, etc. The blocking molecule may bind to the natural target lipid (e.g. phosphoinositide, phospholipid, sphingolipid or other polar lipid) in order to block the binding of the effector to its target. In one embodiment the blocking molecule may be a naturally occurring protein that binds to the target lipid, such as a protein containing, for example, a C1, C2, PH, FYVE, PX, ENTH, ANTH, BAR, FERM, PDZ, and tubby domains (Stahelin, R. V. (2009). Lipid binding domains: more than simple lipid effectors. Lipid Res 50 Suppl, S299-304). In another embodiment, the blocking molecule may be a peptide with an amino acid primary sequence that is designed to include amino acid residues with charges suitable for interacting with and/or binding to the target phosphoinositide, phospholipid or sphingolipid. In yet another embodiment, the blocking molecule may be a polypeptide (e.g. a peptide, polypeptide, etc.) which includes one or more motif sequences and/or is a mimetic of one or more motif sequences. Blocking molecules that target the polar lipid ligands of the motif may bind to any portion of the lipid that prevents effector binding, or even to adjacent molecules or cellular components that sterically interfere with motif-lipid binding.

The host cells that are protected from effector protein invasion include many species of plant and animal cells, including human cells. Examples of plant cells that can benefit from the practice of the invention include but are not limited to: wheat, maize, rice, sorghum, barley, oats, millet, soybean, common bean (e.g. Phaseolus species), green pea (Pisum species), cowpea, chickpea, alfalfa, clover, tomato, potato, tobacco, pepper, egg plant, grape, strawberry, raspberry, cranberry, blueberry, blackberry, hops, walnut, apple, peach, plum, pistachio, apricot, almond, pear, avocado, cacao, coffee, tea, pineapple, passionfruit, coconut, date and oil palm, citrus, safflower, carrot, sesame, common bean, banana, citrus (e.g. orange, lemon, grapefruit), papaya, macadamia, guava, pomegranate, pecan, Brassica species (canola, cabbage, cauliflower, mustard etc), cucurbits (pumpkin, cantaloupe, squash, zucchini, melons etc), cotton, sugar cane, sugar beets, sunflower, lettuce, onion, garlic, ornamental cut flowers, grasses used in lawns, athletic fields, golf courses and pastures (e.g. Festuca, Lolium, Zoysia, Agrostis, Cynodon, Dactylis, Phleum, Phalaris, Poa, Bromua and Agropyron species), etc.

Examples of animal cells that may benefit from the practice of the invention include but are not limited to: humans, cattle, sheep, pigs, goats, horses, cats, dogs, chickens, turkeys, bees, salmon, trout, bass, catfish, shellfish, crayfish, lobsters, shrimp, crabs, etc.

Many types of invasive pathogens may be targeted and their effector proteins prevented from entering host cells by the methods of the invention. Examples of such pathogens include but are not limited to: any Phytophthora species, e.g. Phytophthora infestans, Phytophthora sojae, Phytophthora ramorum, Phytophthora parasitica, Phytophthora capsici, Phytophthora nicotianae, Phytophthora cinnamomi, Phytophthora cryptogea, Phytophthora drechsleri, Phytophthora cactorum, Phytophthora cambivora, Phytophthora citrophthera, Phytophthora citricola, Phytophthora megasperma, Phytophthora palmivora, Phytophthora Megakarya, Phytophthora boehmeriae, Phytophthora kernoviae, Phytophthora erythroseptica, Phytophthora fragariae, Phytophthora heveae, Phytophthora lateralis, Phytophthora syringae; any Pythium species, e.g. Pythium ultimum, Pythium aphanidermatum, Pythium irregulare, Pythium graminicola, Pythium arrhenomanes, Pythium insidiosum; any downy mildew species; any Peronospora species, e.g. Peronospora tabacina, Peronospora destructor, Peronospora sparsa, Peronospora vicaie; any Bremia species, e.g. Bremia lactucae; any Plasmopora species, e.g. Plasmopora viticola, Plasmopara halstedii; any Pseudoperonospora species, e.g. Pseudoperonospora cuhensis, Pseudoperonospora humuli; any Sclerospora species e.g. Sclerospora graminicola; any Peronosclerospora species, e.g. Peronosclerospora philippinesis, Peronosclerospora sorghi, Peronosclerospora sacchari; any Sclerophthora species, e.g. Sclerophthora rayssiae, Sclerophthora macrospora; any Albugo species, e.g. Albugo candida; any Aphanomyces species, e.g. Aphanomyces cochlioides, Aphanomyces euteiches, Aphanomyces invadans; any Saprolegnia species, e.g. Saprolegnia parasitica; any Achlya species; any rust fungi; any smut fungi; any bunt fungi; any powdery mildew fungi; any Puccinia species, Puccinia striiformis, Puccinia graminis, Puccinia triticina (syn. Puccinia recondita), Puccinia sorghi, Puccinia schedonnardii, Puccinia cacabata; any Phakopsora species, e.g. Phakopsora pachyrhizi, Phakopsora gossypii; any Phoma species, e.g. Phoma glycinicola; any Ascochyta species, e.g. Ascochyta gossypii; any Cryphonectria species, e.g. Cryphonectria parasitica; any Magnaporthe species, e.g. Magnaporthe oryzae; any Gaeumannomyces species, e.g. Gaeumannomyces graminis; any Synchytrium species, e.g. Synchytrium endobioticum; any Ustilago species, e.g. Ustilago maydis, Ustilago tritici, Ustilaginoidea virens; any Tilletia species, e.g. Tilletia indica, Tilletia caries, Tilletia foetida, Tilletia barclayana; any Erysiphe species, e.g. Erysiphe necator (formerly Uncinula necator); any Blumeria species, e.g. Blumeria graminis; Podosphaera oxyacanthae; any Alternaria species, e.g. Alternaria alternata; any Botrytis species, e.g. Botrytis cinerea; any Diaporthe species, e.g. Diaporthe phaseolorum; any Fusarium species, e.g. Fusarium graminearum, Fusarium oxysporum (e.g. f.sp. lycopersici), Fusarium moniliforme, Fusarium solani; any Leptosphaeria species, e.g. Leptosphaeria maculans, Leptosphaeria maydis; any Macrophomina species, e.g. Macrophomina phaseolina; any Monilinia species, e.g. Monilinia fructicola; any Mycosphaerella species, e.g. Mycosphaerella graminicola, Mycosphaerella fijiensis, Mycosphaerella tassiana, Mycosphaerella zeae-maydis; any Phialophora species, e.g. Phialophora gregata; any Phymatotrichopsis species, e.g. Phymatotrichopsis omnivora; any Taphrina species, e.g. Taphrina deformans; any Aspergillus species, e.g. Aspergillus flavus, Aspergillus parasiticus, Aspergillus famigatus; any Verticillium species, e.g. Verticillium dahliae, Verticillium albo-atrum, Rhizoctonia solani, Ophiostoma ulmi (syn. Ceratocystis ulmi), Ophiostoma novo-ulmi; any Septoria species, e.g. Septoria avenae; any Pyrenophora species, e.g. Pyrenophora tritici-repentis; any Colletotrichum species, e.g. Colletotrichum graminicola; any Sclerotinia species, e.g. Sclerotinia sclerotiorum; any Sclerotium species, e.g Sclerotium rolfsii; any Thielaviopsis species, e.g Thielaviopsis basicola; any Coccidioides species, e.g. Coccidioides immitus; any Paracoccidioides species, e.g. Paracoccidioides braziliensis; any Pneumocystis species, e.g. Pneumocystis carinii; any Histoplasma species, e.g. Histoplasma capsulatum; any Cryptococcus species, e.g. Cryptococcus neoformans; any Candida species, e.g. Candida albicans; any apicomplexan parasite species such as: any Plasmodium species, e.g. Plasmodium falciparum, Plasmodium vivax, Plasmodium ovale, Plasmodium malariae; any Babesia species, e.g. Babesia bovis, Babesia bigemina; any Cryptosporidium species, e.g. Cryptosporidium parvum; any Toxoplasma species, e.g. Toxoplasma gondii; any Trypanosomatid species such as: any Trypanosoma species, e.g. Trypanosoma brucei, Trypanosoma cruzi, Trypanosoma congolense, Trypanosoma vivax; any Leishmania species, e.g. Leismania donovani. Any amebozoan parasites; any Entamoeba species, e.g. Entamoeba histolytica; any Mastigamoeba species; any Schistosoma species; any Onchocerca species; any Giardia species; any microsporidial species; any Enterocytozoon species; any Encephalitozoon species, e.g. Encephalitozoon cuniculi, etc.

As a result of the practice of the methods of the invention, entry of a pathogen effector protein into the host cell is prevented, inhibited, slowed, or otherwise decreased or lessened. As a result, the host cell can mount a robust or normal immune response to the pathogen, and infection of the host organism is averted (prevented), or the degree of infection (i.e. the deleterious symptoms that typically accompany the presence of an infection by the pathogen) are eliminated or decreased. Thus, some aspects of the invention also include methods of preventing or attenuating the symptoms of infection usually caused in a host organism by a pathogen which employs effector proteins comprising one or more virulence motifs as described herein to enter host cells. In some embodiments, the methods are used to prevent infection and/or symptoms of infection. In other embodiments, infection may have already started but the methods of the invention can be used to curtail the spread of the infection to other organisms, or to lessen the symptoms in an organism that is already afflicted. This is the case, in particular, with Plasmodium infections, where the methods of the invention are especially useful in preventing the subsequent rounds of parasite multiplication after initial infection, or with other pathogens that multiply logarithmically. In addition, the invention provides methods of maintaining a host cell's ability to mount an immune response to a pathogen, the pathogen being one that produces effector proteins that comprise one or more of the motifs described herein, and the method involving blocking the effector protein from entering the host cell by preventing the binding of its natural ligand.

The mode of administration of the blocking molecules of the invention will depend on several factors, including the nature of the molecule and the host. Generally, the blocking molecule will be in a composition or formulation suitable for administration. If the host organism is a plant, application is generally in the form of a foliar spray or watering solution of e.g. an aqueous or oil solution that includes the blocking molecule in a concentration sufficient to block effector molecules of pathogens which are likely to attack the plant. For administration to an animal, which may be a human, any suitable composition, many of which are known in the art, may be employed, e.g. various pills, powders, liquids, injectable formulations, etc. Likewise, any suitable means may be used, including but not limited to by injection (e.g. subcutaneous or intramuscular), inhalation, orally, intranasally, by ingestion of a food product containing the protein, etc. In addition, the compositions may include one or more than one blocking molecule. For example, a preparation for application to plants may include molecules that block the effector proteins of one or of several different types of pathogen. In addition, the blocking molecules may be administered to plants in conjunction with other beneficial substances, such as fertilizers, various pesticides, growth factors, etc. The same is true for administration to animals, where on or more than one type of blocking molecule may be administered, and may be administered in conjunction with other beneficial substances such as chemotherapeutic agents that also have activity against the pathogen.

In some embodiments, a combination of blocking agents is utilized, e.g. two or more agents that act on or effect the effector protein, or two or more agents that acts on or effect the lipid, or a mixture of blocking agents, one or more of which acts on the effector protein, and one or more of which acts on the lipid.

In another aspect, the invention provides elucidation of the structural requirements of the virulence motifs (e.g. BXZ motifs, such as RXLR, RYWT, RIYER and related or similarly functioning motifs) in oomycetes, fungi, and other pathogens, and of the sequences which flank the motifs, thereby allowing, for example, the design of molecules to bind the motif. As a result, the invention also provides methods to predict which genes in the genome of a pathogen are likely to encode effector molecules. This is significant because, as demonstrated herein, the mere presence of a sequence conforming to the RXLR and dEER motif in a protein may not be sufficient to insure that the protein is an effector, i.e. that the protein is able to traverse the cell wall and enter the cell upon binding to phospholipids or sphingolipids. Functional virulence motifs (or functional virulence domains) may have additional requirements, particularly in the flanking sequences, as described herein. In one embodiment, the invention describes a non-random distribution of amino acid residues in the regions flanking the RXLR motif, represented by a position-weight matrix. One method of predicting whether or not a gene encodes a true effector protein involves the use of a hidden markov model (HMM) based on the position-weight matrix. This method may also be applied to the analysis of other BXZ motifs.

The development of this aspect of the invention was based in the observation of differences in activity between two putative RXLR motifs, RXLR1 and RXLR2, as described in detail in Example 1 below. The differences suggested that surrounding sequences are also important to the activity of an RXLR motif. To define the differences in the surrounding sequences a hidden markov model (HMM) was created using the 10 amino acid residues to the left and right of the RXLR motifs of all of known P. sojae and P. ramorum Avh genes. Using this HMM, the sequences surrounding RXLR2 (which was an authentic, functional motif) had a high score of 18.5, representing an excellent match to the consensus flanking sequence, and very unlikely to have been found in a random sequence. In contrast, sequences surrounding RXLR1 (a non-functional motif) had a low, non-significant score of 0.0. Using the same HMM, the sequences surrounding the RXLR motif of P. infestans Avr3a scored 10.9. Using a similar HMM derived from Hyaloperonospora parasitica Avh genes, the sequences surrounding the RXLR motifs of the H. parasitica Atr1 and Atr13 proteins had scores of 9.8 and 6.3 respectively. These and other comparisons described in Example 1, showed that HMM scores of zero, such as that of Avr1b RXLR1, are characteristic of RXLR strings found at random, i.e. such sequences are not likely to represent true RXLR motifs. On the other hand, HMM scores over 5.0 are characteristic of non-random occurrences of RXLR strings, and the proteins in which such non-random strings occur are likely to be authentic functional RXLR sequences. In other words, such sequences are likely, upon binding a phospholipid or sphingolipid, to promote or allow the translocation, into a host cell, of effector (avirulence) proteins in which they are located or of which they form a part. HMM scores between 0 and 5 are equivocal and cannot be assigned to either category of protein (random RXLR strings vs authentic RXLR motifs). This methodology may also be applied to other virulence motifs, e.g. RYWT, RIYER, etc.

For further discussions of the use of a hidden markov model, see U.S. Pat. No. 6,128,587 to Sjolander (Oct. 3, 2000), the complete contents of which are hereby incorporated by reference.

The invention also provides a method for screening compounds to identify those that inhibit binding of phosphoinositides, phospholipids or sphingolipids (or other natural lipid ligands) to a BXZ motif (e.g. to an RXLR motif, and optionally also to a dEER motif) in an effector protein, and which thus can inhibit translocation of effector proteins that have these motifs into cells. The method involves exposing a candidate or putative blocking compound to one or more BXZ virulence motifs (e.g. one or both of the RXLR and dEER motifs, and/or one or both of RYWT and RIYER) under conditions suitable for binding of either the natural phospholipid/sphingolipid ligands or phosphoinositides to the motifs (phosphoinositides are known to be capable of such binding). The screening is evaluated in that if the candidate compound is able to bind to one or more of the virulence motifs, and especially to two motifs when they are found to be present in a single effector (e.g. RXLR and dEER) then the compound is selected as a compound that will inhibit binding of the natural ligands to the motifs in an effector protein, and prevent entry of the effector protein into a cell. This is especially the case if the blocking compound is able to competitively bind to the motif in the presence of a natural ligand.

Alternatively, such screening methods may be adapted and used to identify blocking compounds which bind to or otherwise interfere with the polar lipid to which the motif binds.

Blocking of effector-lipid binding may also be prevented or attenuated using genetic engineering/molecular biology techniques. Such techniques may target one or both of the effector (e.g. the motif sequence) and the lipid to which a motif binds. For example, host cells (e.g. in plants) may be genetically engineered to express inhibitory RNA (e.g. siRNA) that inhibits one or more enzymes involved in synthesis of a target lipid, or in transport of the lipid to the cell surface where it is accessible to the motif. Alternatively, host cells may be genetically engineered to produce peptides or proteins which contain one or more motifs, in a manner that promotes expression of the peptides/proteins and their binding to at least one target lipid. Other strategies will occur to those of skill in the art, and all such methods are encompassed by the invention.

The invention also provides methods for preventing pathogens from invading or infecting cells, by prophylactic ally applying one or more of the blocking compounds described herein to a substrate with which a pathogen and a cell the pathogen might infect may come in contact. The method inhibits entry, into the cell, of a pathogenic effector protein (entry of the pathogenic effector protein requiring binding of at least one motif of the effector protein as described herein to at least one polar lipid of the cell) and comprises the step of contacting a substrate which contains or is likely to contain a pathogen comprising the pathogenic effector protein with a blocking compound as described herein. By “contacting” is meant applying, permeating, coating or otherwise placing the blocking compound on the substrate. The blocking compound is capable of i) binding to at least one motif of the pathogenic effector protein; or ii) binding to at least one polar lipid of said cell (the polar lipid being the cellular ligand of the effector protein motif). Binding of the blocking compound prevents entry of the pathogenic effector protein into said cell (and hence prevents infection of the cell by the pathogen), if the pathogen comes into contact with the substrate. The blocking agent may be applied to the substrate by any suitable means, e.g. by spraying, painting, coating, etc., or even by manufacturing the substrate to contain the blocking agent (e.g. fabric), or by permeating or soaking the substrate with the blocking agent, etc. The substrate may be any suitable substrate that may be contacted by the pathogen, and which usually will be or may also come into contact with a cell which might be infected by the pathogen, or in some cases may be the cell or a collection of cells which may encounter or be exposed to the pathogen. Exemplary substrates include but are not limited to: plants (e.g. to leaves, fruit, roots, etc.) for example, by spraying or otherwise placing the blocking agent, sometimes, though not always, on an exterior surface of the plant (e.g. mature plants, plants “in the field”, plants in green houses, seedlings, seeds, sprouts, etc.); fabrics (e.g. fabrics used for tents, mosquito netting, clothing, etc.), water (e.g. bodies of water, swamps, pools of standing water, wells, drinking water, etc.); skin, hair and/or fur, eyes, ear and nasal passages, the mouth, etc., e.g. of an animal (e.g. mammals such as humans, or other mammals, and also reptiles, fish, birds, etc. i.e. veterinary applications are also contemplated). For animals, application may be external by the application of e.g. lotions, sprays, rinses, mists, drops, washes, (e.g. mouthwash), etc; (internal administration is also contemplated, as described above). The substrate may also be an insect, e.g. an insect that is known or suspected of carrying the pathogen which comprises the effector protein, or is capable of synthesizing the effector protein.

The invention is further illustrated by the following Examples, which should not be interpreted as limiting the invention in any way.

### EXAMPLES

### Example 1

### RXLR-mediated entry of Phytophthora sojae effector Avr1b (SEQ ID NO: 2) into soybean cells does not require pathogen encoded machinery

Effector proteins secreted by oomycete and fungal pathogens have been inferred to enter host cells, where they interact with host resistance gene products. Using the effector protein Avr1b of Phytophthora sojae, an oomycete pathogen of soybean, we show that a pair of sequence motifs, RXLR and dEER, plus surrounding sequences, (SEQ ID NO: 46) are both necessary and sufficient to deliver the protein into plant cells. Particle bombardment experiments demonstrate that these motifs function in the absence of the pathogen, indicating that no additional pathogen encoded machinery is required for effector protein entry into host cells. Furthermore, fusion of the Avr1b RXLR and dEER domain to green fluorescent protein (GFP) allows OFF to enter soybean root cells autonomously. The conclusion that RXLR and dEER serve to transduce oomycete effectors into host cells indicates that the more than 370 RXLR and dEER containing proteins encoded in the genome sequence of P. sojae are candidate effectors. We further show that the RXLR and dEER motifs can be replaced by the closely related erythrocyte targeting signals found in effector proteins of Plasmodium, the protozoan that causes malaria in humans. Mutational analysis of the RXLR motif shows that the required residues are very similar in the motifs of Plasmodium and Phytophthora. Thus the machinery of the hosts (soybean and human) targeted by the effectors may be very ancient.

RXLR2 and dEER Motifs of Avr1b are Required for its Avirulence Function in Transgenic P. sojae Lines

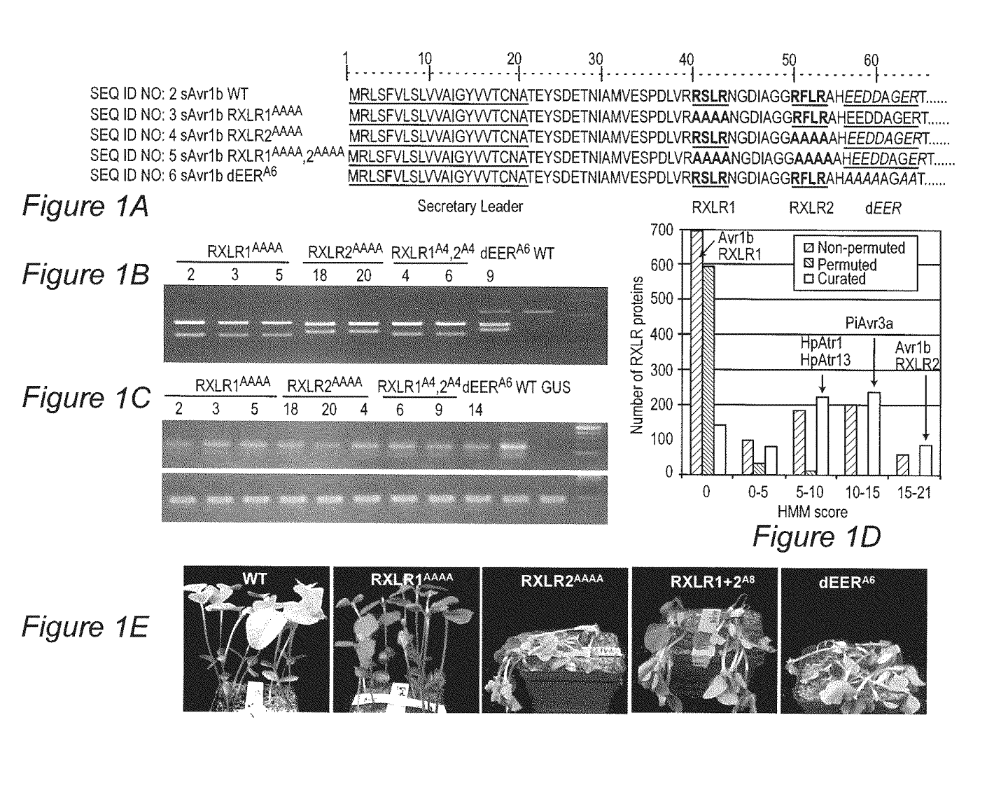

To test the function of the RXLR and dEER motifs of Avr1b, transgenic P. sojae strains that expressed either wild-type or mutant Avr1b-1 genes were created. Wild-type Avr1b contains two RXLR motifs, RXLR1 and RXLR2 (FIG. 1A). Mutations in either or both of the RXLR motifs (SEQ ID NO: 3,4,5), in addition to a mutation in the BEER motif (FIG. 1A) (SEQ ID NO: 6), were created. The Avr1b-1 gene constructs were fused to a strong constitutive promoter, HAM34 (Judelson, H., Tyler, B. M., and Michelmore, R. W. 1991. Mol. Plant-Microbe Interact. 4, 602-607.), and introduced into a strain, P7076, that expresses a variant Avr1b protein that does not confer avirulence against Rps1b-containing soybeans (Shan, W., Cao, M., Leung, D., and Tyler, B. M. 2004. Mol. Plant-Microbe Interact. 17, 394-403). Two independent transformants (T17 and T20) expressing wild type Avr1b-I (FIG. 1B, C) lost the ability to infect soybean plants carrying Rps1b, but were unaffected in their ability to infect plants lacking Rps1b (FIG. 1E, Table 1). This demonstrated that they had acquired avirulence against Rps1b as a result of a functional Avr1b gene product. This result was confirmed using two different pairs of isolines of soybean that differed only in the presence of Rps1b, namely Williams (no Rps gene) with L77-1863 (Rps1b; Williams background) and HARO(1-7)1 (No Rps; Harosoy background) with HARO13 (Rps1b; Harosoy background) (FIG. 1E; Table 1).

In contrast, in five independent transformants expressing the RXLR2^{AAAA} (SEQ ID NO 4) mutant, there was no gain of avirulence against Rps1b cultivars, despite the presence of abundant mRNA from the transgene (FIG. 1E, Table 1). Thus, the RXLR² motif is necessary for Avr1b activity when the protein is delivered by the pathogen. Since the RXLR¹ motif was intact in the RXLR2^{AAAA} mutant, the motif appeared to be non-functional. Consistent with this inference, the RXLR1^{AAAA} mutation (SEQ ID NO 3) did not abolish avirulence in three independent transformants (FIG. 1E, Table 1). As expected, avirulence was lost in the RXLR1^{AAAA}, RXLR2^{AAAA} double mutants (FIG. 1E, Table 1) (SEQ ID NO: 5). A mutation in the dEER motif (SEQ ID NO: 6) also abolished avirulence (in two independent transformants) indicating that this motif is also required for the function of the protein (FIG. 1E, Table 1).

TABLE 1
Molecular characterization and avirulence testing of P. sojae stable
transformants

| | | | | Avirulence | | |
|---|---|---|---|---|---|---|
| | | | | (Surviving | | |
| | Transgene | Expression | Transformant | seedlings)^{d} | | |
| Strains | (PCR)^{a} | (RT-PCR)^{b} | Validation^{c} | Rps1^{b} | rps | p^{e} |

P7076 (Gus)

| | | | | | |
|---|---|---|---|---|---|
| GUS | no | no | 1/23 | 1/22 | 0.77 |

P7076 (sAvr1b WT)

| | | | | | | |
|---|---|---|---|---|---|---|
| T17 | yes | yes | Pst I | 20/30 | 0/21 | 3.9E−07 |
| T20 | yes | yes | Sequence | 25/44 | 2/28 | 1.2E−05 |

P7076 (sAvr1b RXLR1^{AAAA})

| | | | | | | |
|---|---|---|---|---|---|---|
| RXLR1−2 | yes | yes | Pst I | 32/36 | 3/20 | 5.0E−08 |
| RXLR1−3 | yes | yes | Pst I | 31/54 | 3/21 | 6.4E−04 |
| RXLR1−5 | yes | yes | Pst I | 37/57 | 7/23 | 5.1E−03 |

P7076 (sAvr1b RXLR2^{AAAA})

| | | | | | | |
|---|---|---|---|---|---|---|
| RXLR2−18 | yes | yes | Pst I | 6/44 | 3/17 | 0.48 |
| RXLR2−20 | yes | yes | Pst I | 4/46 | 3/19 | 0.33 |

P7076 (sAvr1b RXLR1^{AAAA}; RXLR2^{AAAA})

| | | | | | | |
|---|---|---|---|---|---|---|
| RXLR1+2−4 | yes | yes | Pst | 5/31 | 2/16 | 0.55 |
| RXLR1+2−6 | yes | yes | Pst I | 4/43 | 1/21 | 0.47 |

P7076 (sAvr1b dEER^{A6})

| | | | | | | |
|---|---|---|---|---|---|---|
| dEER−9 | yes | yes | Sequence | 4/59 | 2/23 | 0.79 |
| dEER−14 | yes | yes | Pst I | 4/40 | 3/15 | 0.28 |

Ps Avr4/6−Avr1bCt

| | | | | | | |
|---|---|---|---|---|---|---|
| 4/6−1b−3 | yes | yes | size | 15/24 | 3/11 | 0.057 |
| 4/6−1b−19 | yes | yes | size | 14/26 | 3/16 | 0.025 |

Hp 341−Avr1bCt

| | | | | | | |
|---|---|---|---|---|---|---|
| 341−1b−13 | yes | yes | size | 16/19 | 0/11 | 6.6E−06 |
| 341−1b−17 | yes | yes | size | 23/24 | 0/7 | 3.0E−06 |

mAvr1bCt

| | | | | | | |
|---|---|---|---|---|---|---|
| mAvr1bCt−4 | yes | yes | size | 3/20 | 3/16 | 0.77 |
| mAvr1bCt−5 | yes | yes | size | 2/21 | 1/15 | 0.63 |

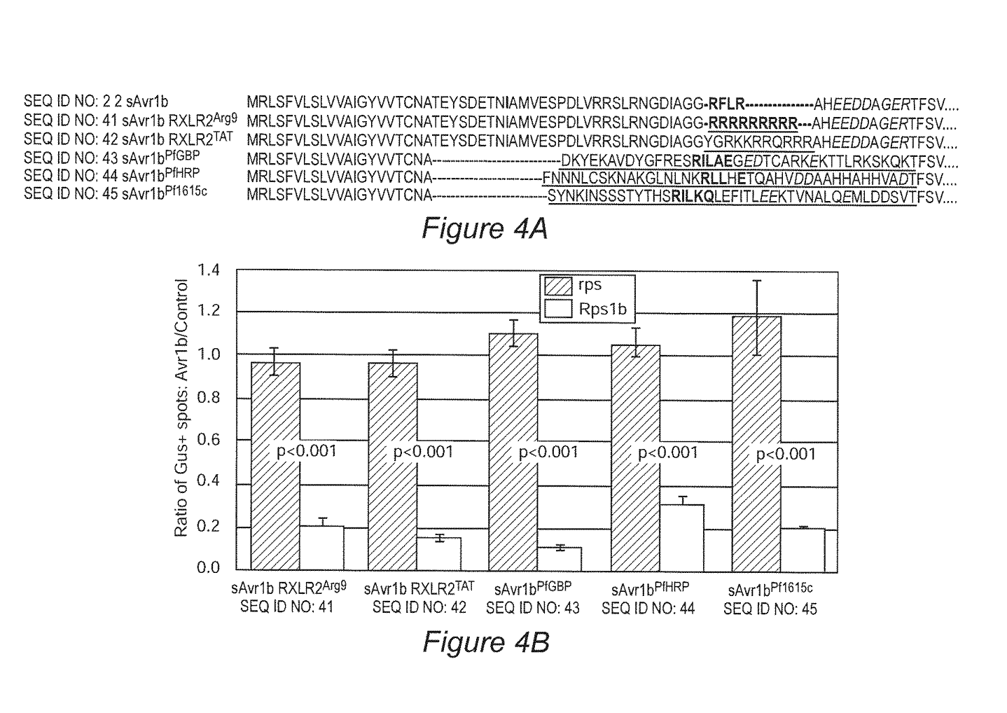

^{a}The presence of transgenes was verifiedby PCR as described in the Materials and Methods. + = transgene present; − = transgene not detected.
^{b}Transgene expression was determined by qualitative RT-PCR (RT-PCR) and by quantitative RT-PCR (q-PCR) as described in the Materials and Methods. Yes = transgene transcripts present; no = transgene transcripts not detected;
ND = not determined.
^{c}The presence of the relevant mutation in the transforming plasmid was verified by sequencing in every case. The presence of the correct mutation within the transgenes of each transformed strain was verified after PCR amplification of the Avr1b−1 transgene by Pst I digestion or by sequencing in the case of the mutants (e.g. FIG. 1) or by size in the case of the Avh gene fusions and N-terminal deletion (e.g. FIG. 4).
^{d}The avirulence of each transgenic strain was tested by inoculation of seedlings containing Rps1b (L77-1863) or no rps gene (Williams), as described in the Materials and Methods. The number of surviving seedlings/total inoculated seedlings is shown, summed from all replicates
^{e}Fisher's exact test (one tailed) was used to compare the frequency of seedling survival between rps and Rps1b plants. A significant p value (0.05) indicates that the transformant's phenotype is avirulent.

The difference in activity between RXLR1 (SEQ ID NO: 3) and RXLR2 (SEQ ID NO: 4) suggests that surrounding sequences are important to the activity of an RXLR motif. To define the differences in the surrounding sequences a hidden markov model (HMM) was created using the 10 amino acid residues to the left and right of the RXLR motifs of all of the P. sojae and P. ramorum Avh genes (Tyler et al., 2006; Jiang et al., 2008). Using this HMM, the sequences surrounding RXLR2 (SEQ ID NO: 5) had a high score of 18.5, representing an excellent match to the consensus flanking sequence, very unlikely to have been found in a random sequence. In contrast, sequences surrounding RXLR1 (SEQ ID NO: 3) had a low, non-significant score of 0.0. Using the same HMM, the sequences surrounding the RXLR motif of P. infestans Avr3a scored 10.9. Using a similar HMM derived from Hyaloperonospora parasitica Avh genes, the sequences surrounding the RXLR motifs of the H. parasitica Atr1 and Atr13 proteins had scores of 9.8 and 6.3 respectively.

To establish the significance of these HMM scores, the Phytophthora HMM was used to score the RXLR motifs of 1240 RXLR-containing sequences identified from a pool of all putative secreted P. sojae and P. ramorum proteins by Jiang et al. (2008). As a control, 639 RXLR-containing sequences were scored found after permuting the sequences of all the putative secreted P. sojae and P. ramorum proteins (Jiang et al., 2008). As shown in FIG. 1D, the RXLR strings of 698 (56%) of the 1240 real proteins had an HMM score of zero, while the RXLR strings of 595 (93%) of the permuted proteins had a zero score, and only 13 (1.8%) scored above 5.0. In contrast, of 765 proteins that Jiang et al. (2008) identified as high quality candidate effectors, only 18% had an HMM score of zero, and 543 (72%) had a score over 5.0. From this comparison we conclude that HMM scores of zero, such as that of Avr1b RXLR1 (SEQ ID NO: 3), are characteristic of RXLR strings found at random, while scores over 5.0 are characteristic of non-random occurrences of RXLR strings and of the RXLR strings of functional avirulence proteins. HMM scores between 0 and 5 are equivocal. The curated Avh genes with a score of zero may represent pseudogenes as many of them were identified principally by C-terminal sequence similarity.

The Interaction Between Avr1b and the Rps1b Gene Product Occurs Within Host Cells and Does Not Require the RXLR and dEER Motifs

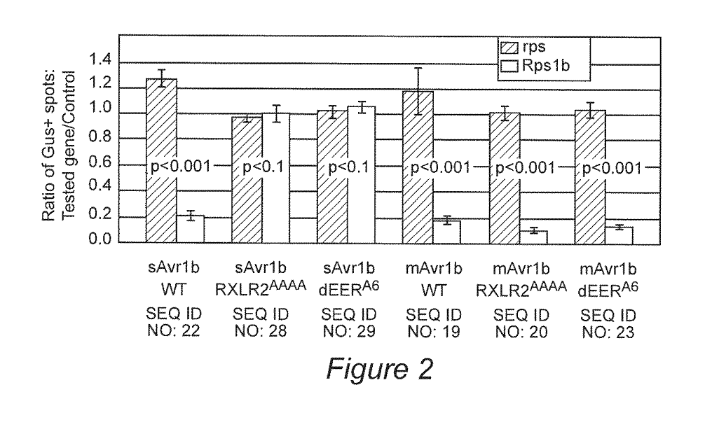

To confirm that the site of interaction of Avr1b with the Rps1b gene product is within the plant cell, particle bombardment was used to introduce DNA encoding Avr1b proteins lacking a secretory leader into soybean cells together with DNA encoding β-glucuronidase (GUS). This assay measures the functional interaction of the Avr1b protein with the intracellular product of the soybean Rps1b gene; when the two proteins interact, programmed cell death is triggered in the transformed cells ablating the development of tissue patches expressing GUS. Since the Avr1b protein lacks its normal secretory leader, the protein should be synthesized in the plant cytoplasm. To facilitate the comparison of test and control bombardments, a novel double-barreled attachment for the Bio-Rad Gene Gun was utilized (Dou et al., 2008). The gun shooting two different DNA samples side-by-side into a leaf in the same shot, which greatly improves the reproducibility of the results (Dou et al., 2008). FIG. 2 shows that delivery of DNA encoding leader-less Avr1b protein (SEQ ID NO: 19) into soybean cells significantly reduced the number of blue GUS-positive patches when the Rps1b gene was present, but not when Rps1b was absent (FIG. 2A). This is consistent with a cytoplasmic location for the Avr1b-Rps1b interaction. When RXLR2 or dEER motifs were replaced by four or six alanine residues, respectively [FIG. 2A, mAvr1b(RXLR2^{AAAA})(SEQ ID NO: 20) and mAvr1b(dEER^{A6}) (SEQ ID NO: 23)], the interaction of the cytoplasmic, leader-less Avr1b with Rps1b was unaffected (FIG. 2A), indicating that the RXLR2 and dEER motifs were not required for the interaction.

RXLR-Mediated Transit into Soybean Cells Does Not Require the Pathogen

To test whether RXLR function requires the presence of the pathogen, the bombardment assay was used to determine the effect of the RXLR2^{AAAA} mutation on secreted Avr1b protein (SEQ ID NO: 18). When soybean cells were bombarded with DNA encoding wild type Avr1b (SEQ ID NO: 22), including its normal secretory leader, a reduction in GUS-positive blue spots was observed comparable to that observed for the non-secreted protein [FIG. 2, sAvr1b(WT)]. However when the RXLR2^{AAAA} (SEQ ID NO: 28) or dEERA6 (SEQ ID NO: 29) mutations were present in the bombarded DNA, there was no reduction in the number of blue spots [FIG. 2, sAvr1b(RXLR2^{AAAA}) (SEQ ID NO: 11) and sAvr1b(dEER^{A6}) (SEQ ID NO: 13)]. From these results it may be inferred first that the secretory leader is functional in soybean and targets Avr1b protein to the outside of the cell; second, that the RXLR2 (but not RXLR1) and dEER motifs are required for Avr1b protein to re-enter the cell, which confirms the conclusion from the P. sojae transformation experiments. Importantly, the results also show that RXLR-dEER-mediated entry does not require the presence of the pathogen.

To support the inference that the secretory leader of Avr1b was correctly exporting the protein from the plant cells in the bombardment assay, a gene encoding Aequorea coerulescens green fluorescent protein (acGFP; “GFP” herein) fused either to the Avr1b leader (SEQ ID NO: 27) or to full-length Avr1b (SEQ ID NO: 25) was constructed. These fusions enabled tracking of the proteins and checking their stability. To aid in visualization onion bulb epidermal cells were used rather than soybean cells. GFP was exported from the cells and accumulated in the apoplast when the secretory leader was attached to GFP but accumulated in the cytoplasm and nucleus when the leader was not attached (not shown). When full length Avr1b was fused to GFP (SEQ ID NO: 25), the proteins also accumulated in the apoplast if a mutation was present in either the RXLR2 motif (SEQ ID No: 28) or in the dEER motif (SEQ ID No: 29). This observation confirmed that the protein encoded by these mutants was stable and correctly targeted outside of the cells. When cells expressing Avr1b-GFP fusion proteins with RXLR mutations were plasmolyzed by treatment with 0.8M mannitol for 15 min, the GFP was associated with the cell wall and not with the plasma cell membrane (not shown). Furthermore, GFP protein could be seen diffusing into the apoplast between pairs of neighboring cells not shown). Similar observations were made when cells expressing secreted GFP or Avr1b-GFP fusion proteins with a dEER mutation were plasmolyzed (not shown). If the RXLR2 and dEER motifs were intact however, the sAvr1b-GFP protein fusion accumulated in the cytoplasm and nucleus of the cells, similar to the mAvr1b-GFP (SEQ ID NO: 24) fusion lacking the leader. When cells expressing sAvr1b-GFP fusion proteins were plasmolyzed by treatment with 0.8M mannitol for 15 min, the GFP could be observed to have either fully or partially returned to the inside of the cells. These results supported our conclusion that the RXLR2 and dEER motifs act together to enable Avr1b protein to re-enter the plant cells.

The Avr1b RXLR and dEER Motifs are Sufficient to Target GFP to Soybean Cells

The RXLR and dEER region of Avr1b was fused to GFP (SEQ ID NO: 46), and the fusion protein was synthesized in E. coli and partially purified. Root tips of soybean seedlings were incubated with the isolated fusion protein for 12 hours, washed for four hours in water, then observed under light and UV microscopy to localize the OFF. GFP accumulated inside many of the root cells, whereas buffer alone did not produce any fluorescence. The optical sections produced by the confocal microscope revealed that the protein penetrated approximately 10 cell layers deep during the 12 hour incubation. The characteristic accumulation of GFP in the nuclei of the treated cells is comparable to the pattern observed when GFP is expressed in planta, and verifies that the GFP is located inside the cells. The nuclear localization of the protein also indicates that the cells are alive. If mutations were present in the RXLR or dEER motifs of the fusion protein, GFP did not accumulate inside the soybean root cells. When the RXLR and dEER region was replaced by the artificial protein transduction motif Arg9 (SEQ ID NO: 112), GFP once again entered the soybean root cells and accumulated in the nuclei.

Avr1b RXLR and dEER Motifs can be Replaced by RXLR and dEER-Containing Protein Sequences Encoded by Bioinformatically Identified Avh Genes

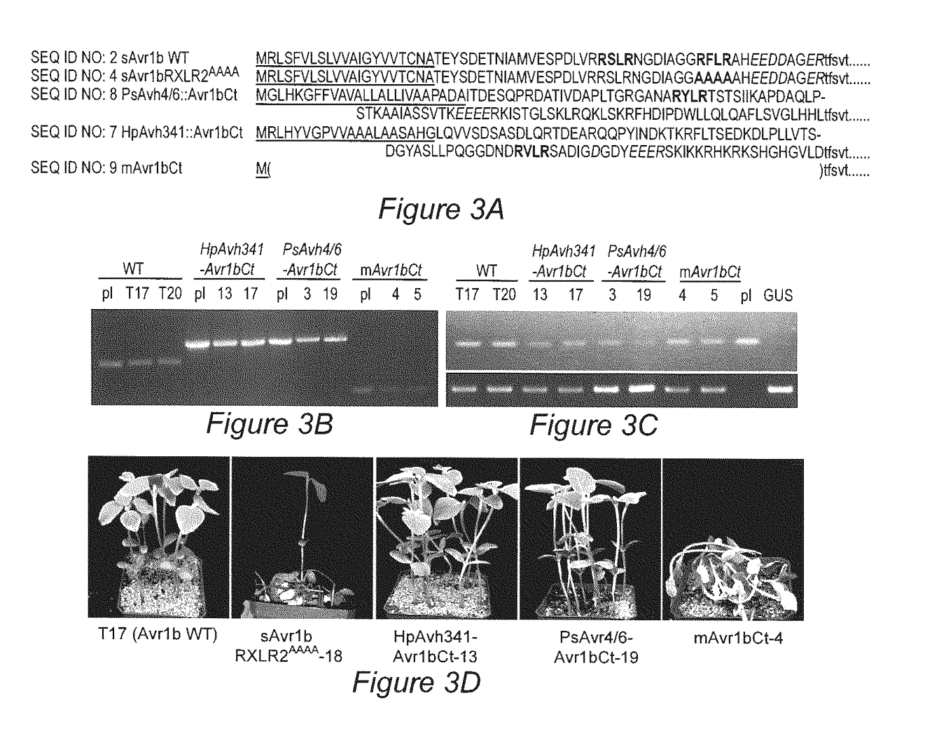

To determine if the RXLR and dEER motifs of bioinformatically identified Avh genes could functionally replace the RXLR2 and dEER motifs of Avr1b-1, full length Avh genes from P. sojae and H. parasitica were fused to an Avr1b-1 N-terminal deletion mutant lacking the RXLR and dEER motifs. The fusion genes were then introduced into P. sojae and the transformants were tested for avirulence on Rps1b-containing soybean cultivars. Both Avh genes, P. sojae Avh171 (since identified as Avr4/6; Dau et al., 2008) (SEQ ID NO: 8) and H. parasitica Avh341 (SEQ ID NO: 7), could replace the requirement for the RXLR2 and dEER motifs as judged by the avirulence of the transformants on Rps1b-containing cultivars, whereas transformants containing only the C-terminus of Avr1b fused to an initiator methionine remained virulent (FIG. 3 and Table 1). This result indicates that the RXLR and dEER motifs form a distinct transferable functional domain of Avr1b and other Avh proteins. The HMM scores of the RXLR-dEER motifs of Ps Avr4/6 and Hp Avh341 are both well within the functional range (6.9 and 14.2, respectively).

### The Avr1b Host Targeting Signal can be Functionally Replaced by Autonomous Protein Transduction Motifs

Protein transduction domains (PTDs) capable of autonomously carrying proteins across plasma cell membranes have been described and characterized in the HIV-1 Tat protein. Arginine-rich peptides such as Arg9 can also carry out this function. To compare RXLR and dEER mediated effector delivery with the function of PTDs, the RXLR2 motif of Avr1b was replaced with the TAT PTD (SEQ ID NO: 42) or with Arg9 (FIG. 4A) (SEQ ID NO: 41). The resultant proteins were treated using the particle bombardment assay, and both PTDs could functionally replace the RXLR2 motif of Avr1b, restoring the avirulence reaction of Avr1b with Rps1b (FIG. 4B). Furthermore, when the version of secreted Avr1b that contained the Arg9 sequence in place of the RXLR2 motif was fused to GFP (SEQ ID NO: 30), the fusion protein accumulated in the cytoplasm and the nucleus of bombarded onion bulb cells rather than the apoplast, confirming that Arg9 could functionally replace RXLR2 (not shown). Similar results were obtained when the version of secreted Avr1b that contained the TAT PTD was fused in place of the RXLR2 motif to GFP (SEQ ID NO: 31). Finally, when Arg9 was fused to GFP (SEQ ID NO: 112), the isolated proteins could enter soybean root cells directly (not shown).

The Avr1b Host Targeting Signal is Interchangeable with Host Targeting Signals from Plasmodium Effectors

To test if the erythrocyte targeting signals of Plasmodium effector proteins could functionally replace the RXLR and dEER region of Avr1b, the residues of Avr1b from the end of the secretory leader to the end of the dEER motif were replaced with the mature N-termini of three different Plasmodium effector proteins that are targeted to the erythrocyte cytoplasm, namely PfGBP-130 (SEQ ID NO: 121), PfHRPII (SEQ ID NO: 123) and PfPFE1615c (SEQ ID NO: 125) (Bhattacharjee, S., Hiller, N. L., Liolios, K., Win, J., Kanneganti, T. D., Young, C., Kamoun, S., and Haldar, K. 2006. PLoS Pathog 2, e50.). The entire 37-41 amino acid region of each Plasmodium effector required for transduction was used (FIG. 4A). As shown in FIG. 4B, all three Plasmodium host targeting domains could functionally replace the Avr1b N-terminus in targeting Avr1b to the soybean cytoplasm, assuming that they do not simply interfere with secretion.

### Functional Characterization of the RXLR Motif

To experimentally characterize the sequence requirements of the RXLR motif, a series of mutations were introduced into the motif in a version of the Avr1b-1 gene that retained the secretory leader, and assayed the mutants using the bombardment assay (Table 2). Mutations which targeted the arginine at position 1 or the leucine at position 3 has the strongest effect on the ability of Avr1b to ablate GUS-positive tissue patches. Replacement of R1 with lysine reduced function significantly (33% ablation compared to 78%; p<0.001) while glutamine replacement completely abolished it. Replacement of L3 with alanine or even the relatively conservative valine also completely abolished function. Replacement of the arginine at position 4 with a glutamine slightly but significantly reduced function (58% ablation compared to 72%; p<0.001). Reversing the order within the first and second two pairs of positively-charged and hydrophobic residues (RFLR->FRLR; RFLR->RFRL) completely abolished avirulence activity, indicating that positions of R1 and L3 were critical, not just their presence.

TABLE 2
Function of RXLR2 mutants of Avr1b assayed
by particle bombardment

| | | Ratio of GUS-positive | | | | |
|---|---|---|---|---|---|---|
| SEQ | RXLR2 | spots^{b} | | abla- | | |
| ID NO | sequence^{a} | rps | Rps1b | tion^{c} | p value^{d} | Activity |
| 22 | RFLR | 1.26 ± 0.07 | 0.28 ± 0.03 | 0.78 | <0.001 | Yes |
| 20 | AAAA | 0.93 ± 0.04 | 0.96 ± 0.05 | 0 | >0.1 | No |
| 38 | KFLR | 1.04 ± 0.04 | 0.70 ± 0.04 | 0.33^{e} | <0.001 | Partial |
| 37 | QFLR | 0.95 ± 0.03 | 0.99 ± 0.03 | 0 | >0.1 | No |
| 31 | FRLR | 1.00 ± 0.04 | 0.98 ± 0.05 | 0 | >0.1 | No |
| 34 | RFLQ | 0.98 ± 0.07 | 0.41 ± 0.08 | 0.58^{e} | <0.001 | Partial |
| 36 | QFLQ | 1.03 ± 0.05 | 1.05 ± 0.05 | 0 | >0.1 | No |
| 35 | RFAR | 0.94 ± 0.03 | 0.91 ± 0.05 | 0 | >0.1 | No |
| 40 | RFVR | 0.95 ± 0.05 | 1.03 ± 0.07 | 0 | >0.1 | No |
| 33 | RFRL | 1.02 ± 0.04 | 0.96 ± 0.04 | 0 | >0.1 | No |

^{a}Amino acid sequence of RXLR2 in wild-type and mutants. RFLR is the wild-type. Altered residues are underlined.
^{b}Ratio of blue spots in the presence of various RXLR2 mutants of Avr1b−1, compared to the control empty vector when bombarded onto leaves from rps plants (Williams) or Rps1b plants (L77-1863). Averages and standard errors are from 16 pairs of shots.
^{c}Ablation calculated as 1 − (Rps1b ratio)/(rps ratio) for ratios significantly different between rps and Rps1b.
^{d}p values comparing results from rps and Rps1b cultivars were calculated using the Wilcoxon rank sum test.
^{e}Ablations for KFLR and RFLQ were significantly different than wildtype (RFLR) with p < 0.001.

This example demonstrates that

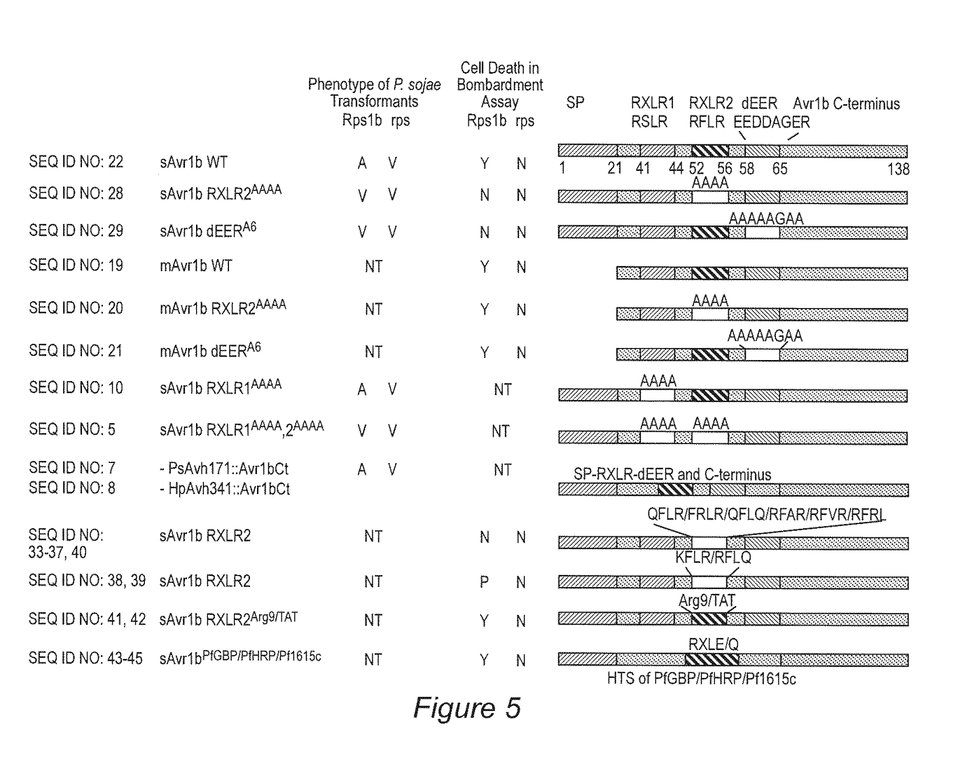

1) both the RXLR2 and dEER motifs of Avr1b are required for this protein to confer avirulence on P. sojae transformants (summarized in FIG. 5);
2) the RXLR2 and dEER motifs are not required to trigger an interaction with the Rps1b gene product when the Avr1b protein is synthesized in the soybean cytoplasm;
3) when Avr1b protein is directed to be secreted out of the soybean cell, the RXLR2 and dEER motifs are once more required for the protein to trigger an interaction with Rps1b, which is consistent with the motifs being required for the Avr1b protein to re-enter the soybean cell across the plasma cell membrane; 4) fusion of the RXLR and dEER region to GFP (SEQ ID NO: 46) enabled the isolated fusion protein to enter soybean root cells in the absence of the pathogen, but only if the RXLR and dEER motifs were both intact; and 5) RXLR-dependent entry of Avr1b does not require the presence of the pathogen. These observations lead to the conclusion that the RXLR and dEER motifs do indeed have the function of transporting avirulence proteins into host cells.

In addition, the data presented in this Example characterizes the RXLR2 and dEER motifs as follows: 1) arginine at position 1 and leucine at position 3 are essential for function of the RXLR motif. However, there is not a strong requirement for the arginine at position 4. Therefore by functional assays, the oomycete RXLR motif resembles the Plasmodium motif (RxL×D/E/Q) even more closely than previously noted; and 2) the amino acid sequences flanking the RXLR2 and dEER motifs are required in addition to the motifs themselves for the transit of Avr1b into soybean. Further, the region from residues 33 to 71 (19aa to the left of RXLR2 and baa to the right of dEER) were sufficient for protein translocation.

The Avr1b protein requires not only the RXLR motif itself, but also non-random surrounding sequences including the dEER motif. These surrounding sequences are not enriched in positive and hydrophobic residues, but instead are enriched in acidic and hydrophilic residues. Furthermore, our RXLR mutagenesis results show that the presence of basic and hydrophobic residues is not sufficient for RXLR function; instead the order of the amino acid residues is very important, and very subtle mutations such as RFLR→RFVR or QFLR abolish function. Therefore, oomycete effectors may utilize a novel mechanism for translocation across the membrane, possibly involving host cell surface machinery (such as a receptor) that is more complex than just the phospholipid bilayer. The Plasmodium Pexel/VTF motif also requires surrounding sequences that are enriched in acidic and hydrophilic residues and is functionally interchangeable with the oomycete RXLR domain in both erythrocytes and in soybean tissue (this study). Thus oomycetes and Plasmodium both may target host cell surface machinery that is common to plants and vertebrate animals but different than that targeted by animal PTDs. The targeted machinery, if common, must not only be very ancient, but also must serve an irreplaceable function in the host organisms since it must have been preserved against strong negative selection pressure resulting from exploitation by the pathogens.

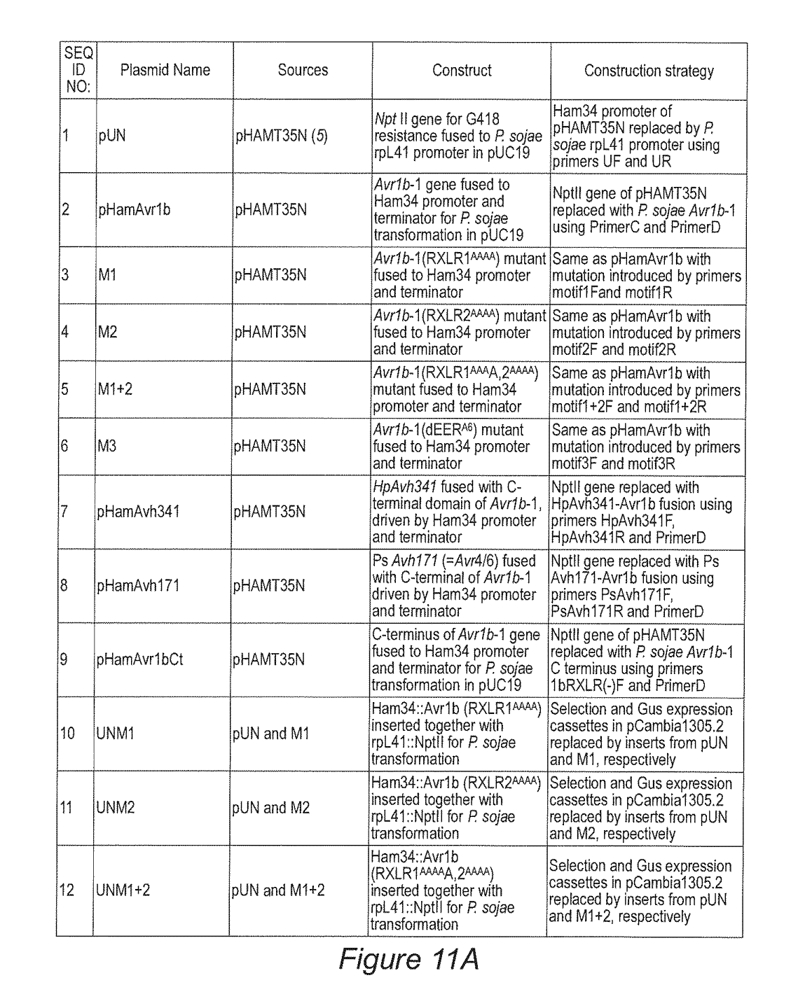

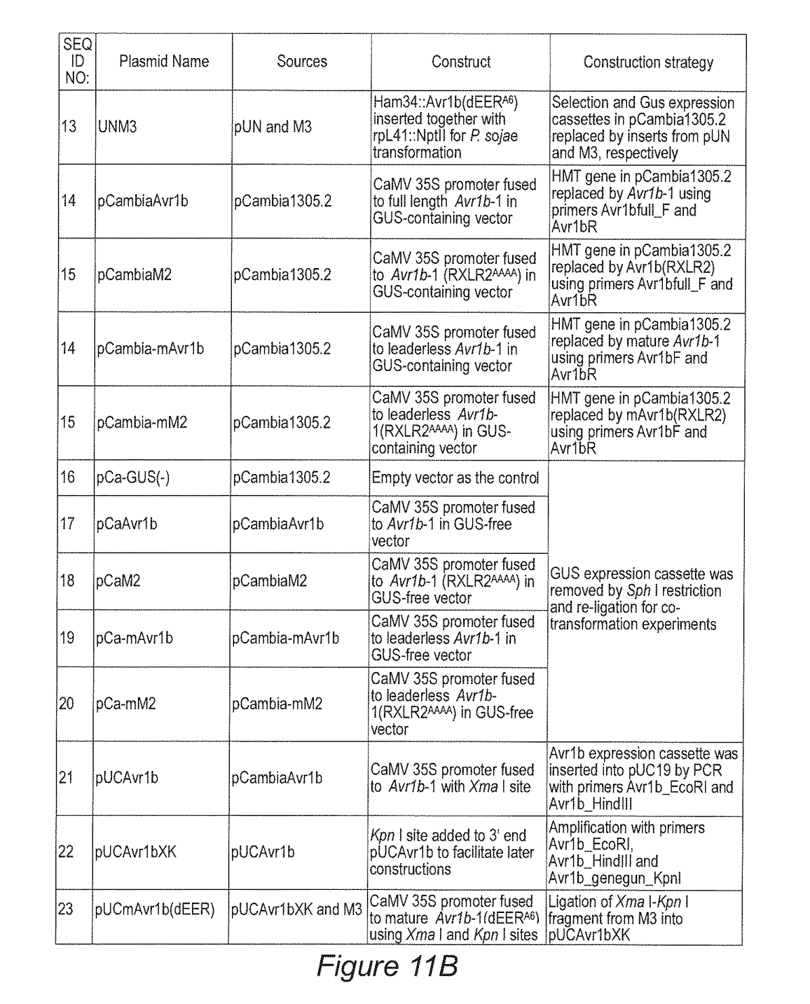

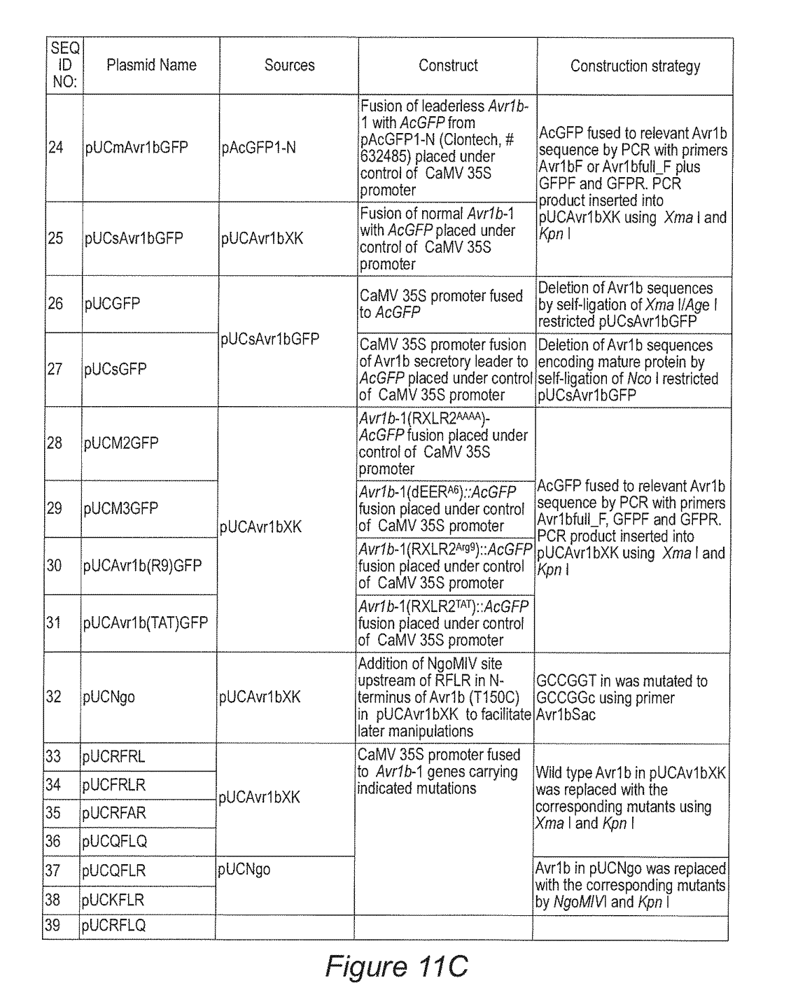

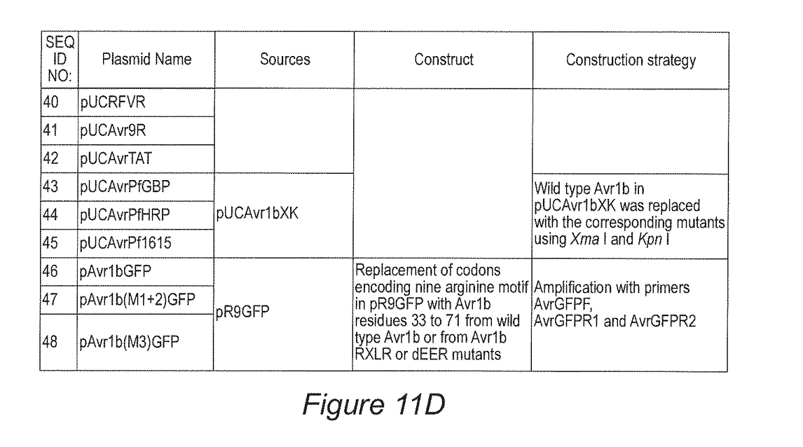

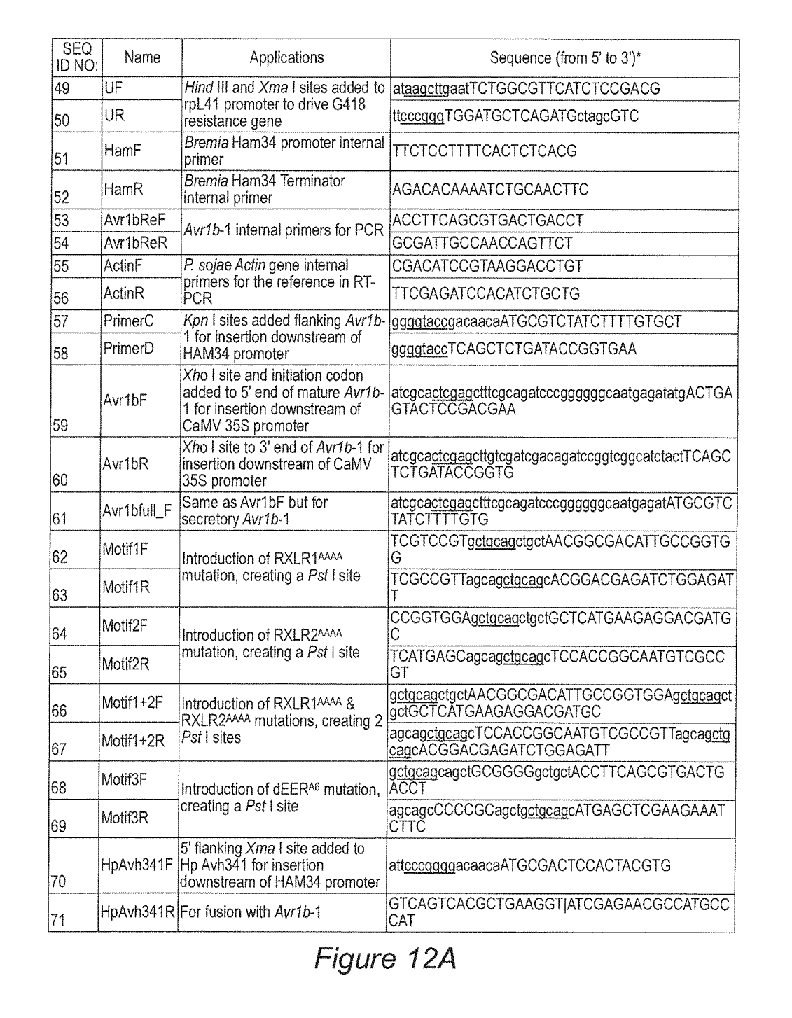

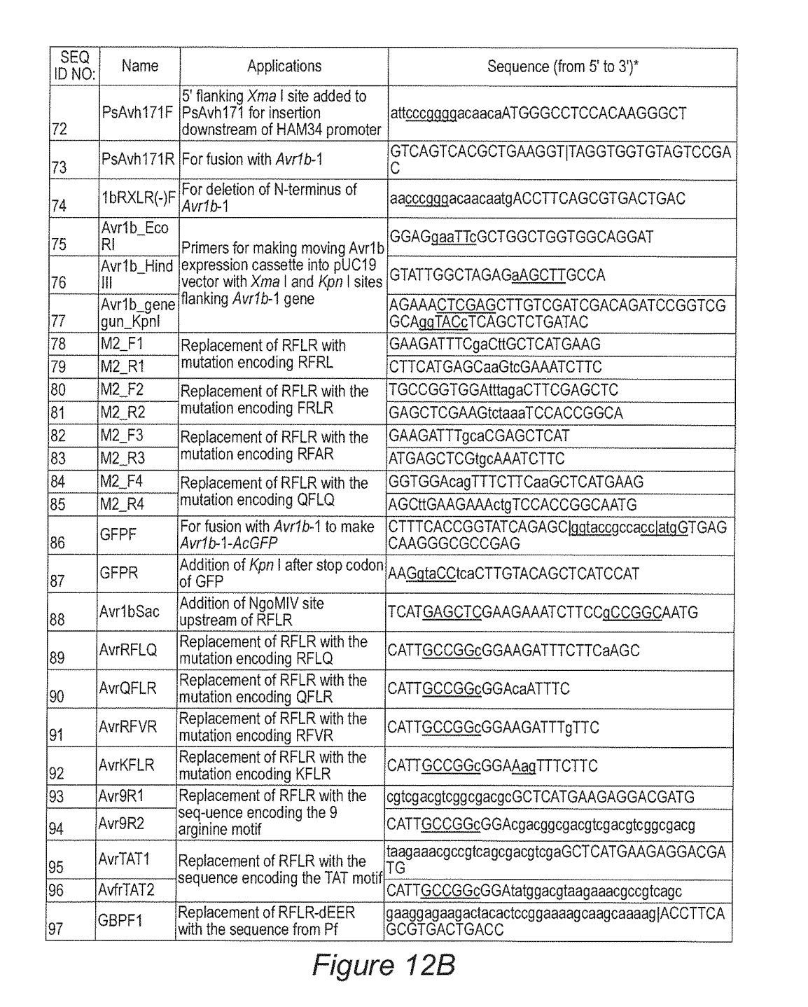

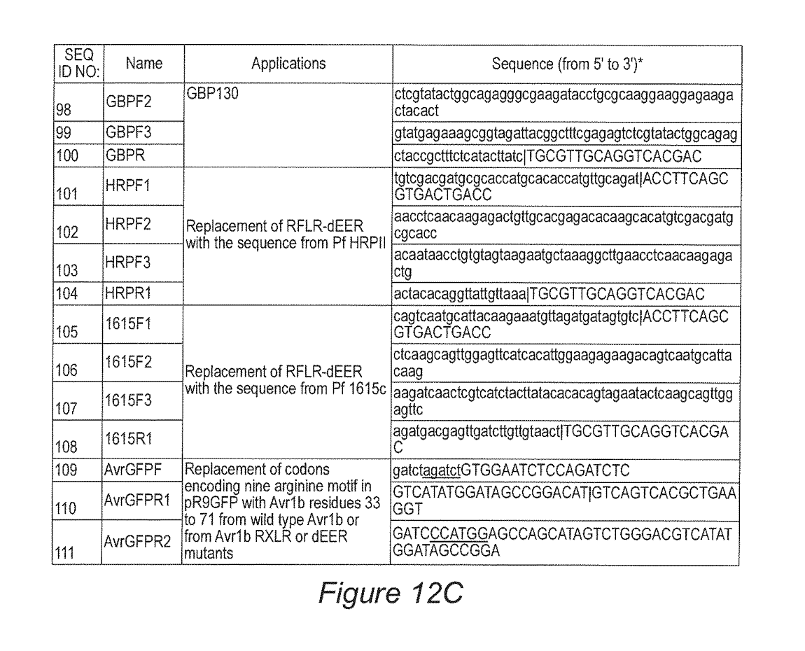

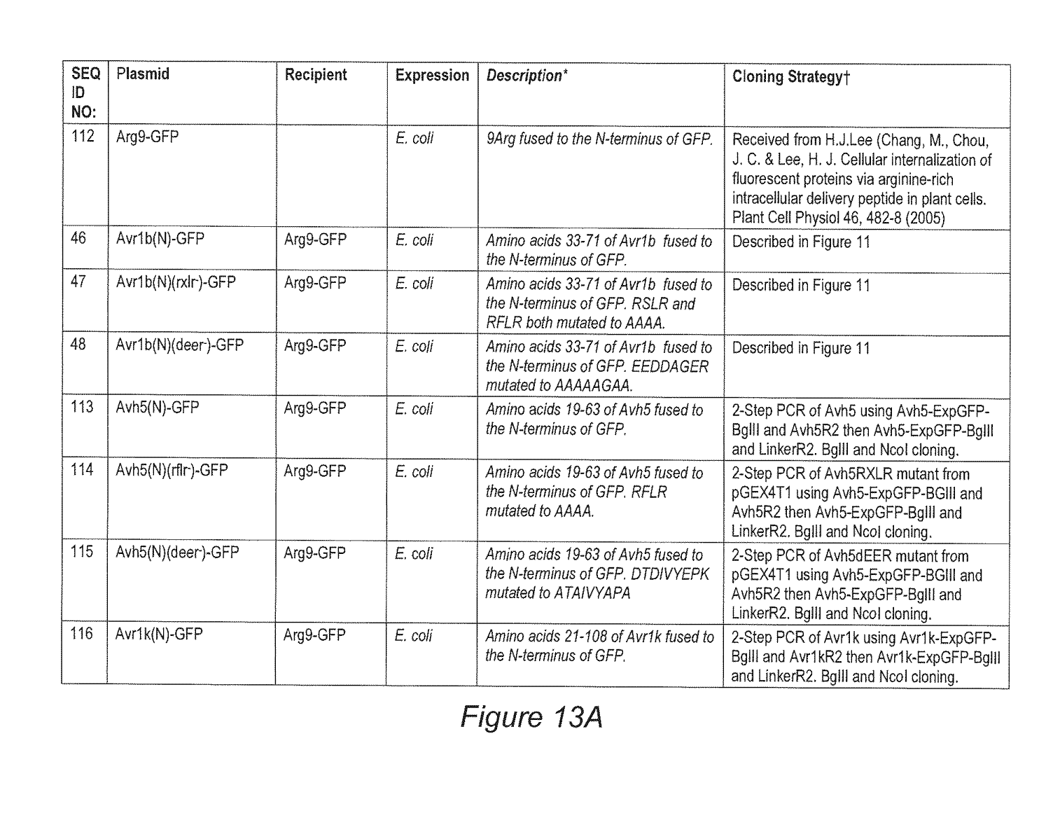

These results do not indicate which specific flanking sequences are required. However, HMMs constructed from the 10 amino acid residues flanking the upstream and downstream sides of all P. sojae and P. ramorum Avh RXLR motifs, clearly separated the RXLR motifs of functional avirulence proteins from RXLR motifs obtained by chance from real or permuted proteins sequences. These findings indicate that reliable bioinformatic searches for RXLR effector candidates should include the use of HMMs to evaluate the sequences flanking putative RXLR and dEER motifs. METHODS: Plasmids and oligonucleotides used in the study are depicted in tabular form as FIGS. 11A and B and FIGS. 12A and B, respectively.

P. sojae isolates and transformation: P. sojae isolate P7076 (Race 19) was routinely grown and maintained on V8 agar). The P. sojae transformation procedure was described by Dou et al (Dou, D., Kale, S. D., Wang, X., Chen, Y., Wang, Q., Wang, X., Jiang, R. H. Y., Arredondo, F. D., Anderson, R., Thakur, P., McDowell, J., Wang, Y., and Tyler, B. M. (2008) Plant Cell 20(4), 1118-1133).
Characterization, of P. sojae transformants: P. sojae transformants were selected that grew well on V8 medium with 50 μg/ml G418, and were cultured in V8 liquid medium for 3 days. The mycelia were harvested, frozen in liquid nitrogen and ground to a powder for DNA or RNA extraction. Genomic DNA was isolated from mycelium using known techniques. DNA samples were quantified using a Nanodrop ND-1000 spectrophotometer (Thermo Scientific). The presence of Avr1b-1 transgenes was verified by PCR amplification from 100 ng genomic DNA using a program of 94° C. for 2 min, 30 cycles of 94° C. for 30 s, 56° C. for 30 s, 72° C. for 30 s, and 72° C. for 5 min with primers of HamF and HamR (TS1). All the transformed P. sojae were double-checked by Pst I restriction and/or sequence. RNA was extracted from each sample using RNeasy Plant Mini Kit (QIAGEN, cat #74904) with β-mercaptoethanol added buffer RLT and genomic DNA was removed using RNase-Free DNase (QIAGEN, cat #79254) according to the manufacturer's recommendations. RNA was quantified using a Nanodrop ND-1000 spectrophotometer. Avr1b-1 transgene transcription was verified by RT-PCR using the internal primers, Avr1bReF and Avr1bReR (TS1) and P. sojae actin was used as the reference.
Phenotypic assays for avirulence: Avr1b phenotypic expression was assayed using soybean cultivars HARO(1-7) (rps), Haro13 (Harosoy background, Rps1b), Williams (rps) and L77-1863 (Williams background, Rps1b). Seedlings were grown in the greenhouse or in a growth chamber (Percival AR-36L) with a program of 24° C. at daytime and 22° C. at night with a 14 hr day length under fluorescent light (250 μmol photons s-1m-2). The virulence of each transformant was evaluated using hypocotyl inoculation. 1-2 days after the first primary leaf appeared, the hypocotyl of the soybean was wounded with a short incision and the incision was inoculated with a small piece of V8 agar cut from the edge of a 3 day old colony. Thereafter, the plants were incubated in a growth chamber under the conditions described above. The numbers of dead and surviving plants were counted 4 days after inoculation, and summed over 2-5 replicates. The differences between the numbers of surviving plants from rps and Rps1b cultivars were compared using Fisher's exact test. Only the transformants producing a significant difference between rps and Rps1b cultivars were judged as avirulent.
Particle bombardment assays: Particle bombardment assays were carried out using a double-barreled extension of the Bio-Rad He/1000 Particle Delivery System ((Dou, D., Kale, S. D., Wang, X., Chen, Y., Wang, Q., Wang, X., Jiang, R. H. Y., Arredondo, F. D., Anderson, R., Thakur, P., McDowell, J., Wang, Y., and Tyler, B. M. (2008) Plant Cell 20(4), 1118-1133). Analyzing the bombardment data as a ratio between the test and control shots improves the reproducibility of the measurements greatly. The avirulence activity of the Avr1b-1 constructs was measured as the reduction in the number of blue spots comparing the Avr1b-1+GUS bombardment with the GUS+control bombardment. For each paired shot the logarithm of the ratio of the spot numbers of Avr1b-1 to that of the control was calculated, then the log-ratios obtained from the Rps1b and non-Rps1b leaves were compared using the Wilcoxon rank sum test.
Bombardment assays of onion bulb cells with GFP constructs: Preparation of DNA-particle mixtures was as described above. 5 mm hemispherical layers of yellow and white onion bulbs were bombarded without the double barrel attachment under a 26 psi vacuum, using a rupture pressure of 1100 psi. The onion layers were incubated between 24-48 hr at 25° C., then viewed with a Zeiss Axioskop2 Plus microscope using a 480 nm filter for GFP fluorescence. Images were captured using a Qimaging Retiga 1300 Camera. To further confirm the GFP had been secreted out of the onion cells, plasmolysis was performed for 15 min in 0.8 M mannitol and cells were observed in a Zeiss LSM510 laser scanning confocal microscope (Jena, Germany) with an argon laser excitation wavelength of 488 nm.
RXLR-GFP Fusion Protein Expression and Purification: Residues 33 to 71 of Avr1b (VESPDLVRRSLRNGDIAGGRFLRAHEEDDAGERTFSVTD (SEQ ID NO: 46) including the RXLR1, RXLR2 and dEER motifs were fused to GFP, replacing the Arg9 encoding sequences in vector pR9GFP (SEQ ID NO: 112), called pR9 by Chang et al., (Chang, M., Chou, J. C. and Lee, H. J. (2005) Plant and Cell Physiology 46, 482-488). pR9GFP, which also adds an N-terminal His6 tag, was derived by Chang et al (2005) from Ptat-HA. C43(DE3) E. coli cells containing RXLR-GFP fusion constructs or pR9 were grown in 200 mL of LB containing ampicillin 100 μg/mL in a 1 L baffled flask shaken at 240 rpm at 37° C. until reaching an OD of 0.4, at which point the cells were induced by addition of 1 mL of 1M IPTG (final [5 mM]). After 4 hours further growth at the same conditions, the cells were harvested by centrifugation at 4° C. and then stored at −20° C. Visual confirmation of GFP expression was noted by the green color of the bacterial cell pellet. To extract the GFP fusion proteins, cells were thawed on ice for 20 min then 4 mL of lysis buffer (50 mM NaH₂PO₄, 300 mM NaCl, 10 mM imidazole, pH 8.0) were added per 1 g of wet cell weight. Lysozyme (Sigma-Aldrich, cat# L6876) was added to a final concentration of 1 mg/mL then the suspension was incubated for 20 min on ice. Sonication (Branson sonifier 150D, with Double stepped micro tip, 3 mm) was done at 300 W at 15 sec bursts four times with 15 sec cooling periods between each burst. The lysate was centrifuged at 10,000×g for 30 minutes at 4° C., then the supernatant was transferred to a fresh tube and kept on ice until use. 5 μL of each sample was stored for SDS-PAGE analysis. Protein purification using Ni-NTA affinity chromatography was performed using the QiaExpressionist protocol. 2 mL of 50% Ni-NTA super flow slurry (Qiagen) was loaded on a column. The column was washed twice with 5 mL of wash buffer (50 mM NaH₂PO₄, 300 mM NaCl, 20 mM imidazole, pH 8.0). The protein sample was loaded onto the column and then the column was washed twice with 10 vol (10 mL) of wash buffer. The protein was eluted with 4 mL of elution buffer (50 mM NaH₂PO₄, 300 mM NaCl, 200 mM imidazole, pH 8.0) into 1 mL fractions. These fractions were pooled and concentrated to 3001 using a centrifugal protein concentrator (Amicon Centriplus Centrifugal Filter Device MWCO-3 kDa) at 13,500×g. The sample was then mixed with an equal volume of 50 mM MES buffer pH 5.8. The protein concentration was measured at 280 nm using a nanodrop spectrophotometer (ND-1000) and adjusted to 8 mg/mL. All purified GFP preparations fluoresced normally under UV illumination.
RXLR-GFP Fusion Protein Root Cell Transduction Assay: Root tips were cut into lengths of between 0.5 cm and 1 cm, and then were washed with water. Each root tip was completely submerged in 20 μL of the protein solution (8 mg/ml in 25 mM MES pH 5.8) in a eppendorf tube. The samples were incubated overnight at 28° C. (˜12 hours). The roots were then washed in 200 mL of water for 4 hours while shaken at 100 rpm on a rotary shaker. The roots were then viewed using a Zeiss LSM510 laser scanning confocal microscope with an argon laser excitation wavelength of 488 nm. For nuclear staining, the roots were stained with DAPI (4′,6-diamidino-2-phenylindole) (Sigma-Aldrich cat# D8417) and viewed with a 405 nm filter.
Hidden Markof Model Analysis: By using the program HMMER 2.3.2 (Eddy, S. R. (1998). Profile hidden Markov models. Bioinformatics 14, 755-763; and website located at hmmer.janelia.org), an HMM was built from the full set of 765 high quality candidate effectors identified from the P. sojae and P. ramorum genomes by Jiang et al (Jiang, R. H. Y., Tripathy, S., Govers, F. & Tyler, B. M. Proc. Natl. Acad. Sci. USA 105, 4874-4879 (2008), using the 10 amino acids on the left side of each RXLR motif together with the 10 amino acids on the right side each RXLR motif. The same procedure was used to build an HMM from a curated list of 191 high quality candidate effectors from Hyaloperonospora parasitica developed at the H. parasitica genome annotation jamboree in August 2007 and available at pmgn.vbi.vt.edu. To estimate the significance of HMM scores, all proteins (1240) with a predicted N-terminal signal peptide (SP) and the string RXLR located between 30 and 60 amino acids after the SP cleavage site were obtained by translating the genome sequences of P. sojae and P. ramorum in all reading frames. The sequences of all the putative secreted proteins were permuted (other than the signal peptide) and RXLR-containing sequences were again identified; 639 of the permuted proteins had RXLR strings, indicating that about 639 of the 1240 detected RXLR motifs could be expected by chance. The distributions of HMM scores the set of 1240 real proteins, the 639 permuted proteins and the 765 curated proteins were then calculated. The frequency that a permuted protein received a score between 0 and 5.0 was 0.044. The frequency that a permuted protein received a score better than 5.0 was 0.018.
Accession Numbers The sequences reported herein have been deposited in the GenBank database, namely Hp Avh341 (EF681127). Accession numbers for sequences already in GenBank are Ps Avr1b-1 (AAM20936), Ps Avr4/6 (ABS50087), Pi Avr3a (CA172345); Hp Atr1 (AY842877), Hp Atr13 (AY785301).

### Example 2

### Effector Host-Targeting Signals of Eukaryotic Pathogens Bind Phosphoinositides or Phosphatidic Acid

Pathogens of both plants and animals produce effectors and/or toxins that act within the cytoplasm of host cells to suppress host defenses and cause disease. Effector proteins of oomycete plant pathogens utilize N-terminal motifs, RXLR and dEER, to enter host cells, and a similar motif, Pexel (RxLxE/D/Q), is used by Plasmodium effectors to enter erythrocytes. This Example shows that effectors of fungal plant pathogens contain functional variants of the RXLR and dEER motifs, and that the oomycete and fungal RXLR and dEER motifs, as well as the Plasmodium Pexel motifs, are responsible for binding of the effectors to phosphatidyl-inositol-3-phosphate (PI-3-P) and/or phosphatidyl-inositol-4-phosphate (PI-4-P). Stimulation of host cell entry by PI-4-P, and inhibition by inositol 1,4 diphosphate suggest that phosphoinositide binding mediates cell entry. All the effectors could also enter human cells, suggesting that phosphoinositide-mediated effector entry may be very widespread in plant, animal and human pathogenesis.

Oomycete RXLR and dEER Domain Binds Phosphoinositides

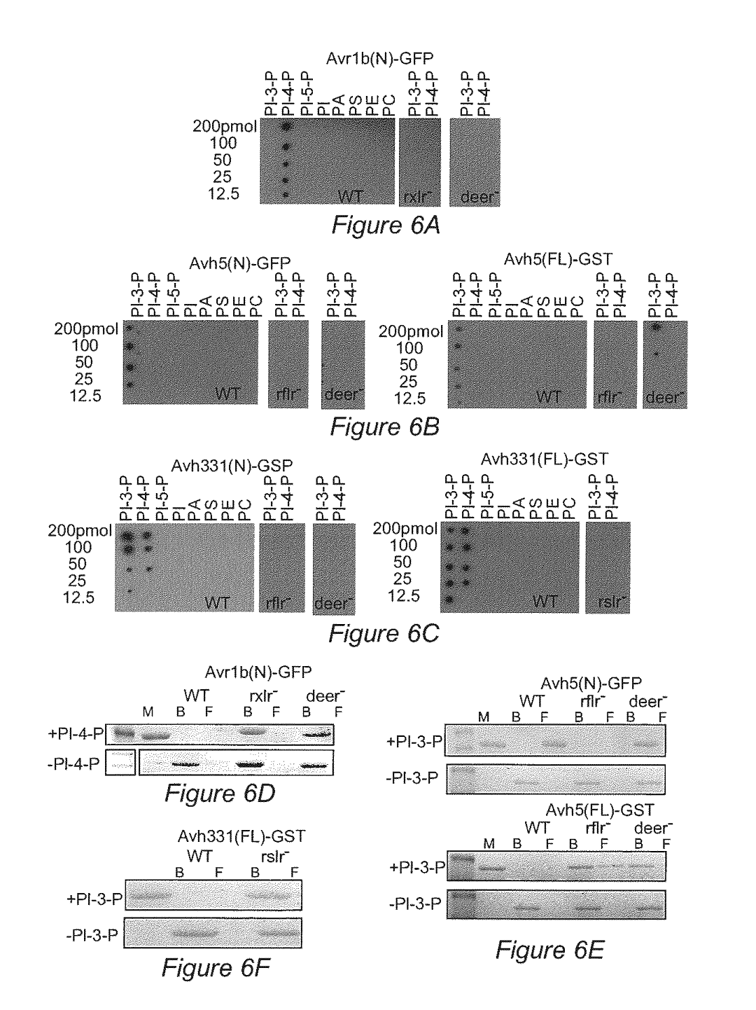

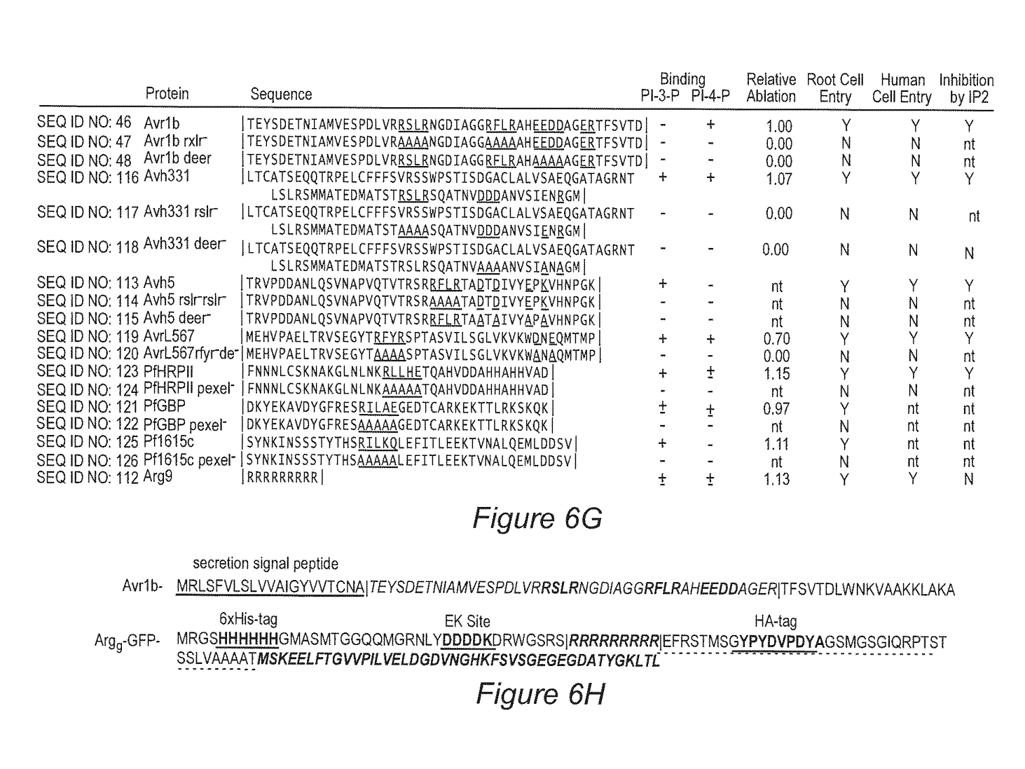

The RXLR and dEER domain of P. sojae Avr1b enables translocation of green fluorescent protein (GFP) into plant cells without any pathogen-encoded machinery (see Example 1), and the same is true for two additional bioinformatically predicted effectors, Avh5 (SEQ ID NO: 129) and Avh331 (SEQ ID NO: 127). In these experiments, accumulation of the GFP fusion proteins inside the cells was confirmed by the accumulation of GFP within the nuclei of the cells (a natural property of GFP), and by plasmolysis experiments. In principle, a cell entry domain could bind either a (glyco)protein or (glyco)lipid receptor. After noting that beta-type phosphatidylinositol-4-phosphate kinases from rice and Arabidopsis contained a PI-4-P binding domain consisting of 14 and 11 tandem RXLR and dEER motifs respectively, experiments were conducted to test whether oomycete the RXLR and dEER domain could bind phosphoinositides. An array of 8 different lipids found in plant cell membranes were spotted in decreasing amounts onto a Hybond-C extra membrane. Then the membrane was probed with GFP fused to the N-terminal RXLR and dEER domains of Avr1b (SEQ ID NO: 46), Avh331 (SEQ ID NO: 116) or Avh5 (SEQ ID NO: 113). FIG. 6 shows that the Avr1b- and Avh331-GFP fusions bound to PI-4-P while Avh331- and Avh5-GFP fusions bound to PI-3-P. Alanine substitutions mutations in either the RXLR or the dEER motif of any of the three fusions abolished binding, just as they abolished entry into soybean root cells. Fusions of full-length Avh5 (SEQ ID NO: 129) or Avh331 (SEQ ID NO: 127) proteins at their N-termini to glutathione-S-transferase (GST) could also bind the same phosphoinositides as just their N-terminal domains fused at their C-termini to GFP and binding by the full length proteins also required intact RXLR and dEER motifs (FIG. 6) (full-length Avr1b could not be produced in E. coli).

To independently confirm binding of the effector RXLR and dEER domains to the phosphoinositides, the binding of the fusion proteins to liposomes composed of phosphatidyl-choline (PC) and phosphatidyl-ethanolamine (PE) was tested. In the absence of phosphoinositides, neither the effector N terminus-GFP fusion proteins nor the full length GST-effector fusion proteins bound the liposomes (FIG. 6). However, when either PI-3-P or PI-4-P were included, all the fusion proteins bound to the liposomes. In every case, when any of the RXLR or the dEER motifs were mutated by alanine substitutions, the mutant fusion proteins lost their ability to bind the liposomes (FIG. 6).

### Identification of Fungal Effector Translocation Domains

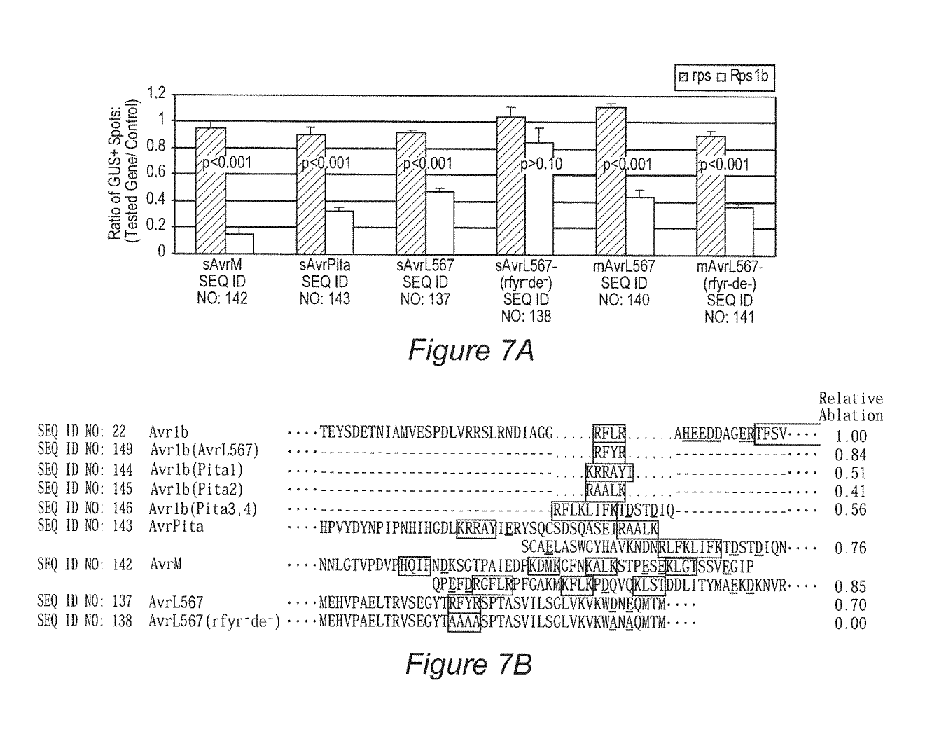

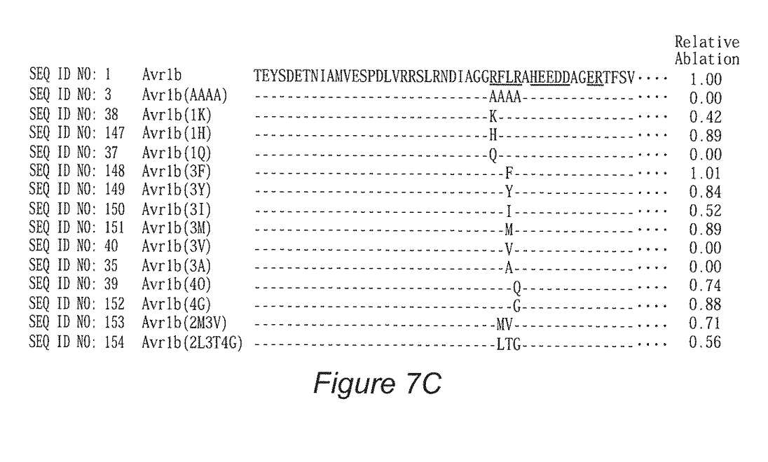

To test whether fungal effectors contain N-terminal cell entry domains, N-terminal segments from the fungal effectors AvrL567 (SEQ ID NO: 138) and AvrM (SEQ ID NO: 142) of M. lini and from AvrPi-ta (SEQ ID NO: 143) of M. oryzae were fused to the C-terminus of Avr1b, in the presence of the Avr1b secretory leader, then tested the fusions in a particle bombardment cell re-entry assay that measures the ability of a motif to carry an Avr1b reporter protein back into soybean leaf cells after secretion. FIG. 7A shows that all three fungal N-terminal segments had significant ability to deliver Avr1b back into soybean leaf cells.

Since the fungal effectors contained no obvious RXLR or dEER motifs, we decided to define experimentally the range of residues within the RXLR motif of Avr1b that could permit cell entry, using the particle bombardment cell re-entry assay. The results revealed that lysine (K) or histidine (H) but not glutamine (Q) could replace the arginine at position 1 in the motif, that any large hydrophobic residue (isoleucine, I; methionine, M; phenylalanine, F; tyrosine, Y) could replace the leucine at position 3, albeit with varying efficiencies, but valine (V) and alanine (A) could not. At position 4, all residues tested (lysine, K; glutamine, Q; glycine, G) allowed function. Furthermore, the presence of either an L or M residue at position 2 could substitute for a large hydrophobic residue at position 3.

Using this information, one, seven and four potential cell entry motifs were identified in N-terminal regions of AvrL567 (SEQ ID NO: 138), AvrM (SEQ ID NO: 142) and AvrPi-ta (SEQ ID NO: 143), respectively (FIG. 7B). The single motif in AvrL567, RFYR, had a particularly good match to the oomycete RXLR motif, and RFYR had already been shown to be functional (FIGS. 7B and 7C). Four of the candidate motifs in the AvrPi-ta N-terminus, including two that overlapped at one residue (RFLK and KLIFK (SEQ ID NO: 146)), were tested for cell entry activity by substituting them for the RXLR motif of Avr1b. The two single motifs and the overlapping pair were all active in the cell re-entry assay (FIG. 7B).

The N-terminus of AvrL567 was subjected to further analysis by mutagenesis and root cell entry assays. Alanine substitutions in the RFYR motif and in two downstream acidic residues that might act as a dEER motif (FIG. 7B) (SEQ ID NO: 139), abolished the activity of the AvrL567 N-terminal domain in the particle bombardment cell re-entry assay (FIGS. 7A and B). To confirm the cell entry activity of the AvrL567 N-terminal domain, it was fused to GFP (creating AvrL567(N)-GFP (SEQ ID NO: 119)), with and without (SEQ ID NO: 120) the alanine substitutions, and then the fusion proteins were tested for cell entry in the soybean root uptake assay. The GFP-fusion with the intact AvrL567 N-terminus (SEQ ID NO: 119) efficiently accumulated in the root cells, including the nuclei, whereas the fusion with the mutated RFYR and acidic residues (rfyr-de-) (SEQ ID NO: 120) did not (not shown). Thus the RFYR motif and the two downstream acidic residues appear to act as a RXLR and dEER motif in M. lini AvrL567.

### Fungal and Apicomplexan Effectors Bind Phosphoinositides

Both filter binding and liposome binding were used to test whether the N-terminal domain of AvrL567 bound phosphoinositides. AvrL567(N)-GFP (SEQ ID NO: 119) bound PI-3-P in both assays. Binding of AvrL567(N)-GFP to PI-4-P was also be detected in the liposome assay though it is not as strong as to PI-3-P. Mutation of the RXLR and dEER-like motif to alanines (rfyr-de-mutant) (SEQ ID NO: 120) resulted in a loss of binding to the phosphoinositides in both assays.

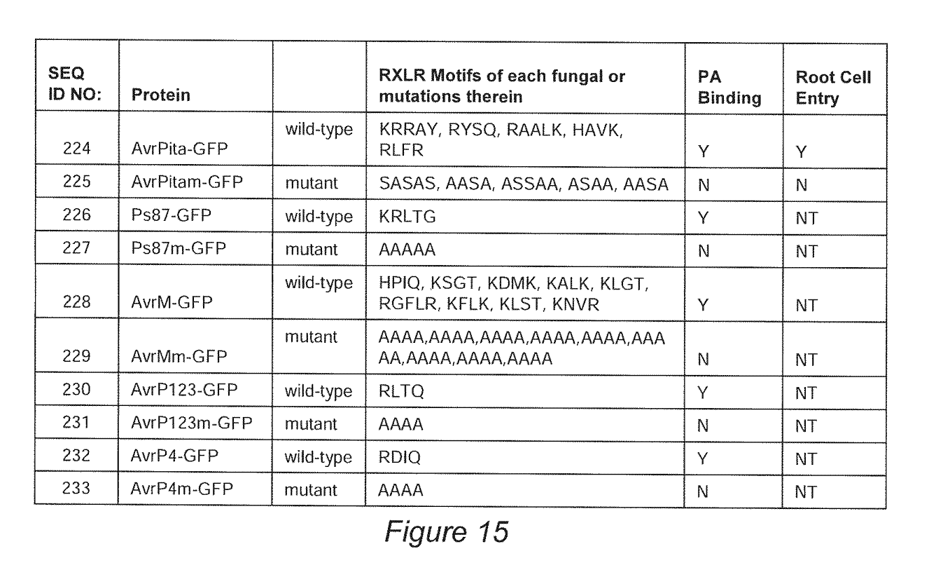

Filter binding assays were used to determine if five additional fungal effectors could bind phospholipids. The N-terminal sequences of the following effectors were fused to GFP: Magnaporthe grisea AvrPita (SEQ ID NO: 224), Puccinia graminis Ps87 (SEQ ID NO: 226); Melampsora lini AvrM (SEQ ID NO: 228); Melampsora lini AvrP123 (SEQ ID NO: 230); Melampsora lini AvrP4 (SEQ ID NO: 232). The results, summarized in tabular form in FIG. 15, showed that all five effectors bound phosphatidic acid. Mutations in RXLR-like motifs found in AvrPtia (SEQ ID NO: 225), Ps87 (SEQ ID NO: 227), AvrM (SEQ ID NO: 228), AvrP123 (SEQ ID NO: 230) and AvrP4 (SEQ ID NO: 232) all abolished binding to phosphatidic acid. In the case of AvrPita, the mutant protein (SEQ ID NO: 225) was unable to enter soybean root cells, whereas the wildtype protein (SEQ ID NO: 224) could enter root cells, suggesting that binding of AvrPita to phosphatidic acid was required to enter plant cells.

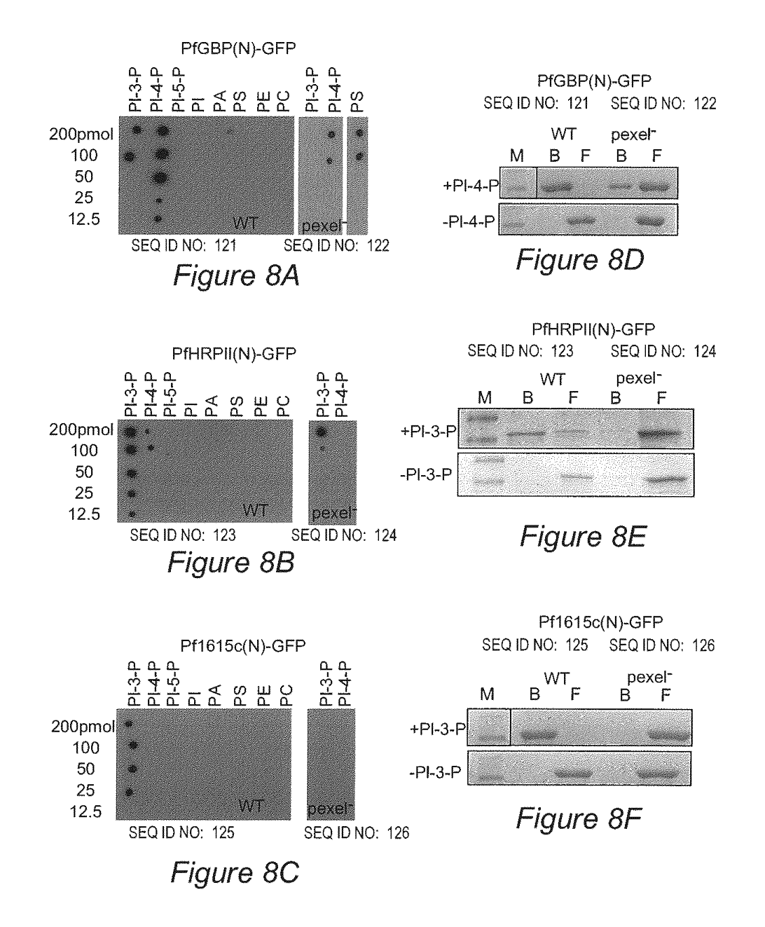

The host targeting signals (HTS) of three Plasmodium falciparum effectors, PfGBP (SEQ ID NO: 121), PfHRPII (SEQ ID NO: 123), and Pf1615c (SEQ ID NO: 125) can carry Avr1b into soybean leaf cells and onion bulb epidermal cells8. The three signals can also carry purified GFP into soybean root cells and this activity requires intact Pexel motifs. To test whether the three signals also could bind phosphoinositides, the HTS-GFP fusion proteins were tested using filter binding and liposome binding assays. The PfGBP HTS fusion (SEQ ID NO: 121) could bind PI-4-P and also, more weakly, PI-3-P (FIG. 8A). The PfHRPII HTS fusion (SEQ ID NO: 123) could bind PI-3-P, and also rather weakly, PI-4-P (FIG. 8B) The Pf1615c HTS fusion (SEQ ID NO: 125) could bind specifically to PI-3-P (FIG. 8C). Liposome binding assays confirmed binding of all the fusion proteins to PI-3-P or PI-4-P (FIGS. 8D-F). In both assays, alanine substitutions in the Pexel motifs of each effector abolished phosphoinositide binding (FIG. 8A-F) (SEQ ID NO: 122, 124, 126).

### Modulation of Effector Entry by Exogenous Phosphoinositides

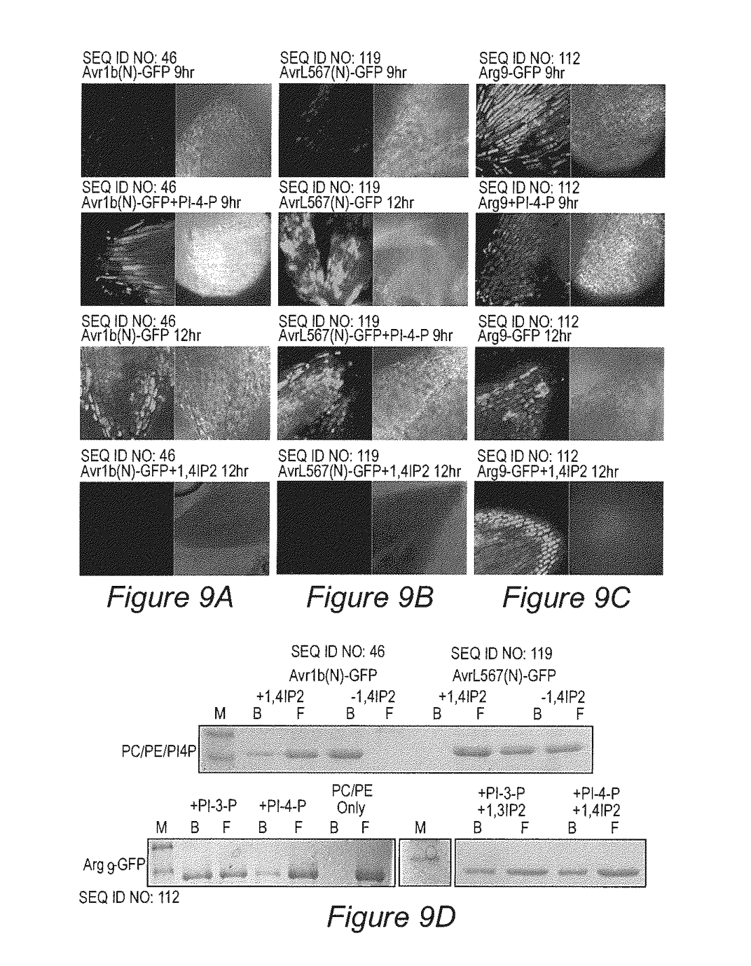

The binding of phosphoinositides to the effector cell entry domains suggested that these phospholipids might serve as a cell entry receptor in each case. Tomato cells secreted PI-4-P when stimulated by fungal xylanase, suggesting that free PI-4-P might exist in the plant apoplast24. Therefore, increasing the concentration of free phosphoinositide by exogenous addition might stimulate RXLR and dEER-mediated uptake. To test this hypothesis, a soluble form of PI-4-P, di-octanoyl-PI-4-P (250 μM), was mixed with the Avr1b GFP fusion, Avr1b(N)-GFP, for 30 min prior to exposure to soybean roots. After 9 hr, strong stimulation of Avr1b(N)-GFP uptake by PI-4-P was evident (FIG. 9A). The phospholipids, PI, PC and PE all did not stimulate uptake. Uptake of the fungal AvrL567(N)-GFP fusion (SEQ ID NO: 119) was also strongly stimulated by PI-4-P (FIG. 9B), even though it binds most strongly to PI-3-P.

A synthetic cell entry motif composed of nine-arginine residues (Arg9) (SEQ ID NO: 112) was previously shown to deliver Avr1b into soybean leaf cells and into onion epidermal leaf cells in particle bombardment cell re-entry assays. The motif could also enable uptake of purified GFP into soybean root cells8 and into maize and onion cells. The mechanism of uptake has been proposed to be a plant form of macropinocytosis. The Arg9-GFP fusion protein binds PI-3-P, PI-4-P and phosphatidyl serine, albeit weakly (FIG. 9D). FIG. 9C shows that di-octanoyl-PI-4-P does not stimulate uptake of the Arg9-GFP fusion protein in soybean root cells, suggesting that the stimulation by PI-4-P is specific to RXLR and dEER-mediated uptake. This conclusion is supported by the observation that exogenous PI-4-P did not promote the uptake of Avr1b(N)-GFP (SEQ ID NO: 46) and AvrL567(N)-GFP (SEQ ID NO: 119) proteins containing alanine substitutions in the RXLR and dEER motifs.

Inositol-1,4-diphosphate (IP2) represents the hydrophilic head-group of PI-4-P. Preincubation with 100 μM IP2 inhibited binding of Avr1b(N)-GFP (SEQ ID NO: 46) to PI-4-P-containing liposomes and could completely block binding of AvrL567(N)-GFP (SEQ ID NO: 119) to PI-4-P-containing liposomes, presumably via competitive inhibition. To test whether IP2 could block effector uptake in planta, which would imply that a PI-4-P-like molecule mediated uptake in planta, Avr1b(N)-GFP (SEQ ID NO: 46) or AvrL567(N)-GFP (SEQ ID NO: 119) was preincubated with 500 μM IP2 for 30 min prior to exposure to soybean roots. IP2 almost completely blocked uptake of both Avr1b(N)-GFP (FIG. 9A) (SEQ ID NO: 46) or AvrL567(N)-GFP (FIG. 9B) (SEQ ID NO: 119) into soybean cells. IP2 could not inhibit the binding of Arg9-GFP to liposomes (FIG. 9D) and uptake of Arg9-GFP (SEQ ID NO: 112) was completely unaffected by preincubation with IP2 (FIG. 9C), supporting the conclusion that IP2 specifically blocks RXLR and dEER motif-mediated protein uptake.

Effector Entry into Human Cells

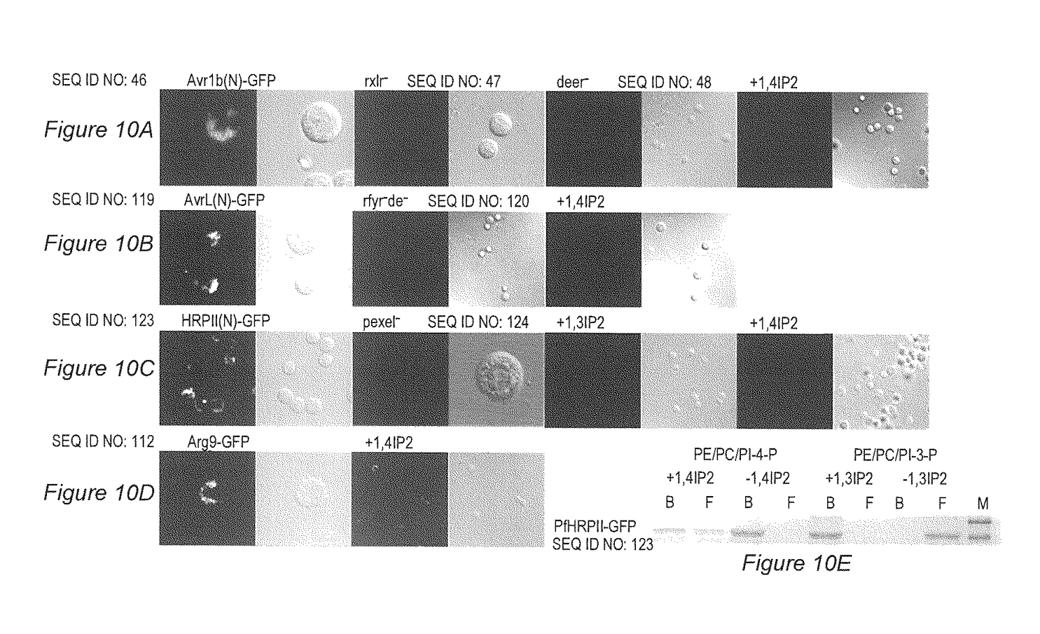

Phosphatidyl-inositol-phosphates are universally found in eukaryotic cells. Since a number of human and animal diseases are caused by fungi and oomycetes, as well as by apicomplexan parasites, we tested the possibility that RXLR and dEER motifs might mediate protein entry into human cells, using the human lung epithelial cell line A549 as a model. Avr1b(N)-GFP (FIG. 10A) (SEQ ID NO: 46), AvrL567(N)-GFP (FIG. 10B) (SEQ ID NO: 119) and PfHRPII(N)-GFP (FIG. 10C) (SEQ ID NO: 123) (SEQ ID NO: 60) could all enter the A549 cells, but entry did not occur if alanine substitutions were present in the RXLR or Pexel motifs of the proteins (SEQ ID NO: 47,48,120,124). In these experiments, accumulation of the GFP fusion proteins inside the cells was confirmed by the accumulation of GFP within vesicle-like structures within the cells, and by the fact that the cells were treated with protease (trypsin) prior to photographing. Protein accumulation was strongly inhibited in each case by 1,4-IP2, supporting the hypothesis that entry was mediated by phosphoinositide binding. Exogenous di-octanoyl-PI-4-P did not stimulate accumulation, suggesting that the availability of PI-4-P or other phosphatidylinositides in the growth medium was not limiting. Inositol 1,3 diphosphate (1, 3-1P2), the headgroup of PI-3-P, could also inhibit entry of PfHRPII(N)-GFP (FIG. 10C) (SEQ ID NO: 123), consistent with the observation that the protein bound PI-3-P more strongly than PI-4-P in the filter-binding assay.

Discussion: Two independent assays, namely filter-binding and liposome-binding, demonstrated that the N-terminus of all seven effectors tested could bind to either PI-3-P or PI-4-P, but not to PI-5-P nor to any other phospholipids tested. The primary structures of the RXLR and dEER effector domains do not resemble any known phosphoinositide binding domains. However, the binding of the pathogen RXLR and dEER domains to phosphoinositides is concordant with the binding of the RXLR and dEER domains of rice and Arabidopsis (3-type PI-4-kinases. In the oomycete effector proteins, the dEER motif is variably spaced from the RXLR motif, so if residues from both motifs contact the phosphoinositide head group, the protein must fold so as to bring the two motifs into proximity. The three dimensional structure of the RXLR and dEER or Pexel domain is not yet available for any oomycete or apicomplexan effector proteins, respectively. However, the crystal structure of AvrL567 has been determined. In this structure, the RFYR motif adopts a beta-stranded conformation on the surface of the protein. It will be interesting to determine if the structure of AvrL567 changes in solution in the presence of a phosphoinositide.

The stimulation of protein entry into soybean root cells by PI-4-P and the inhibition of entry by IP2 together support the hypothesis that binding to phosphoinositides mediates entry of these pathogen effectors into plant cells. Similar findings with a human lung epithelial cell line suggest the possibility that effectors of oomycetes and fungi that infect humans and other animals might enter host cells via a similar mechanism. This mechanism appears to be different than reported for other peptides with cell entry activity because entry of those other peptides is not dependent on phosphoinositides.

These data demonstrate that several fungal effectors contain N-terminal domains that are capable of carrying Avr1b into soybean leaf cells. Within these domains are RXLR-like motifs that can replace the Avr1b RXLR motif in carrying reporter proteins into host cells. In the one case tested in detail so far, AvrL567, the motif RFYR was identified as necessary for the activity of the N-terminal cell entry domain, and for the binding of the domain to PI-3-P. The mutagenesis survey of the Avr1b RXLR motif and the diverse functional motifs found in the fungal effectors together suggest that a wide diversity of RXLR-like sequences support binding of phosphoinositides. Bioinformatic screens with the highly redundant motif suggested by the data identifies huge numbers of matches, most of which are likely spurious as judged by searches of permuted protein sequences. Thus, it is very likely that there are additional requirements for phosphoinositide binding. Both the RXLR and dEER motifs of Avr1b and the Pexel motifs of Plasmodium effectors are insufficient by themselves to facilitate cell entry; in both cases flanking sequences are required.

Other than in apicomplexan parasites, eukaryotic pathogens of humans and other animals have not been reported to produce effector proteins that can cross host membranes into the cytoplasm of host cells. The finding that phosphoinositide-binding effectors from oomycete and fungal plant pathogens can cross the membranes of human cells predicts that oomycete and fungal pathogens of humans and other animals may also utilize this mechanism to debilitate their hosts. Possible examples include oomycete pathogens of marine animals from the genera Saprolegnia and Aphanomyces, extracellular fungal pathogens such as Pneumocystis carinii, Coccidioides iinnzitus and Aspergillus fumigatus, and intra-phagosomal fungal pathogens of humans such as Cryptococcus neoformans and Histoplasma capsulatum.

The binding of phosphoinositides or other polar lipids to effector cell entry domains from diverse kingdoms will provide a powerful biochemical tool for screening or directly isolating new candidate effector proteins from all classes of microbes. It may also enable detection of phosphoinositide-binding plant proteins (or other polar-lipid-binding proteins) that can traffic through the apoplast and enter into target cells to transduce signals. Some precedents for such proteins already exist, such as the Drosophila antennapedia transcription factor that can move from cell to cell via an arginine-rich cell entry motif.

Understanding the role of phosphoinositides and other polar lipids in pathogen effector entry also opens the possibility of targeting cell entry domains for preventative or therapeutic intervention in both agriculture and medicine. The finding that 1P2 can block effector entry into both plant and human cells provides a proof-of-concept for this approach.

### Methods Summary

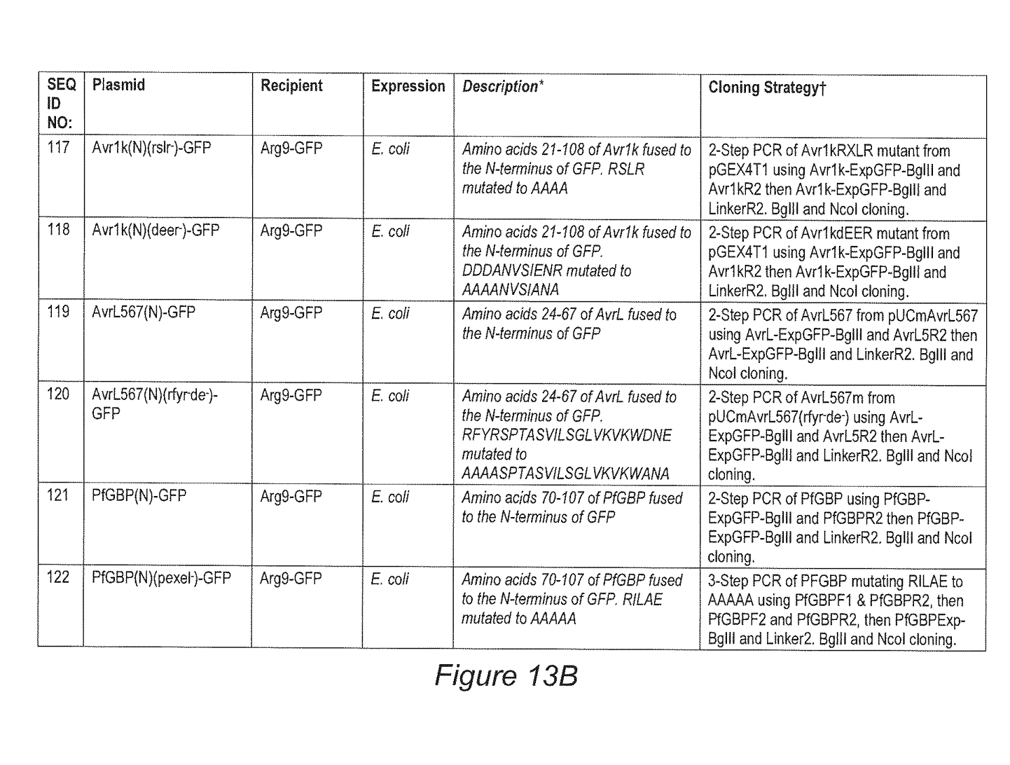

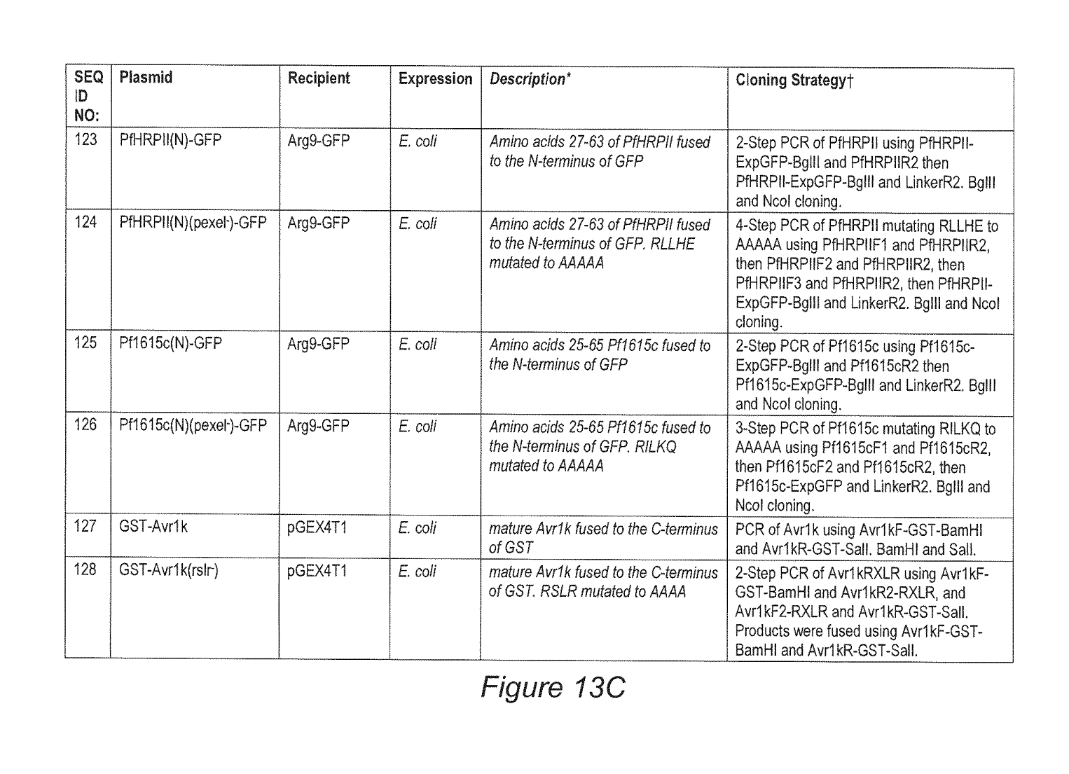

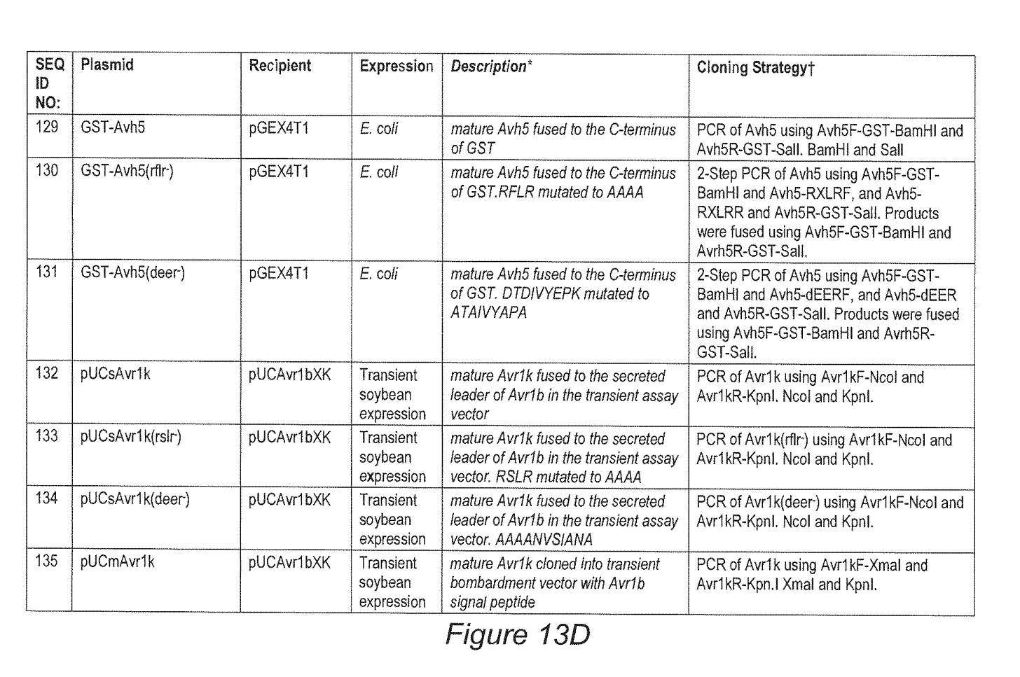

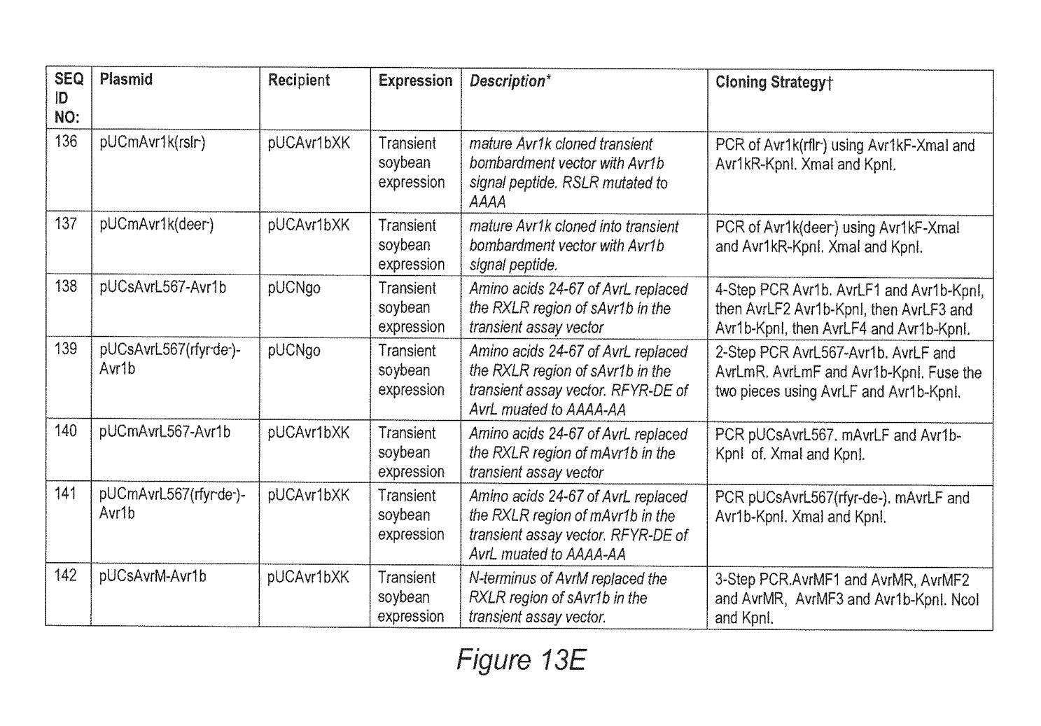

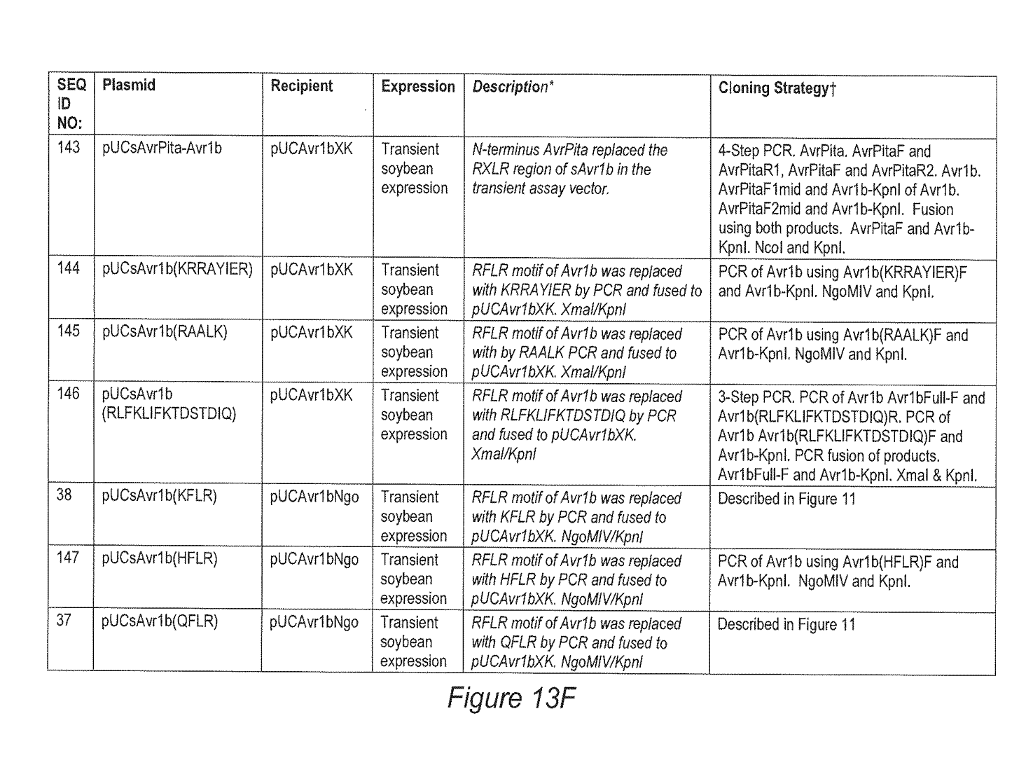

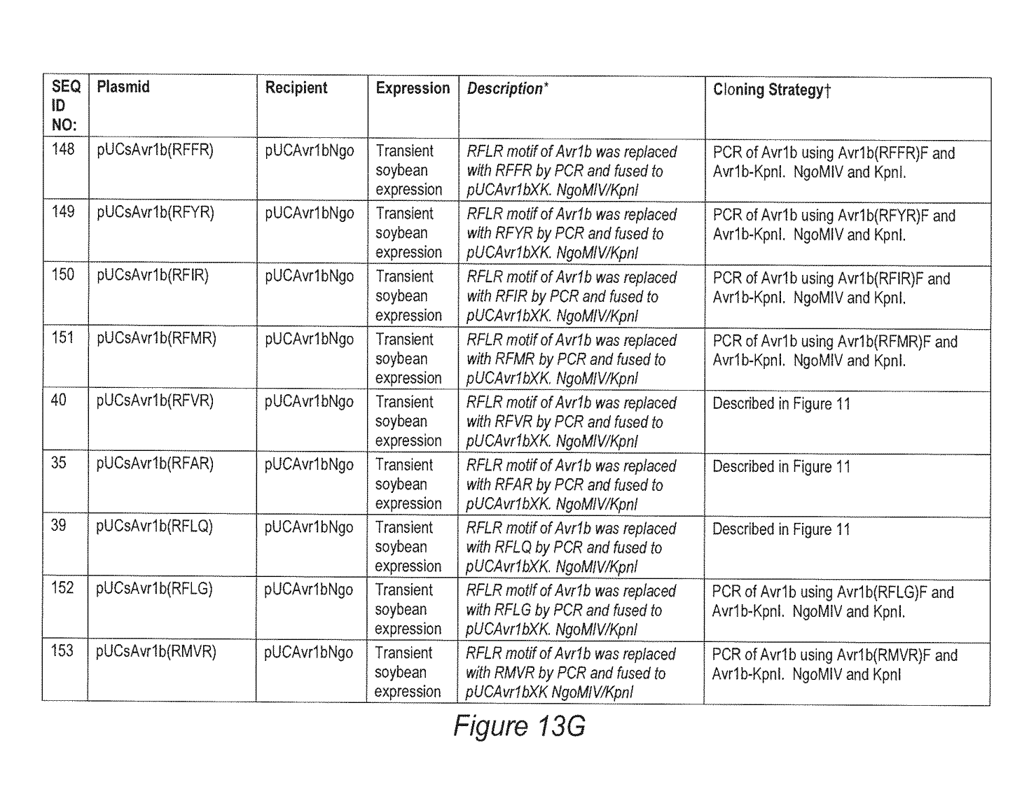

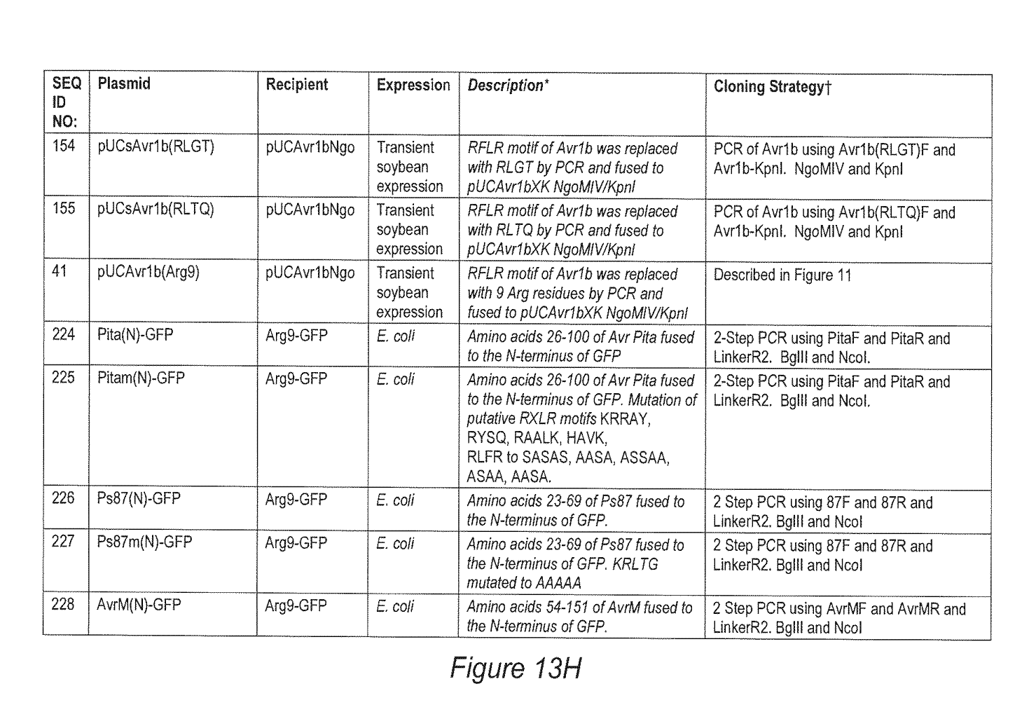

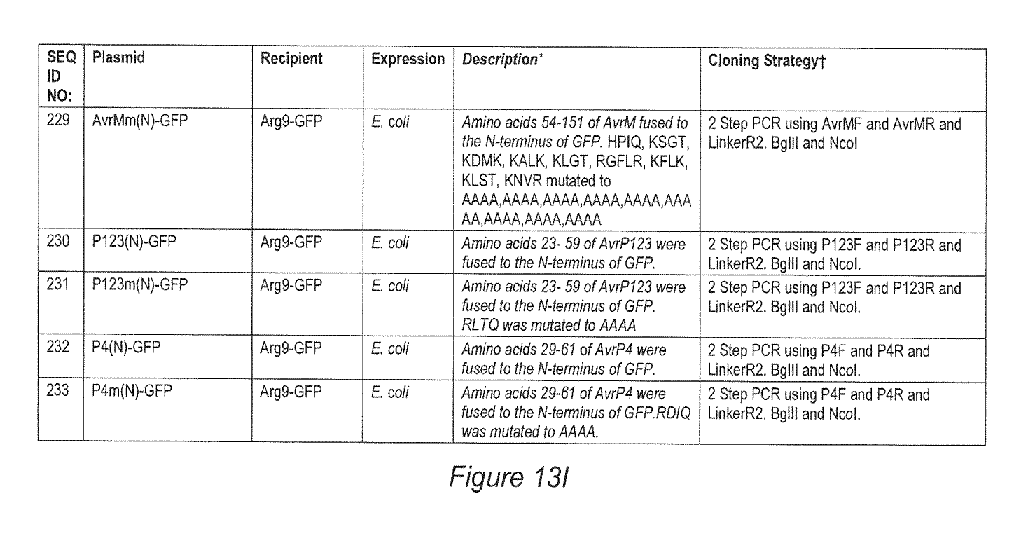

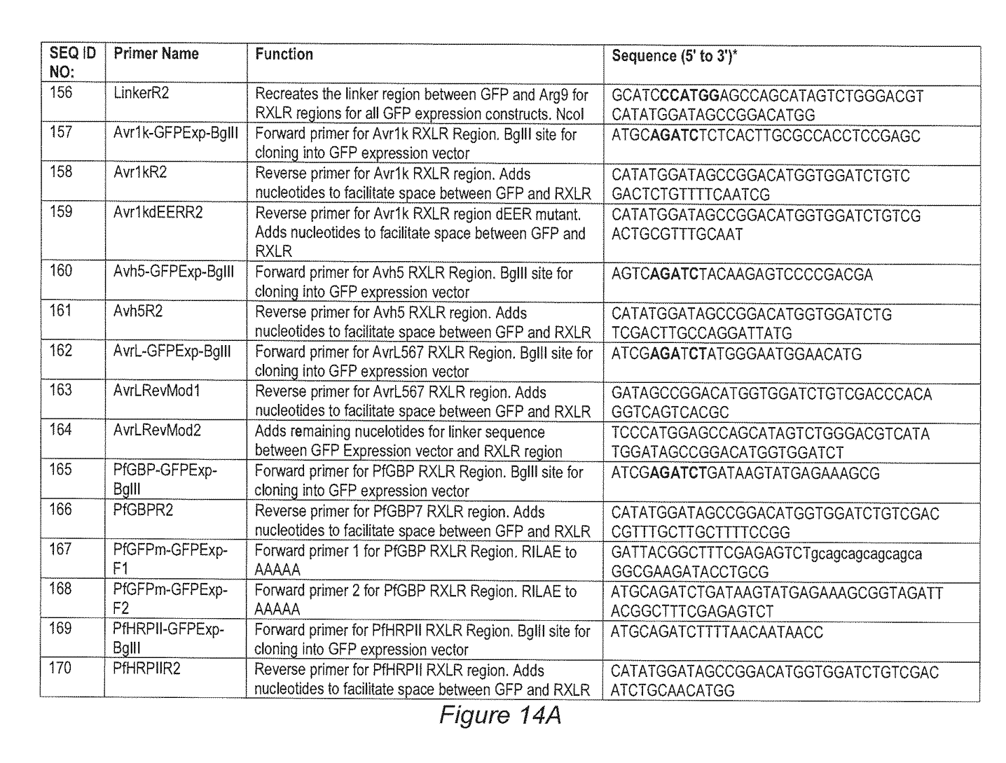

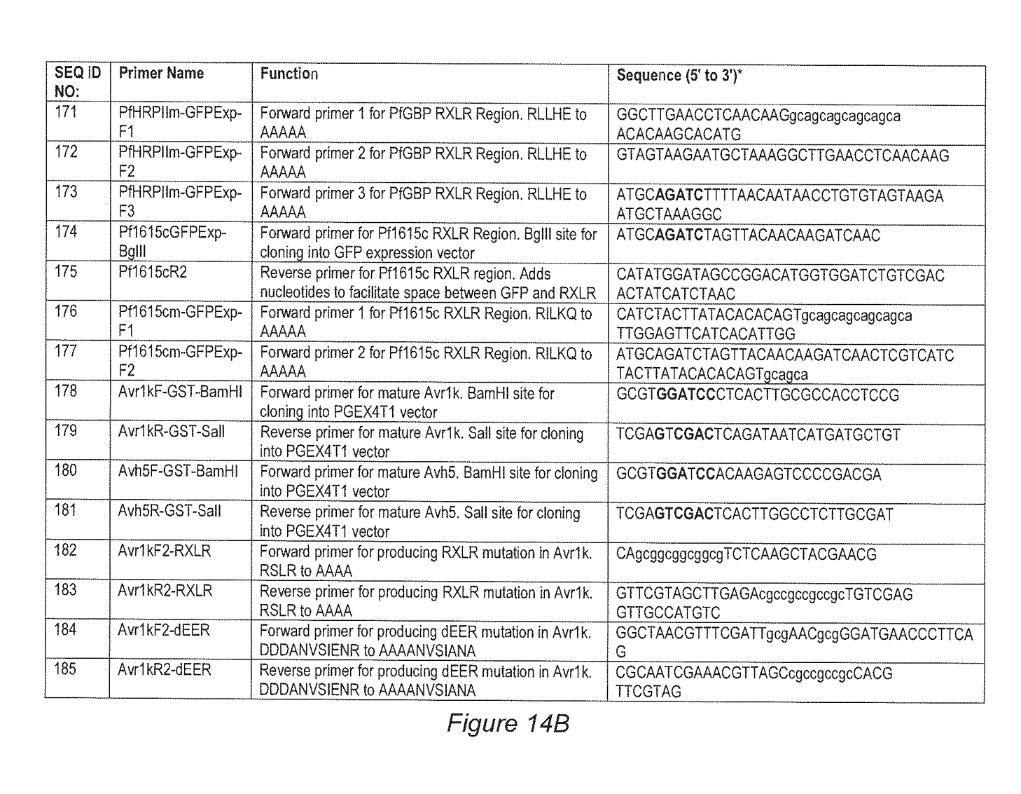

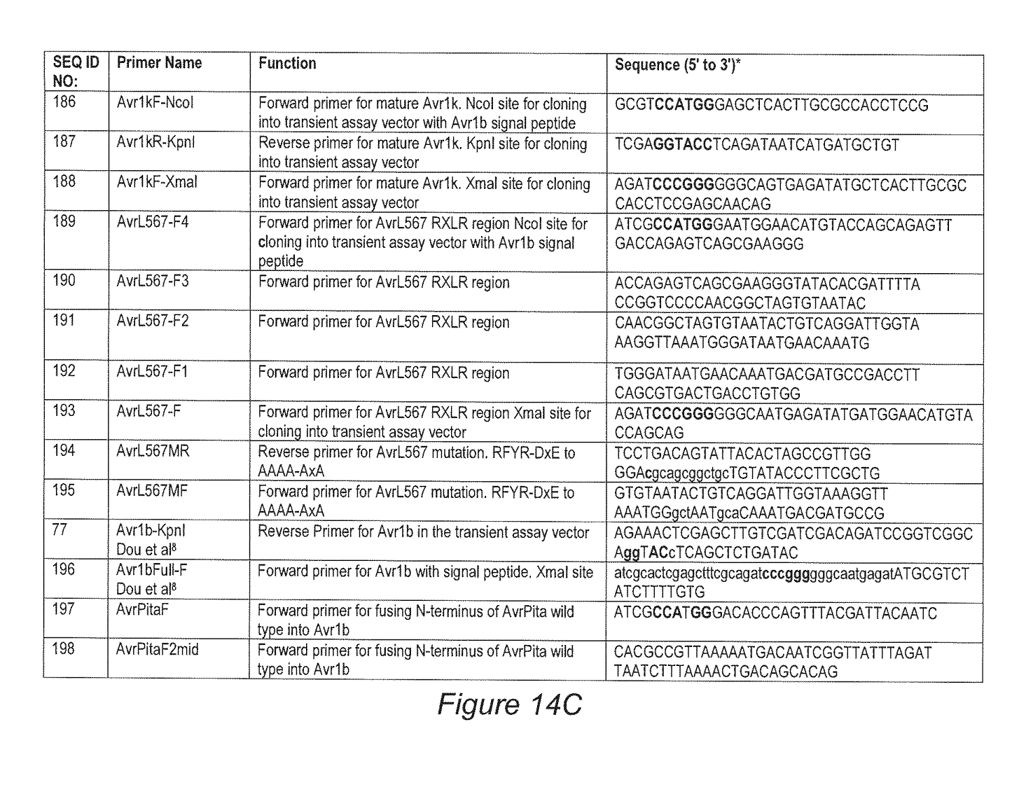

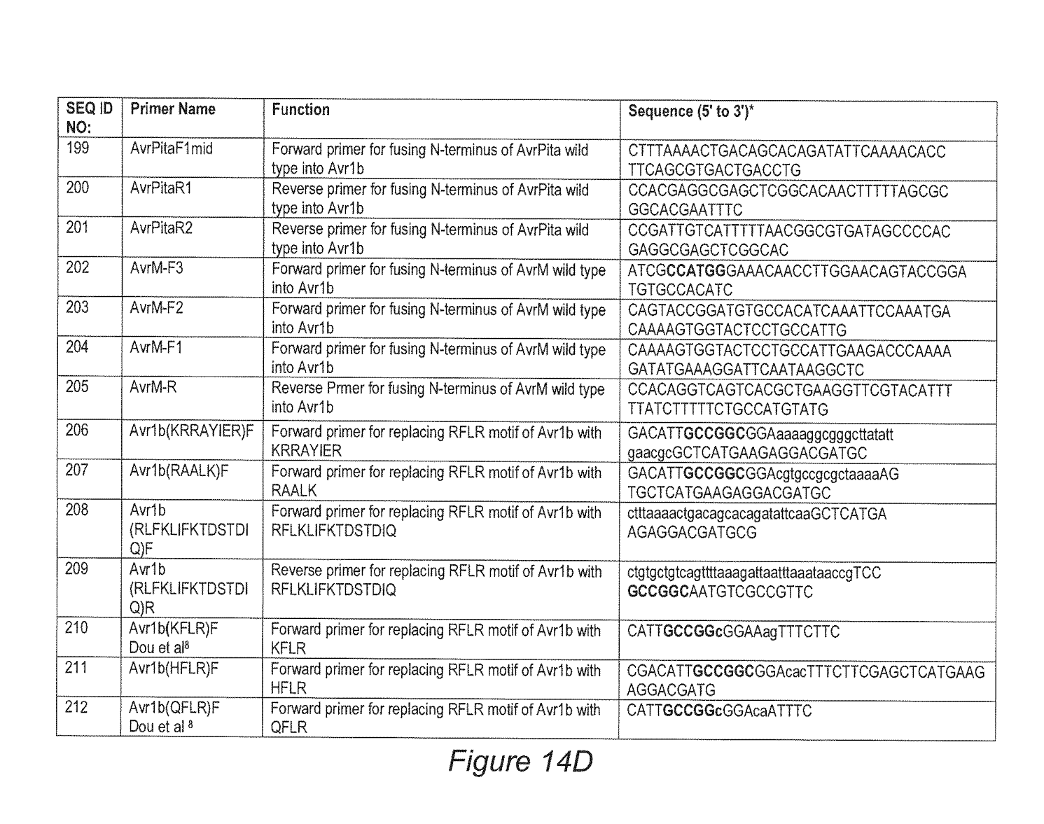

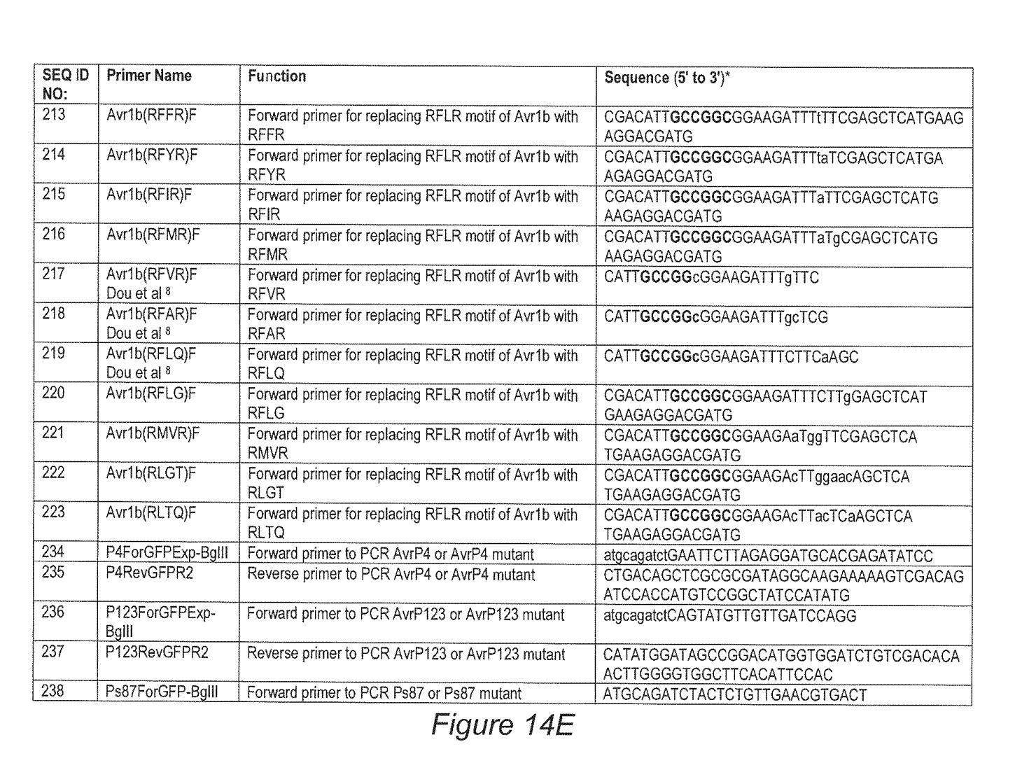

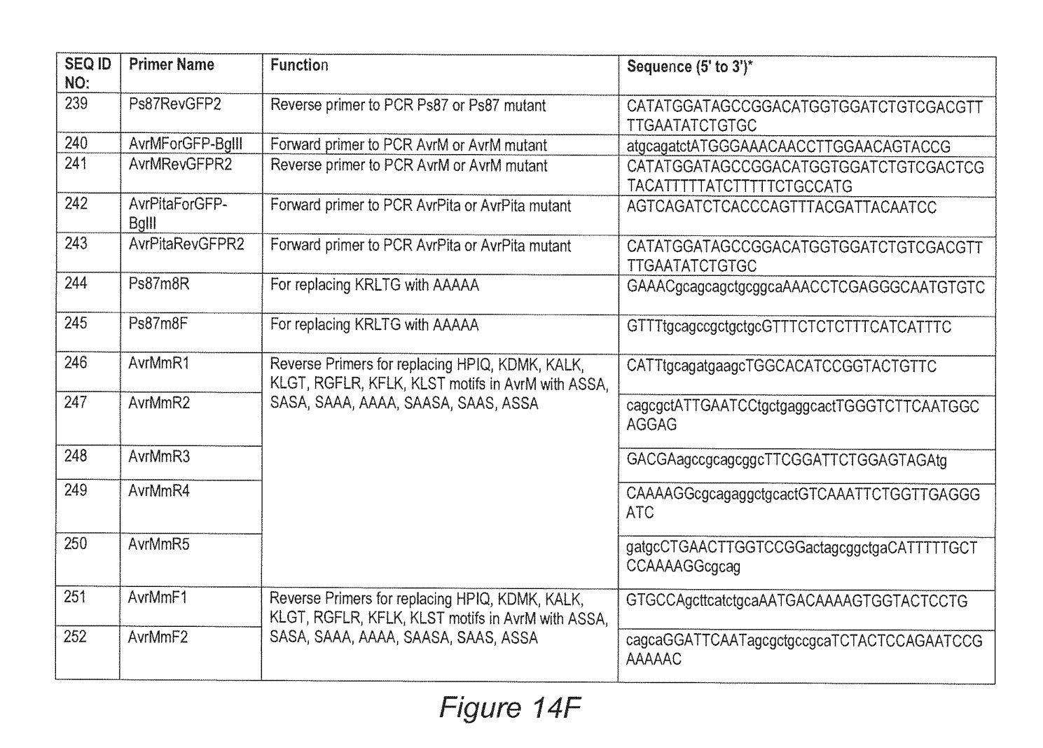

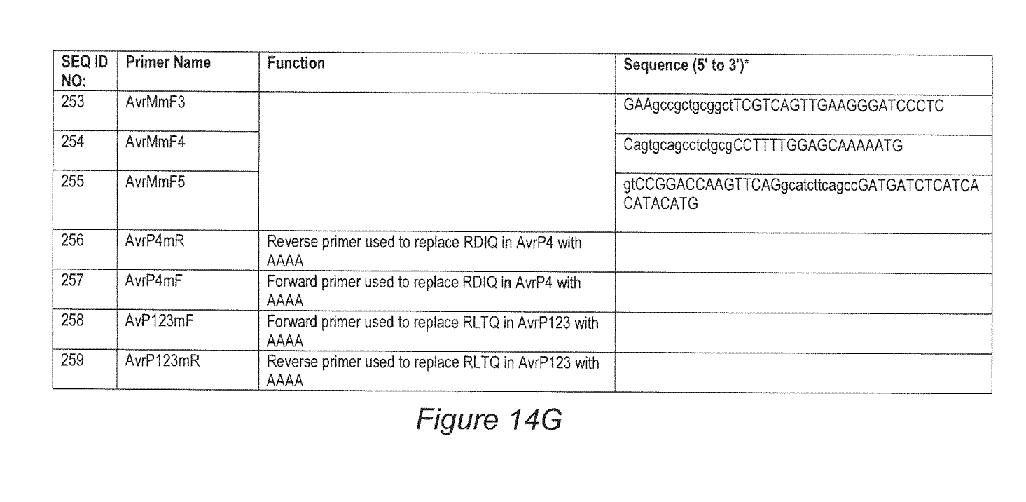

Cloning was performed according to known molecular biology techniques. Proteins were expressed in E. coli BL21DE3 and purified using affinity chromatography. Plasmids and oligonucleotides used in the study are depicted in tabular form as FIGS. 13A-F and FIGS. 14A-D, respectively.

Soybean seeds were germinated in vermiculite for 3-5 days. Roots were washed with water thoroughly to remove any debris. Approximately 1.5 cm root tips were cut and placed into the protein solution (50 μl-25 mM MES pH 5.8, 50 μg protein) and incubated for 12-15 hr at 28° C. Then the root tips were rinsed with water and washed in 75 mL of water for 2 hr on an orbital shaker at 90 rpm. Roots were examined using a Zeiss LSM510 laser scanning confocal microscope with an argon laser excitation wavelength of 488 nm.

Lipid filter arrays were prepared by pipetting 1 μL PI-3-P, PI-5-P (Cayman Chemical), PS, PC, PE, PA, or PI-4-P (Avanti Polar Lipids, Cayman Chemical) at various concentrations on Hybond-C extra membranes.

Liposomes were prepared from a suspension of 0.71 μg/ml phosphatidyl-choline, 0.29 μg/ml phosphatidyl-ethanolamine (PC/PE) or 0.64 μg/ml phosphatidyl-choline, 0.26 μg/ml phosphatidyl-ethanolamine, 0.1 μg/ml phosphatidyl-inositol-phosphate (PC/PE/PI-x-P). The lipid mixtures were dried under vacuum overnight, then the resultant lipid films were rehydrated at 1 mg/mL (total lipid) in 20 mM Tris-HCl (pH 6.8) 100 mM NaCl, 2 mM dithiothreitol by three cycles of freeze-thawing. Large unilamellar vesicles were formed by extruding the lipid suspension through a 0.1-μM filter (nucleopore track-etch membrane, Whatman) 20 times and were used immediately. Effector fusion proteins were centrifuged at 100,000 g for 20 min at 25° C. prior to assay to remove protein aggregates. 10 μg of protein was added to 50 μg of liposomes and incubated for 1 hr at room temperature. Protein-liposome mixtures were centrifuged at 100,000 g for 15 min at 25° C. Pellets containing liposome-bound proteins and supernatants containing free proteins, were then analyzed by SDS-PAGE.

### Example 3

### Assay for Screening Compound Libraries to Identify Novel Compounds that Interfere with the RXLR and dEER-Mediated Uptake of Effector Proteins into Plant or Human Cells

The binding of phosphoinositides (PI-3-P or PI-4-P) and phosphatidic acid to effector cell entry domains indicates that these phospholipids may serve as a cell entry receptors. Increasing the concentration of free phosphoinositide such as di-octanoyl-PI-4-P by exogenous addition stimulated RXLR and dEER-mediated uptake of the Avr1b GFP fusion, Avr1b(N)-GFP, into soybean roots and human cells. Furthermore, preincubation with inositol 1,4 diphosphate (IP2) inhibited binding of Avr1b(N)-GFP to PI-4-P-containing liposomes presumably via competitive inhibition. In addition, 1P2 almost completely blocked uptake of both Avr1b(N)-GFP into soybean root cells and human cells in cell culture. Therefore, an assay is devised for screening compound libraries to identify novel compounds that interfere with the RXLR and dEER-mediated uptake of effector proteins into plant or human cells, through inhibition of the binding of, or interaction between phospholipids PI-3-P or PI-4-P and RXLR and dEER motif containing proteins. Plasmids encoding the Avr1b sequence are expressed in BL21 E. coli cells and the protein are purified and diluted into appropriate binding buffer at an appropriate concentration, and thirty microliters are dispensed into each well coated 96 or 384 well plates using an automated dispenser. Through a robotized transfer mechanism involving steel pins, each of the Avr1b protein-containing wells (in a 96 or 384 well plate) receive 300 nanoliter of a compound from the compound libraries, followed by incubating the plate at room temperature for 60 minutes. An equal volume of 2× stock solution of fluorescently labeled soluble PI-4-P (Echelon Inc. BODIPY FL Phosphatidylinositol(4) Phosphate catalog #C-04F6a; BF-PI-4-P) is prepared in suitable buffer and 30 microliter of this solution is dispensed into each well of Avr1b coated preincubated 384 well plates using an automated dispenser. After BF-PI-4-P addition, the plate is incubated in dark for 60 minutes, followed by the measurement of fluorescence, utilizing a Synergy plate reader integrated with a biostack. The reactions are performed in duplicates and with negative controls, where the interactions are measured in the absence of the protein or fluorescently labeled BF-PI-4-P, and positive controls where the interaction is measured in the presence of a range of concentrations of inositol 1,4 diphosphate (IP2). The readouts are stored and analyzed for the identification of potential inhibitors of the reaction. Statistical analysis are performed utilizing a combination of parameters and compounds that showed statistically significant inhibition are selected. Briefly, the background absorbance is subtracted from the test reads. Subsequently, the net absorbance is compared to controls wells, that did not receive the test compounds and the percent decrease in absorbance is measured by the following formula: Percent inhibition=[(Fluorescence in test well/Fluorescence in control wells)×100]. In excess of one hundred thousand drug-like, diverse heterocyclic chemical compounds are screened during this process for their potential to inhibit the interactions between BF-PI-4-P and Avr1b. These compounds are obtained from several sources including established chemical vendors like Asinex, Analyticon, Biomol, Bionet, ChemDiv, Enamine, Maybridge, Spectrum, TimTec as well as a range of diversity oriented synthesis compounds that have been generated by academic research laboratories from around the world. Typical screening identifies several hundred compounds that inhibit the reaction at a statistically significant >40% levels. Successful events in this initial screen lead to the consolidation of select wells from the original library stock to generate a new second generation of plate for screening the activity of these compounds at three compound concentrations to allow the calculation of a preliminary 1050 value. A select group of compounds is then selected that showed >50% inhibition. Larger quantities of select compounds are ordered from the specific vendors (above) for rescreening in the soybean root or human cell uptake assays for their potential to inhibit the uptake of Avr1b(N)-GFP into soybean roots cells or human cells in culture.

### Example 4

### Screening Assays for Novel Compounds that Inhibit Plant Oomycete or Fungal Infection Through Blocking of RXLR and dEER Containing Effector Protein Action

To further characterize candidate protective compounds obtained from the RXLR and dEER protein screen, the ability of the compounds to protect against oomycete or fungal pathogen infection are tested in detached leaf assays. The following detached leaf assays are used for soybean, potato, tomato, tobacco, grape, rice, and wheat. The assays are used to test for infection by Phytophthora oomycete pathogens (soybean, potato, tomato, tobacco), downy mildew oomycete pathogens (tobacco and grape), rust fungi (soybean and wheat), Magnaporthe blast fungi (rice and wheat), and powdery mildew fungi (soybean, potato, tomato, tobacco, grape, wheat).

Expanded leaves are removed from young growth chamber-grown plants with the petioles intact (soybean, potato, tomato, tobacco, grape), or are clipped from the mother plant with sterile scissors (wheat and rice). The petioles or cut ends of the leaves are placed into plastic test tubes containing an aqueous solution of a suitable range of concentrations of each compound (determined from the biochemical IC₅₀). The leaves are then fastened into a horizontal position, but with the petioles or cut ends bent down into the tubes. The plants are then placed in a lighted growth chamber at 30% humidity for 6 hr to enable the compounds to be drawn into the leaves by transpiration.

The plants are then inoculated with pathogen spores. Phytophthora infections (P. sojae on soybean; P. infestans on tomato and potato; P. parasitica on tobacco) are initiated by spraying the leaves with an aqueous suspension of zoospores at a suitable concentration. Infections with rust fungi (Phakopsora pachyrizi for soybean; Puccinia striiformis f. sp. tritici (stripe rust) Puccinia triticina (leaf rust), Puccinia graminis f. sp. tritici (stem rust) for wheat) are initiated by spraying the leaves with an aqueous suspension of urediniospores at a suitable concentration. Infections with downy mildew oomycetes (Peronsopora tabacina on tobacco; Plasmopora viticola on grape) and infections of Magnaporthe blast fungi (Magnaporthe oryzae for rice; Magnaporthe grisea on wheat) are initiated by spraying the leaves with an aqueous suspension of conidia at a suitable concentration. In each case, after spraying with the pathogen spore suspension, the plants are replaced into the growth chamber at high (90%) humidity at a suitable temperature (15° C. for Phytophthora infestans and downy mildew oomycetes; 25° C. for P. sojae; 20° C. for all others) until symptoms develop (3-7 days). Infections with powdery mildew (Microsphaera diffusa on soybean; Erysiphe cichoracearum on potato and tobacco; Leveillula taurica on tomato or potato; Erysiphe necator on grape; Blumeria graminis f. sp. tritici on wheat) are done dry. The arrays of plant leaves are placed into a dusting tower and heavily infected leaves are introduced into the top of the tower and shaken vigorously for one minute, then the spores are allowed to settle for 20 min. The plants are replaced into growth chambers maintained at 70% humidity (except Microsphaera diffusa which is favored by low humidity of 30%) and 25° C.

To assay disease development in each case, the leaves are photographed, and from the photographs the numbers of lesions on each leaf are counted (total lesions and sporulating lesion) and the areas of the lesions are determined digitally. The results are assessed statistically by reference to negative controls (water), positive controls (benomyl for the fungi and metalaxyl for the oomycetes).

### Example 5

### Protecting Plants Against Oomycete Infection by Blocking Effector Entry Using Peptide Receptor Mimics

Eukaryotic pathogens such as oomycetes, fungi and apicomplexan parasites deliver hundreds of effector proteins into the cytoplasm of their host cells. Delivery of these proteins is key to the pathogenic success of these organisms. The similarity between oomycete and apicomplexan effector delivery systems has been noted for some time. The discovery that inositol 1,4 diphosphate can inhibit oomycete and fungal effector uptake (Example 2) shows that effector entry can be blocked by externally applied small molecular weight compounds. This Example describes experiments that test whether infection by oomycetes, and possibly by fungi, can be mitigated by inhibiting effector entry using host-synthesized peptides that mimic inositol 1,4 diphosphate. Biotrophic and hemi-biotrophic oomycete pathogens that are likely to use RXLR and dEER effectors include more than 80 species of Phytophthora and more than 500 species of downy mildews that together attack almost every crop species and horticultural species of economic importance. Peptides that could inhibit RXLR and dEER effector entry could thus provide broad-spectrum protection against many of these pathogens. Even if protection is narrow, and multiple peptides must be selected for each species of pathogen, this approach offers an important new weapon against these highly adaptable pathogens. The fact that one fungal effector from a rust pathogen also may use phosphoinositides to enter host cells suggests that the protection provided by anti-RXLR and dEER peptides may extend to many biotrophic and hemi-biotrophic fungal pathogens such as rusts, smuts, powdery mildews and the rice blast fungus.

At least three commercially available phage display libraries are screened against a panel of effectors that have been well-characterized and/or are strongly expressed at the outset of P. sojae or P. infestans infection. The phage are eluted from the effectors using a rising concentration gradient of IP2 or soluble PI-P in order to identify those phage that have the greatest affinity for the PI-P binding sites of the effectors. The candidates obtained are evaluated for their binding to all panel members, and to RXLR and dEER mutants of the panel members. In addition, their affinity for both soluble and liposome-bound PI-4-P and PI-3-P is measured. Synthetic peptides corresponding to candidates with the highest affinities are prepared commercially and tested for their ability to inhibit uptake into plant and human cells. The most promising peptides at this point (broadest specificity, highest affinity and/or strongest inhibition of effector entry) are targeted for optimization of their breadth and affinity of binding. Two optimization strategies are used. Firstly, PCR-directed random mutagenesis of selected peptides is carried out and high affinity, broad-spectrum mutants are selected by phage display. Loss-of-activity mutants are also characterized to help identify important residues. As a parallel alternative approach, targeted mutagenesis of selected peptides is carried out based on bioinformatic analysis of all the phage peptide sequences obtained (both high quality and low quality peptides). Surface Plasmon resonance and NMR characterization of the binding of the most promising peptides to their target effector(s) also yields important information. The cycle of selecting promising peptides and optimizing them is repeated as needed, or until little further improvement is obtained. At this point the most promising peptides are fused to selected secreted plant proteins, and the chimeric proteins are expressed in plant tissues by transient expression. The expression levels and stabilities of the chimeric proteins are assessed, as well as the ability of the expressed proteins to reduce entry of effector proteins and reduce infection by P. sojae or P. infestans. The resistance of the plant tissue to additional pathogens is also evaluated. Stable transgenic plants expressing the chimeric proteins are produced, and are evaluated systematically for disease resistance against diverse pathogens.

Initially the Ph.D.-C7C random peptide phage display library available from New England Biolabs, Inc. is screened. In this library, a loop of 7 random amino acid residues is constrained by a disulfide bond at the base of the loop where it is fused to the N-terminus of the pill coat protein. This configuration was chosen because the loop will eventually be transferred to a secreted plant protein, and the disulfide bond will ensure that the loop has a similar structure in that context as on the phage. The library contains 1.2×10⁹ independent phage, providing about 60% statistical coverage of the total theoretical complexity of a heptapeptide library (207=1.3×10⁹). Other possible libraries for screening include Ph.D.-7 and the Ph.D.-12 libraries that contain 7 or 12 random residues respectively, but without a disulfide bond; both have a complexity of around 2.8×10⁹.

Two approaches are used to select RXLR and dEER-specific phage. In the first, individual effectors are screened. Since this allows interactions with residues outside the immediate RXLR and dEER region, inhibitory peptides with a narrow specificity are likely to be obtained. In a complementary approach, the phage are selected on several different effectors successively, in order to only obtain peptides with broad specificity. Both strategies are adjusted as needed.

The one-effector-at-a-time strategy targets Avr1b, Avh331 (Avr1k), Avh5, Avh6 and Avh172. Because the first two effectors are avirulence gene products that trigger plant defense responses mediated by resistance (R) genes, the efficacy of candidate inhibitory peptides is also tested in planta by their ability to inhibit the R gene mediated response to the effectors. Avh6 and Avh172 are major early-expressed effectors, so targeting them singly also has a measurable effect on pathogen virulence. Avh5 is included because its NMR characterization is well advanced. In each case, two different fusions are produced: GFP and GST (glutathione-S-transferase) to reduce the chance of selecting phage that bind to an irrelevant part of the protein.

In the second strategy, three pools of effectors are created, and the phage are successively selected on the different pools. One example of such a set of pools is: pool 1=Avr1b+Avh331+Avh6; pool 2=Avh5+Avh172+Avh152; pool 3=Avh38+Avh260+AvrL567. Each effector listed is either an avirulence protein or a strongly-early-expressed P. sojae effector, or is otherwise well-characterized. By using pools, the risk that a single chosen effector may be problematic is reduced, and by using three different pools for the successive selection steps, the likelihood of finding broad specificity peptides is increased. The order of the pools used for selection is varied. The composition of the pools may be varied once data on the specificity of each effector for PI-3-P or PI-4-P is available, and/or if production of some chosen effector proteins in E. coli proves problematic.

Panning is carried out in microtiter tray wells; if sufficient enrichment of peptides is not seen in the wells, then the proteins are bound onto beads and the beads are used for panning. The phage are step eluted with different concentrations of inositol diphosphates (1P2) or soluble (e.g. di-hexanoyl) phosphatidyl phosphates. The choice of 1,3 IP2, 1,4 IP2, PI-3-P or PI-4-P, and the choice of concentrations is finalized once more precise data on the binding constant of the effectors for the phosphoinositides is available.

Characterization of Discovered Peptides for Binding to Multiple Effectors, and for Ability to Block Entry of Key Oomycete and Fungal Effectors into Plant Cells.

Each selected peptide is screened against a panel of all the effectors mentioned listed above, plus a selection of 10 P. infestans infection-induced effectors and several fungal effectors. RXLR and dEER region mutants are included to identify peptides that interact with those motifs. Phage with the broad specificity and a set of peptides with complementary sets of targets are identified. Screening is done in a western dot blot format in which effectors bound to a filter are probed with the phage and then with an anti-M13 antibody. Alternatively, the phage are panned against effectors arrayed in microtiter wells, and then detected by spotting onto an E. coli lawn with a replicator. The affinity of the phage for the effector is initially estimated by doing binding experiments in the presence of different concentrations of PI-Ps or IP2s. An oomycete effector protein microarray containing all 1440 effectors from P. sofae, P. infestans, P. ramorum and H. arabidopsidis is ideal for comprehensive screening of the most promising phage. The most promising peptides are tested for the ability to block effector-GFP entry into root cells. To obtain sufficient peptides for these experiments, synthetic peptides are ordered from a commercial supplier. A quantitative cell entry assay using luciferase and suspension culture cells may also be employed. Binding of the most promising peptides to key effectors is further characterized by NMR and surface plasmon resonance (SPR).

Concatenation and Mutagenesis of the Most Promising Peptides to Further Optimize Broad-Spectrum Binding and Ability to Block Effector Entry into Plant Cells.

Bioinformatic comparisons of peptide sequences having different affinities and ranges of specificity provide important starting clues about the potential for using targeted mutations to improve affinity and specificity of the identified peptides. Alternatively, phage display technology is a proven platform for improving binding via random mutagenesis. A single randomized oligonucleotide is used to mutagenize the 21 nucleotides encoding each peptide loop. Selection of phage on a range of different effectors is used to improve the breadth of specificity. Selection of phage in the presence of free peptide having the original sequence is used to select for improved affinity. An alternative approach to improving the breadth of specificity is to concatenate several peptides, with a spacer or linker sequence in between. This is an acceptable construction for in planta expression. The concatenated peptides are tested to ensure that they retain their original affinities and breadth of specificity.

Fuse Peptides to Small Secreted Plant Proteins, and Test the Effects of their Expression in Planta on Effector Entry and on Disease Resistance.

For evaluation in planta, the peptide mimics are fused to larger proteins normally produced during infection to promote the peptides' stability and reduce their potential susceptibility to endogenous plant proteases. The fusions are evaluated in three steps: (i) exogenous application of purified proteins to plant tissues; (ii) transient expression in plants; and (iii) expression in stable transgenic plants. A variety of candidate proteins are evaluated for fusions with the peptide mimics, including highly stable plant proteins such as PR1a, lipid transfer proteins, protease inhibitors and proteases. Fusion to a protease inhibitor promotes stability, while conversely, fusion to a protease more effectively targets pathogen effectors for proteolysis. Generally, the mimic is attached to the C-terminus of the “carrier” protein via a suitable spacer so that the native N-terminal secretory leader can be used. Initially a single peptide mimic is attached to each carrier. Once attachment of single peptides has been validated, multiple peptides are attached in tandem to improve the breadth of binding and/or for better efficacy against effectors with multiple phophoinositide binding sites. C-terminal green fluorescent protein (GFP) fusions are used to evaluate the stability and localization of the proteins in planta.

Expression in E. coli or Pichia pastoris and Evaluation of Purified Proteins.

Fusion proteins are expressed in E. coli or, due to the necessity to correctly form disulfide bonds, in eukaryotic expression system based on Pichia pastoris. The purified peptide-fusion proteins are tested for effector binding in vitro to ensure they retain binding activity as fusions. They are then introduced into leaf and root tissues (by infiltration and direct uptake, respectively) from soybean and N. benthamiana to test their stability in planta (via western blots) and to test their ability to inhibit the uptake of exogenously applied effector-reporter fusions into the plant cells. Uptake assays based on suspension cultures cells and on protoplasts may also be used to distinguish between stability and effectiveness in effector uptake inhibition.

### Transient Expression in Planta.

Excellent virus-based transient expression systems now exist for soybean and Arabidopsis. Infiltration of Agrobacterium tumefaciens strains harboring vectors designed to deliver gene expression constructs into plant cells locally (Agroinfiltration) and particle bombardment have also been used extensively. Initially a quantitative “double-barrel” particle bombardment assay is used to measure the ability of plant-expressed peptide fusion proteins to interfere with effector entry into soybean cells, using either the native effectors, Avr1b or Avh331 (i.e. Avr1k), or fusions of other effectors to an Avr1b reporter. By using GFP-fusions in conjunction with onion epidermal cell bombardment direct visualization of the localization of the peptide fusions or the targeted effector (or both together if one carries a red fluorescent protein, e.g mCherry) is possible. Bi-molecular fluorescence complementation (BiFC; “split-YFP”) is used to verify effector-peptide interaction in planta in the onion system.

In order to evaluate the potential effect of peptide-fusion expression in planta on pathogen infection, the BPMV system is used to transiently express the peptide fusion proteins in soybean and Agroinfiltration to transiently express the proteins in N. benthamiana. Versions that include GFP to facilitate evaluation of stability and localization are used. Transcription of the constructs is confirmed using RT-PCR or northern analysis. Protein levels are evaluated by western blots, and confocal microscopy is used to verify that the proteins are being delivered to the apoplast.

Areas of plant tissue transiently expressing the peptide fusions are inoculated with P. sojae (soybean) or P. infestans (N. benthamiana) and disease development is evaluated. Empty vectors and vectors with the entire construct minus the peptide mimic are used as negative controls in these experiments. N. benthamiana tissue is tested for its response to the blue mold downy mildew pathogen Peronospora tabacina. Soybean leaf tissue is tested for resistance to the soybean rust fungus, Phakopsora pachyrhizi.

Hairy root cultures of soybean expressing the peptides are created, and assayed them for P. sojae resistance, to show that expression of Avr1b and Avh331 confers increased susceptibility to P. sojae. Stably transformed soybean, N. benthamiana and Arabidopsis plants expressing the fusion proteins are tested and display resistance to a variety of oomycete and fungal pathogens.

### Example 6

### Identification of Additional Virulence Motifs

The previous Examples present results showing that a fungal effector protein, AvrL567, from a rust fungus that forms haustoria (specialized feeding structures) enters plant cells via RXLR sequence motif-mediated binding to a phosphatidylinositide within the plant cell wall. In this Example, results are presented which show that effector protein Avr2 from the tomato pathogen Fusarium oxysporum Esp. lycopersici and effector protein AvrLm6 from the Brassica pathogen Leptosphaeria maculans also enter plants via the same mechanism. Fusarium oxysporum f.sp. lycopersici is a xylem dwelling pathogen and Leptosphaeria maculans is an apoplastic pathogen.

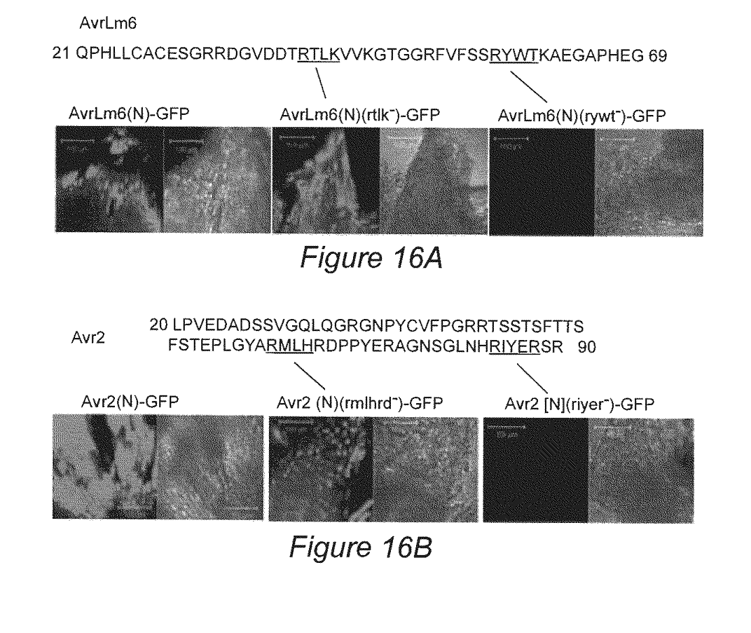

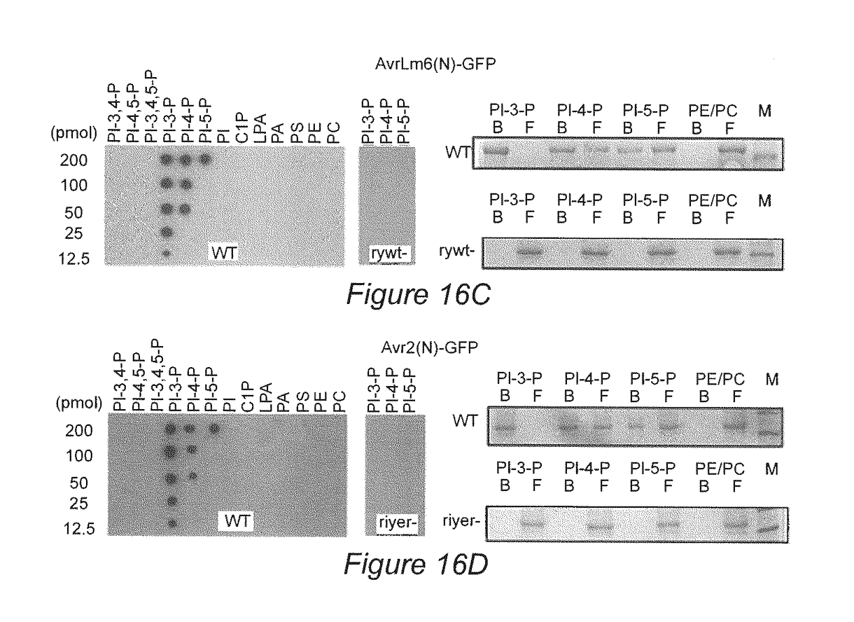

AvrLm6 contains two RXLR-like sequences, RYWT and RTLK. Mutations in the second motif (RYWT) but not the first (RTLK) abolish entry of AvrLm6-GFP fusions into root cells (FIG. 16A). Avr2 also contains two RXLR-like sequences, RMLH and RIYER. Mutations in the second motif (RIYER) but not the first (RMLH) abolish entry of Avr2-green fluorescent protein (GFP) fusion proteins into root cells (FIG. 16B). Both effector-GFP fusions bind PI-3-P strongly, PI-4-P moderately and PI-5-P weakly, but there was no binding to any other lipids (FIGS. 16C and D). In each case, the PI-P binding is dependent on the functional RXLR-like sequences, RYWT (in AvrLm6) and RIYER (in Avr2). Entry of AvrLm6 (FIG. 16E) and Avr2 (FIG. 16F) is inhibited in both cases by inositol 1,4 diphosphate (FIG. 16E), inositol 1,3 diphosphate (not shown) and by the PI-3-P binding proteins VAMp7 PX, indicating that entry into the soybean root cells is dependent on the presence of PI-3-P.

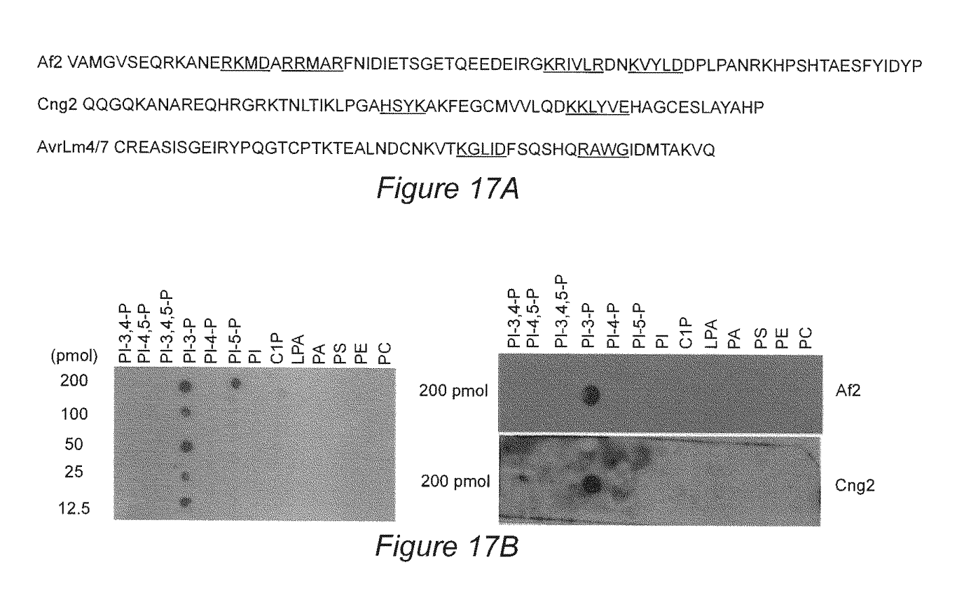

It has been found that GFP fusions to the N-termini of three more fungal effectors or effector-like proteins bind PI-3-P. The proteins are Leptosphaeria maculans effector AvrLm4/7 and two bioinformatically-predicted effector-like proteins from the human pathogens Cryptococcus neoformans (Cng2; AAW43853.1) and Aspergillus fumigatus (Af2; XP_{—}752996.1). Each N-terminal domain contains potential RXLR-like motifs (FIG. 17A).

These findings extend previously findings that several effectors from oomycete plant pathogens and apicomplexan pathogens of vertebrates (e.g. Plasmodium falciparum) bind phosphoinositides, particularly PI-3-P, which enables them to enter plant and animal cells.

### Example 7

### Occurrence of RXLR-Like Motifs in Effector Like Proteins from a Wide Diversity of Oomycetes, Fungi and Insects

Using a bioinformatic approach informed by detailed mutagenesis of the Avr1b RXLR motif, we have identified candidate RXLR-like motifs in 20 experimentally validated fungal effectors, as well as in 13 experimentally validated oomycete effectors (Table 1). The fungal effectors include an effector (MiSSP7) from a mutualistic ectomycorrhizal fungus, Laccaria bicolor (Martin et al., 2008).

Some sucking and chewing insects produce effector-like proteins, including hessian flies (Mayetiola destructor) (Behura et al., 2004) and pea aphids (Acyrthosiphon pisum) (Mutti et al., 2008). We have identified candidate RXLR-like motifs in the N-terminus of effectors vH9 and vH13 from Mayetiola destructor, and in the effector C002 from Acyrthosiphon pisum (Table 3).

Among oomycetes, RXLR-containing effectors have so far been documented in pathogens from the order Peronosporales. We have identified RXLR-like motifs in bioinformatically predicted effectors from Pythium ultimum (Pythiales) and Albugo candida (Albuginales), suggesting that RXLR-like effectors may be common to the entire oomycete Phylum (Table 4).

We have also identified RXLR-like motifs in bioinformatically predicted effectors from the necrotrophic plant pathogens Pyrenophora tritici-repentis and Alternaria brassicicola, and from the human pathogens Cryptococcus neoformans, Aspergillus fumigatus and Coccidioides immitus (Table 4).

Thus, phosphoinositide binding, particularly to PI-3-P, is a common property of most if not all eukaryotic effectors that can autonomously enter host plant or animal cells across their plasma membranes, including effectors produced by host-associated oomycetes, fungi and animals (e.g. insects and nematodes), and including pathogens, mutualists, commensals, ectosymbionts and endosymbionts. A further corollary is that chemical or transgenic control measures that target the interaction of RXLR-like sequences with phosphoinositides will potentially be effective against a wide range of oomycete, fungal and animal pathogens.

TABLE 3
RXLR-like sequences in experimentally verified effectors.

| | | King- | N-terminal |
|---|---|---|---|
| Effector | Species | dom | Amino Acid Sequence |
| Avr1a | Phytophthora | O | SENAFSAATDADQATVSKLAAAEFDTLVDV |
| | sojae | | LTTESKRSLRATVDDGEER |
| | | | (SEQ ID NO: 304) |
| Avr1b | Phytophthora | O | TEYSDETNIAMVESPDLVRRSLRNGDIAGGR |
| | sojae | | FLRAHEEDDAGERTFSV (SEQ ID NO: 305) |
| Avr1k | Phytophthora | O | LTCATSEQQTRPELCFFFSVRSSWPSTISDGA |
| | sojae | | CLALVSAEQGATAGRNTLSLRSMMATEDM |
| | | | ATSTRSLRSQATNVDDDANVSIENR |
| | | | (SEQ ID NO: 306) |
| Avr3a | Phytophthora | O | LSTTNANQAKIIKGTSPGGHSPRLLRAYQPD |
| | sojae | | DEGDSPEDR (SEQ ID NO: 307) |
| Avr3c | Phytophthora | O | VEPSATSTVEVAEVQARGADKRFLRSLQTE |
| | sojae | | EE QGDSDVNEAEDGSEER |
| | | | (SEQ ID NO: 308) |
| Avr46 | Phytophthora | O | ITDESQPRDATIVDAPLTGRGANARYLRTST |
| | sojae | | SIIKAPDAQLPSTKAAIAS (SEQ ID NO: 309) |
| Avh172 | Phytophthora | O | TAEVDSKTALAAEVPAAIRSLESDTPASRLL |
| | sojae | | RTGTVTSADNEDR (SEQ ID NO: 310) |
| Avr3a | Phytophthora | O | IDQTKVLVYGTPAHYIHDSAGRRLLRKNEE |
| | infestans | | NEETSEER (SEQ ID NO: 311) |
| Avr4 | Phytophthora | O | KADSLARTVSVVDNVKVKSRFLRAQTDEK |
| | infestans | | NEER (SEQ ID NO: 312) |
| AvrBlb1 | Phytophthora | O | AVSSNLNTAVNYASTSKIRFLSTEYNADEKR |
| | infestans | | SLRGDYNNEVTKEPNTSDE |
| | | | (SEQ ID NO: 313) |
| AvrB1b2 | Phytophthora | O | VAAFPIPDESRPLSKTSPDTVAPRSLRIEAQE |
| | infestans | | VIQSGR (SEQ ID NO: 314) |
| Atr1 | Hyaloperonospora | O | TESSETSGTIVHVFPLRDVADHRNDA |
| | arabidopsidis | | LINRALRAQTALDDDEER (SEQ ID NO: 315) |
| Atr13 | Hyaloperonospora | O | LLHAHALHEDETGVTAGRQLRAAASEVFGL |
| | arabidopsidis | | SRASFGLGKAQDPLDKFF (SEQ ID NO: 316) |
| AvrLm1 | Leptosphaeria | F | SPATKNNVNQPLDNISRRSEWKSVQIS |
| | maculans | | PVKEHSAKTADNTENNHNLEKRVFTSP |
| | | | HMKRTFTLALENTFYAMAWLIDFSFS |
| | | | EEGEPHFSYKLQ (SEQ ID NO: 317) |
| AvrLm4/7 | Leptosphaeria | F | CREASISGEIRYPQGTCPTKTEALNDC |
| | maculans | | NKVTKGLIDFSQSHQRAWGIDMT |
| | | | (SEQ ID NO: 318) |
| AvrLm6 | Leptosphaeria | F | QPHLLCACESGRRDGVDDTRTLKVVKGTGG |
| | maculans | | RFVFSSRYWTKAEGAPHE (SEQ ID NO: 319) |
| Avr-Pita | Magnaporthe | F | HPVYDYNPIPNHIHGDLKRRAYIERYSQCS |
| | oryzae | | DSQASEIRAALKSCAELASWGYHAVKSD |
| | | | NRLFKLIFKTDSTDIQN (SEQ ID NO: 320) |
| Avr-Pii | Magnaporthe | F | LPTPASLNGNTEVATISDVKLEARSDTTYHK |
| | oryzae | | CSKCGYGSDDSDAYFNHKC |
| | | | (SEQ ID NO: 321) |
| Avr-Pia | Magnaporthe | F | RFCVYYDGHLPATRVLLMYVRIGTTATITA |
| | oryzae | | RGHEFEVEAKDQNCKVILTNG |
| | | | (SEQ ID NO: 322) |
| Avr-Pizt | Magnaporthe | F | SFVQCNHHLLYNGRHWGTIRKKAGWAV |
| | oryzae | | RFYEEKPGQPKRLVAICKNA |
| | | | (SEQ ID NO: 323) |
| Avr-PikD | Magnaporthe | F | ETGNKYIEKRAIDLSRERDPNFFDHPGIPVPE |
| | oryzae | | CFWFMFKNNVRQ (SEQ ID NO: 324) |
| Avr1 | Fusarium | F | LPKGEEGDIIGTFNFSSSDSQPLKIHWVDTPD |
| (Six4) | oxysporum f. sp. | | SSGSNLVKRSA (SEQ ID NO: 325) |
| | lycopersici | | |
| Avr2 | Fusarium | F | LPVEDADSSVGQLQGRGNPYCVFPGRRTSS |
| (Six3) | oxysporum f. sp. | | TSFTTSFSTEPLGYARMLHRDPPYERAGNSG |
| | lycopersici | | LNHRIYERSRVGGLRTVIDV |
| | | | (SEQ ID NO: 326) |
| Avr3 | Fusariuin | F | QEAAVREPQIFFNLTYTEYLDKVAASHGSPP |
| (Six1) | oxysporum f. sp. | | DKSDLPWNDTMGSFPGNETDDGVQTETGSS |
| | lycopersici | | LSRRGHIVNLRKREPFGEESRNDRVTQD |
| | | | (SEQ ID NO: 327) |
| Six2 | Fusarium | F | NPAGDSLPDDAHLPDRRLSPSEVQALKKAQ |
| | oxysporum f. sp. | | IYPPGYIHKRVTFGEGKDAV |
| | lycopersici | | (SEQ ID NO: 328) |
| Six5 | Fusarium | F | RDHQYCACQSGSGDSIDIDATTQLQNDNS |
| | oxysporum f. sp. | | KSYLWAQTSPAYWFADRHK |
| | lycopersici | | (SEQ ID NO: 329) |
| Six6 | Fusarium | F | GPLAQTESESADVAEHTINYIDIAPEEFEPPK |
| | oxysporum f. sp. | | ANLSSLVSRDTLPVST (SEQ ID NO: 330) |
| | lycopersici | | |
| AvrL567 | Melampsora lini | F | MEHVPAELTRVSEGYTRFYRSPTASVILSG |
| | | | LVKVKWDNEQMTMPLFKWIG |
| | | | (SEQ ID NO: 331) |
| AvrM | Melampsora lini | F | SLSNNLGTVPDVPHQIPNDKSGTPAIEDPKA |
| | | | AIEDPKDMKGFNKALKSTPESEKLGTSSVE |
| | | | GIPQPEFDRGFLRPFGAKMKFLKPDQVQ |
| | | | KLSTDDLITYM (SEQ ID NO: 332) |
| AvrP123 | Melampsora lini | F | QYVVDPGFGEIECMCGQIARLTQRPFDVE |
| | | | CEAT (SEQ ID NO: 333) |
| AvrP4 | Melampsora lini | F | EFLEDARDIOGFSRKSGSKLEEESDSSRDRQ |
| | | | (SEQ ID NO: 334) |
| STP1 | Ustilago maydis | F | NGSISNASHHHQRRMVRQRHIEARSAMSWL |
| | | | TKISSKASDWMFGSVHAPNLDKKDLPKPLV |
| | | | GGVAVMPKMPY (SEQ ID NO: 335) |
| MiSSP7 | Laccaria bicolor | F | SPVPGEVGLVERGPIPNAVFRRVPEPNFFKD |
| | | | LLRALGQASQGGDLHR (SEQ ID NO: 336) |
| C007 | Acyrthosiphon | I | SAAEPYDEQEEASVELPMEHRQCDEYKSKI |
| | pisum | | WDKAFSNQEAMQLMELTFNTGKELGSHEV |
| | | | (SEQ ID NO: 337) |
| vH13 | Mayetiola | I | SPLPLAYTDQVYDACDRQFDETVRNSQPL |
| | destructor | | (SEQ ID NO: 338) |
| v19 | Mayetiola | I | LVLDTRAMPETDFEKALKEWNRVQTLVLIA |
| | destructor | | PEQRRTMVLIAEHLTNLKKMNVDSPGGSFL |
| | | | YLKDGDPVIKLPSVEHFEITFRGPYGVDKNF |
| | | | SFYMPKLKKLIVRDADANDKKIIKFVSQHS |
| | | | RTLKTLDLVAANYRTLRTLGAMKHIEEFVT |
| | | | SPP (SEQ ID NO: 339) |

Effectors named AvrXXX are avirulence gene products.
Motifs experimentally verified as functional are in bold and underlined.
Motifs experimentally determined to be non-functional are marked in italics.
Putative motifs are underlined.
Sequences shown are from the N-terminus of the respective proteins.
Note that Laccaria bicolor is a mutulasitic fungus.
Kingdoms:
O = oomycetes;
F = fungi;
I = insects

TABLE 4
RXLR-like sequences in bioinformatically predicted effectors.

| Predicted | | King- | |
|---|---|---|---|
| Effector | Species | dom | N-terminal Amino Acid Sequence |
| Avh5 | Phytophthora | O | TRVPDDANLQSVNAPVQTVTRSRRFLRTADT |
| | sojae | | DIVYEPKVHNPGKKQVFIE (SEQ ID NO: 340) |
| PYU1_T005878 | Pythium | O | MMPSTDAHGYIAFPPAQYKDPATATNYNAIIT |
| | ultimum | | ASINTAFACKKWDDNPTANTKTF TAAFKKSG |
| | | | YTSLKQMLDKKVPGCQNSRTDFTPIKTKTYK |
| | | | TMEWQND (SEQ ID NO: 341) |
| PYU1_T001475 | Pythiun | O | HSQMTVPNPKFSDVSKANSPLGTIDGPTVMPP |
| | ultimum | | PAGQSYAMGTDTNIKAYVEAFAKQTKWKTL |
| | | | KDLIMDKYVEDGNIPDRACGLTDKTYMQPLP |
| | | | DKYVVW (SEQ ID NO: 342) |
| AcEff1 | Albugo | O | STMGLSNSRHLEDAVERVLGDLKLNQKDEKQ |
| | candida | | NENEVDKNNSKGKDRESS (SEQ ID NO: 343) |
| Af13 | Aspergillus | F | AGLPVFPNQAVLRPSLALPGDNSHRYSLPMFD |
| | flavus | | LQPWERVDEIRLARKGYLYGSP |
| | | | (SEQ ID NO: 344) |
| PTRG_02320 | Pyrenophora | F | QVKGNAIRCGQDDKSDQDTRNFCRFMFTSD |
| | triticirepentis | | RTLKINGEFRGNA (SEQ ID NO: 345) |
| AB09791.1 | Alternaria | F | APASYPASALGKRWVDTTGGQKMPAHFVST |
| | brassicicola | | VRKLSTEKLKTKRQLDQLL (SEQ ID NO: 346) |
| Cng1 | Cryptococcus | F | LTVPQAHRETLEEAGKLTTIAAINTKKLIKDV |
| | neoformans | | TVGTMSSVFPDGTDNGGRP (SEQ ID NO: 347) |
| Cng2 | Cryptococcus | F | IQQGQKANAREQHRGRKTNLTIKLPGAHSYK |
| | neoformans | | AKFEGCMVVLQDKKLYVEHAGCESLAYAHP |
| | | | (SEQ ID NO: 348) |
| Af1 | Aspergillus | F | IPSAFPQDNAVNQVLLSDSYQDQSVSSISAED |
| | fumigatus | | DAQNSAVIHIGESETMRAPSWFTSTLMARRLL |
| | | | ALSTTGTVSTIFPDPLPGNSHAPPSVAGLP |
| | | | (SEQ ID NO: 349) |
| Af2 | Aspergillus | F | VAMGVSEQRKANERKMDARRMARFNIDIETS |
| | fumigatus | | GETQEEDEIRGKRIVLRDNKVYLDDPLPANRK |
| | | | HPSHTAESFYIDYP (SEQ ID NO: 350) |
| Af3 | Aspergillus | F | PVVPGQTVMEPSAALPDDGDHLYTLPMFDIR |
| | fumigatus | | PWERVSEVRLAREGYLYG (SEQ ID NO: 351) |
| Ci1 | Coccidioides | F | SPVFPGGDKRDALYQKPIAPAGEFPFDSSPPEA |
| | immitus | | RMTIPYADNEPDSSLSIPSWPTTHLLARRLLGL |
| | | | STTGVLSTVFPRTNRDPALVGVP |
| | | | (SEQ ID NO: 352) |
| Ci2 | Coccidioides | F | IRSSQRSQRRQEHRSRKMNLIVSCSDPSRKSK |
| | immitus | | DVDGCFVVLRNHKLWIASRPSDGEANEPSDD |
| | | | ATRFKASLHQCHH (SEQ ID NO: 353) |

Motifs experimentally verified as functional are in bold and underlined.
Putative motifs are underlined.
Sequences shown are from the N-terminus of the respective proteins.
Note that Cryptococcus neoformans, Aspergillus fumigatus and Coccidioides immitus are human pathogens. Pyrenophora tritici-repentis and Alternaria brassicicola are necrotrophic plant pathogens.
Kingdoms:
O = oomycetes;
F = fungi.

### Example 8

### Phosphatidylinositol-3-phosphate is the Natural Target of RXLR(-Like) Effectors

Of the eight oomycete and fungal effectors tested to date, seven have a preference for binding to PI-3-P, and one (Avr1b) has a preference for PI-4-P. Neither of PI-3-P nor PI-4-P has been reported to occur in the outer leaflet of the plasma membrane of plant or animal cells (Boon et al., 2002). Two papers have reported secretion of PI-4-P by plant cells (Regente et al., 2008; Gonorazsky et al., 2008).

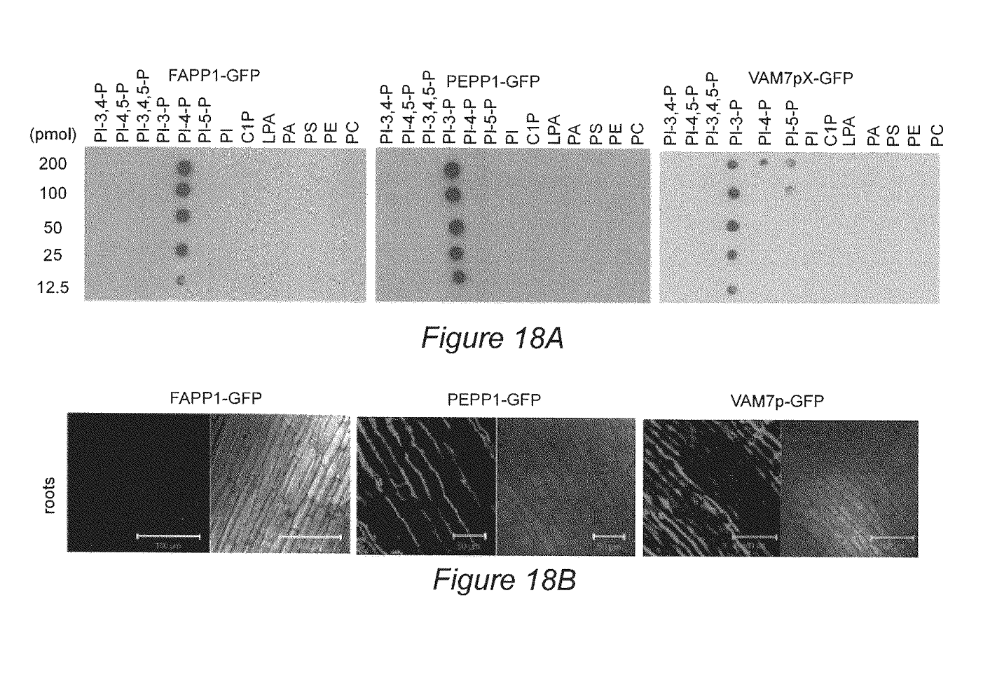

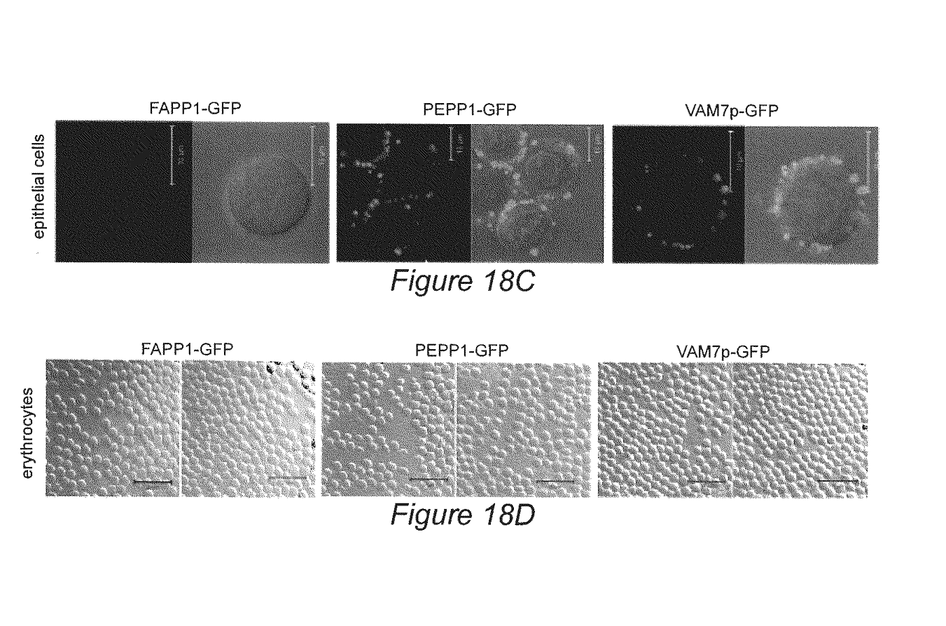

In order to test directly for the presence of PI-3-P and PI-4-P, we fused the PX domain of VAM7p and the PH domains of the human proteins FAPP1 and PEPP1 to green fluorescent protein (GFP). PEPP1 and FAPP1 bind very specifically to PI-3-P and PI-4-P, respectively (FIG. 18A) (Dowler et al., 2000). VAM7p has a preference for PI-3-P but can bind weakly to PI-4-P and PI-5-P (FIG. 18A) (Lee et al., 2006). The GFP fusion proteins were used to stain the surface of cells of human lung epithelial cell line A549 and soybean root cells. The results reveal very clearly that PI-3-P is present uniformly on the outer surface of roots cells (FIG. 18B), and at specific sites on the surface of the epithelial cells (FIG. 18C).

As can be seen, PI-4-P could not be detected in either case. Neither PI-3-P nor PI-4-P could be detected on the surface of erythrocytes. The lack of PI-3-P or PI-4-P on the surface of erythrocytes is significant because nearly all published studies documenting the absence of PI-3-P or PI-4-P from the outside of eukaryotic cells utilized erythrocytes. Our results suggest that erythrocytes may be an exception in this regard.

The finding of PI-3-P on the outside of plant and animal cells, combined with the preference for most effectors for PI-3-P is consistent with PI-3-P being the principal receptor mediating entry of the effectors into plant and animals cells. The absence of PI-4-P from the membranes suggests PI-4-P is unlikely to be the principal route of cell entry. However, PI-4-P has been reported to be secreted from plant cells under certain conditions (Regente et al., 2008; Gonorazky et al., 2008), and it is possible that some effectors such as Avr1b have evolved to respond to PI-4-P.

### Example 9

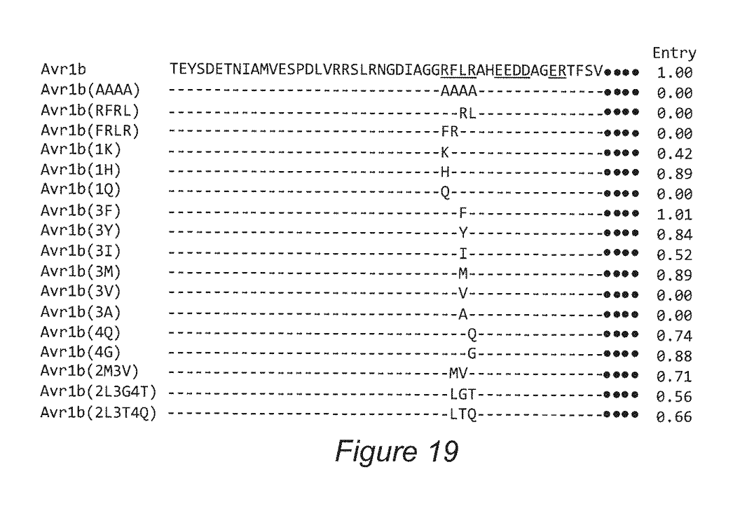

Since several of the fungal effectors that were tested contained no obvious RXLR or dEER motifs, we used the leaf bombardment assay to define the range of residues within the RXLR motif of Avr1b that could permit cell entry. The results (FIG. 19) revealed that lysine or histidine but not glutamine could replace the arginine at position 1 in the motif, that any large hydrophobic residue (isoleucine, methionine, phenylalanine, or tyrosine) could replace the leucine at position 3, but valine and alanine could not. At position 4, all residues tested allowed function. Furthermore, the presence of either a leucine or methionine residue at position 2 could substitute for leucine at position 3. The effector binding motif was thus refined to BXZ, where B=R, K or H; X is any amino acid and may be absent; and z=L, M, I, W, Y, or F.

### Example 10

### A PI-3-P-Binding Protein Blocks Entry of Effectors into Plant and Human Cells

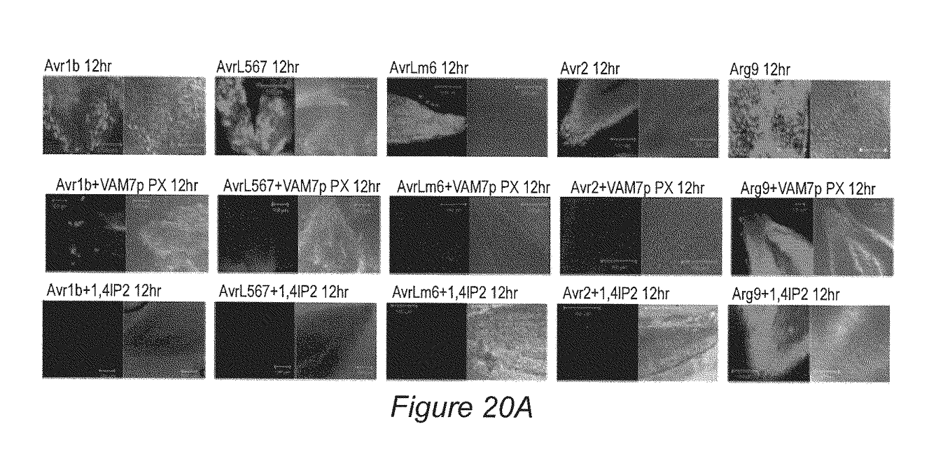

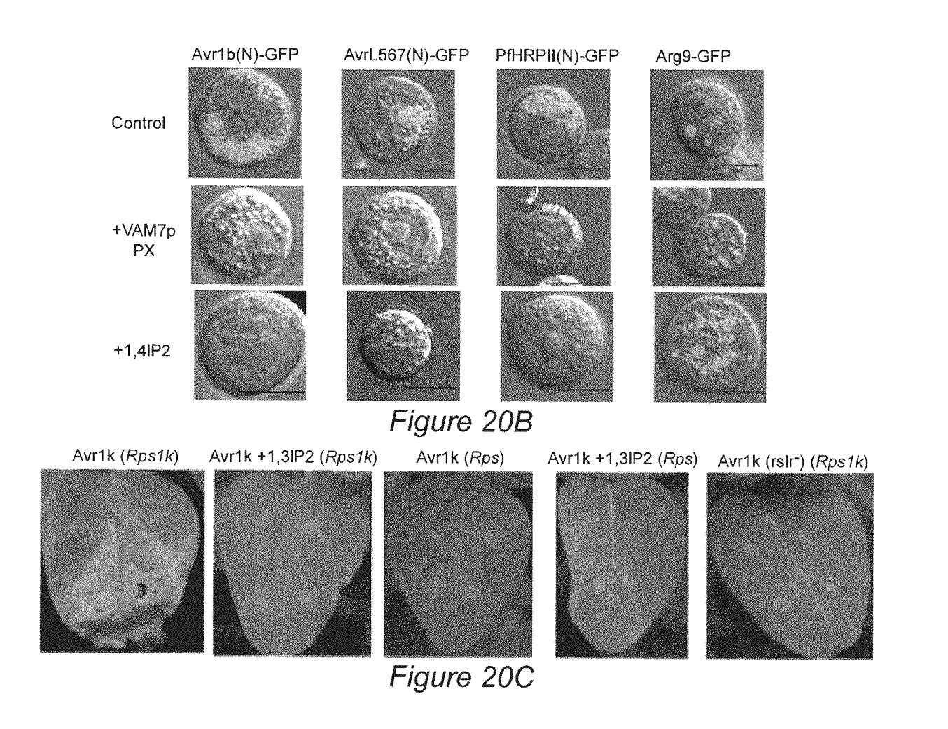

To determine more directly if PI-3-P mediates host cell entry, we pre-incubated soybean roots and epithelial cells with unlabeled VAM7p PX protein, which binds PI-3-P, prior to exposing the cells to effector-GFP fusions. Pre-incubation with VAM7p PX protein strongly inhibited entry by GFP fusions of the oomycete effector Avr1b, and the fungal effectors AvrL567, Avr2 and AvrLm6 into soybean root cells (FIG. 20A) but did not inhibit entry of a synthetic cell permeable protein Arg9-GFP that does not bind phosphoinositides. Similarly, VAM7p PX protein strongly inhibited the entry by GFP fusions of Avr1b, AvrL567 and the Plasmodium effector PfHRPII into human epithelial cells, but did not inhibit entry of Arg9-GFP.

These results strongly support the hypothesis that PI-3-P binding is necessary for the effector GFP fusions to enter plant and animal cells.

The previous examples showed that the head group mimic 1,4 inositol diphosphate (1,4IP2) could inhibit entry of effector-GFP fusions into soybean root cells and human epithelial cells (FIGS. 20A and B). Presumably the binding of 1,4IP2 to the effectors is strong enough to compete with binding for cellular PI-3-P.

To test the ability of inositol diphosphate to inhibit entry of a native effector (not a GFP fusion) into plant cells, we produced full length protein of the oomycete effector Avr1k (from Phytophthora sojae) in E. coli. We then infiltrated the purified protein into soybean leaves which did or did not carry the resistance gene Rps1k. In the presence of Rps1k, Avr1k triggers a programmed cell death response called the hypersensitive response (HR) (FIG. 20C, panel 1). In the presence of 1,3IP2, however, no HR was observed, consistent with 1,3IP2 blocking the entry of the effector into the leaf cells. When the RXLR motif of Avr1k (RSLR) was mutated, no HR was observed, even in the absence of 1,3IP2, confirming the RXLR motif was essential for cell entry. When the Rps1k gene was absent, no HR was observed, as expected. These results show that entry of RXLR effectors into plant cells requires binding to PI-3-P.

### Example 11

### Methods to Block Effector Entry Using Small-Molecule Drugs

The ability to block effector entry using 1,3IP2 or 1,4IP2 provides a proof-of-concept for treating oomycete or fungal infections of plants or animals, including humans, with drugs that block the PI-3-P-binding sites of the effectors. Such drugs may not need to fully block all effectors in order to be effective. Since a principal function of effectors is to suppress the host defense responses, even partial inhibition of effector entry may be sufficient to obtain protein. This point may be important because some forms of genetic resistance in plants (major gene resistance) rely upon entry of effectors into the plant cells (the resistance gene product encodes a receptor that detects the presence of an intracellular effector).

Drugs are also used to interfere with biosynthesis or export of PI-3-P to the outer leaflet. Inside the cell, PI-3-P can be formed by the action of phosphatidylinositol-3-kinases on phosphatidylinositol, and by the action of phosphatidylinositol 4,5 phosphatases on phosphatidylinositol 3,4 diphosphate, phosphatidylinositol 3,5 diphosphate and phosphatidylinositol 3,4,5 diphosphate. Any drug which inhibited these enzymes could lower the levels of external PI-3-P. Currently it is not known how PI-3-P reaches the outer leaflet. All known PI-3-P forming enzymes are located on the cytoplasmic face of membranes. PI-3-P could reach the external leaflet of the plasma membrane or the luminal face of secretory vesicles by the action of floppases or a scramblases. Alternatively PI-3-P might be transported to the outer leaflet by an ABC transporter or by a secreted lipid transfer protein. Any of the proteins involved in this process could be targeted with drugs, provided that they did not disrupt normal cell physiology. In addition to drugs that directly target the proteins described above, and drug that targets biosynthesis of the proteins, for example siRNAs, are also effective.

Some specific examples of drugs that may be used in the practice of the invention include but are not limited to membrane-permeant derivatives of inositol diphosphates (Li et al., 1992) and bis(hydroxymethyl)-inositol (Flu et al., 2000).

Alternatively, drugs that bind directly to PI-3-P making it unavailable to effectors are effective. For example, neomycin binds PI-4,5-P2 very effectively; thus neomycin, neomycin derivatives or other aminoglycosides that bind PI-3-P may be used.

If effective drugs cause toxicity, forms of the drugs which are activated only when in close proximity to the pathogen are used. For example, infection in both plant and animal cell systems results in local high concentrations of hydrogen peroxide. A pro-drug that is activated by oxidation or peroxidation mitigates toxicity.

### Example 12

### Methods to Block Effector Entry Using Polypeptides

Polypeptides with the properties described in Example 10 may also be utilized. Polypeptides may have some advantage over chemicals, in plant and animal systems, in that the host organism can be genetically engineered to produce the polypeptide, simplifying delivery and reducing cost. The ability to produce and select large numbers of variant polypeptides via phage display technologies provides additional power to improve specificity, if needed. Random peptides or single chain antibodies selected by phage display are used to block the PI-3-P binding sites of effectors. Additionally such effector-binding proteins could be fused to proteases to facilitate degradation of the effectors.

The ability to block effector entry by pre-incubation with PI-3-P-binding proteins provides a strong indication that secretion of PI-3-P-binding proteins could provide protection against infection, especially if the secreted protein could be targeted to the infection site. Additionally, secretion of enzymes which can hydrolyze PI-3-P or modify it in other ways may be effective in reducing the level of PI-3-P available to transport effectors into cells. Examples of such enzymes include but are not limited to PI-3-P 4,5 kinases, PI-3-phosphatases, or phospholipases, etc. Examples of these enzymes have been described in the literature (Falasca et al., 2006).

Additionally, enzymes (e.g. from microbes) that cause novel modifications of PI-3-P such as methylases, acetylases or glycosylases may be used. A particularly useful enzyme is a phospholipase C that can cleave PI-3-P into diacylglycerol and 1,3-inositol diphosphate; not only is the level of PI-3-P reduced but the inhibitor 1,3-inositol diphosphate would be produced as a result. Currently known phosphatidylinositol-specific phospholipase C's are specific for phosphatidylinositol, glycosyl phosphatidylinositol-protein anchors, or for phosphatidylinositol-4-phosphate and phosphatidylinositol-4,5-diphosphate. In some embodiments, systematic mutagenesis is used to modify the specificity of a phosphatidylinositol specific phospholipase C so that it could cleave PI-3-P.

Further, in cases where polypeptides that manipulate PI-3-P levels cause deleterious physiological effects on the host, transgenic hosts are produced in which the polypeptide gene is transcribed only during infection. Alternatively, or jointly with this strategy, the polypeptide is targeted to the site of infection. For example, the Arabidopsis protein. RPW8 is specifically targeted to haustoria of certain oomycetes and fungi (Wang et al., 2009). RPW8 is used to target anti-effector polypeptides to the haustorial space.

### REFERENCES FOR EXAMPLES 6-12

Behura, S. K., Valicente, F. H., Rider, S. D., Jr., Shun-Chen, M., Jackson, S., and Stuart, J. J. (2004). A physically anchored genetic map and linkage to avirulence reveals recombination suppression over the proximal region of Hessian fly chromosome A2. Genetics 167, 343-355.
Boon, J. M., and Smith, B. D. (2002). Chemical control of phospholipid distribution across bilayer membranes. Med Res Rev 22, 251-281.
Dowler, S., Currie, R. A., Campbell, D. G., Deak, M., Kular, G., Downes, C. P., and Alessi, D. R. (2000). Identification of pleckstrin-homology-domain-containing proteins with novel phosphoinositide-binding specificities. The Biochemical journal 351, 19-31.
Falasca, M., and Maffucci, T. (2006). Emerging roles of phosphatidylinositol 3-monophosphate as a dynamic lipid second messenger. Archives of physiology and biochemistry 112, 274-284
Gonorazky, G., Laxalt, A. M., Testerink, C., Munnik, T., and de la Canal, L. (2008). Phosphatidylinositol 4-phosphate accumulates extracellularly upon xylanase treatment in tomato cell suspensions. Plant, cell & environment 31, 1051-1062.
Hu, Y., Qiao, L., Wang, S., Rong, S. B., Meuillet, E. J., Berggren, M., Gallegos, A., Powis, G., and Kozikowski, A. P. (2000). 3-(Hydroxymethyl)-bearing phosphatidylinositol ether lipid analogues and carbonate surrogates block PI3-K, Akt, and cancer cell growth. J Med Chem 43, 3045-3051.
Lee, S. A., Kovacs, J., Stahelin, R. V., Cheever, M. L., Overduin, M., Setty, T. G., Burd, C. G., Cho, W., And Kutateladze, T. G. (2006). Molecular mechanism of membrane docking by the Vam7p PX domain. J Biol Chem 281, 37091-37101.
Li, W., Schultz, C., Llopis, J., and Tsien, R. Y. (1992). Membrane-permeant esters of inositol polyphosphates, chemical syntheses and biological applications. Tetrahedron 53, 12017-12040.

Martin, F., Aerts, A., Ahren, D., Brun, A., Danchin, E. G., Duchaussoy, F., Gibon, J., Kohler, A., Lindquist, E., Pereda, V., et al. (2008). The genome of Laccaria bicolor provides insights into mycorrhizal symbiosis. Nature 452, 88-92.
Mufti, N. S., Louis, J., Pappan, L. K., Pappan, K., Begum, K., Chen, M. S., Park, Y., Dittmer, N., Marshall, J., Reese, L C., et al. (2008). A protein from the salivary glands of the pea aphid, Acyrthosiphon pisum, is essential in feeding on a host plant. Proceedings of the National Academy of Sciences of the United States of America 105, 9965-9969.
Regente, M. Corti Monzon, G., and de la Canal, L. (2008). Phospholipids are present in extracellular fluids of imbibing sunflower seeds and are modulated by hormonal treatments. Journal of experimental botany 59, 553-562.
Wang, W., Wen, Y., Berkey, R., and Xiao, S. (2009). Specific Targeting of the Arabidopsis Resistance Protein RPW8.2 to the Interfacial Membrane Encasing the Fungal Haustorium Renders Broad-Spectrum Resistance to Powdery Mildew. Plant Cell 21, 2898-2913.

1. A method of inhibiting entry, into a cell, of a pathogenic effector protein, said entry of said pathogenic effector protein into said cell requiring binding of at least one motif of said effector protein to at least one polar lipid of said cell, comprising the step of
i) binding a blocking compound to said at least one motif of said pathogenic effector protein; or
ii) binding a blocking compound to said at least one polar lipid of said cell,
wherein said step of binding prevents entry of said pathogenic effector protein into said cell.

2. The method of claim 1, wherein said at least one motif comprises an amino acid sequence BXZ, where
B is an amino acid selected from arginine, lysine and histidine;
X is any amino acid and may be present or absent; and
Z is an amino acid selected from leucine, methionine, isoleucine, tryptophan, tyrosine and phenylalanine.

3. The method of claim 2, and wherein said at least one motif is selected from the group consisting of RxLR, RSLR, Pexel, RYWT, RIYER, RRLLR, RRFLR, and RFYR.

4. The method of claim 3, wherein said at least one motif is RIYER or RYWT.

5. The method of claim 1, wherein said blocking compound is a polypeptide that binds to said at least one motif.

6. The method of claim 5, wherein said polypeptide is a synthetic peptide.

7. The method of claim 1, wherein said blocking compound is a polar lipid.

8. The method of claim 7 wherein said polar lipid is selected from phosphoinositides, phospholipids, and sphingolipids.

9. The method of claim 8 wherein said phosphoinositide is selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

10. The method of claim 7 wherein said polar lipid is selected from the group consisting of phosphatidyl-serine (PS), phosphatidyl-glycerol (PG), phosphatidyl-ethanolamine (PE), phosphatidyl-choline (PC), lysophosphatidyl-serine (LPS), lysophosphatidyl-glycerol (LPG), lysophosphatidyl-ethanolamine (LPE), lysophosphatidyl-choline (LPC), phosphatidic acid (PA), lysophosphatidic acid (LPA), sphingosine-1-phosphate (S-1-P), ceramide-1-phosphate (C-1-P), a glycosylphosphatidylinositol (GPI)-protein anchor, a glycosylsphingosylinositol (GSI)-protein anchor, a glycosyl phosphoryl inositol ceramide (GPIC) and sphingomyelin (SM).

11. The method of claim 1, wherein said blocking compound is selected from the group consisting of: an inositol phosphate, an inositol sulfate, an inositol carboxylate, an inositol arsenate, an inositol phosphorothioate, a hexose phosphate, a hexose sulfate, a hexose carboxylate, a hexose arsenate, a hexose phosphorothioate, a hexitol phosphate, a hexitol sulfate, a hexitol carboxylate, a hexitol arsenate, a hexitol phosphorothioate, a polyol phosphate, a polyol sulfate, a polyol carboxylate, a polyol arsenate, a polyol phosphorothioate, a phosphorylated glycan, a sulfated glycan, a carboxylated glycan, a glycan arsenate, or a glycan phosphorothioate.

12. The method of claim 11, wherein said blocking compound is a polypeptide that binds to said at least one polar lipid.

13. The method of claim 12, wherein said polypeptide is or comprises a portion of a lipid-binding protein.

14. The method of claim 13, wherein said polypeptide comprises a domain selected from the group consisting of C1, C2, PH, FYVE, PX, ENTH, ANTH, BAR, FERM, PDZ, and tubby domains.

15. The method of claim 12, wherein said polypeptide is a synthetic peptide.

16. The method of claim 1, wherein said host cell is a plant cell.

17. The method of claim 16, wherein said plant cell is of a type selected from the group consisting of wheat, maize, rice, sorghum, barley, oats, millet, soybean, common bean (Phaseolus species), green pea (Pisum species), cowpea, chickpea, alfalfa, clover, tomato, potato, tobacco, pepper, egg plant, grape, strawberry, raspberry, cranberry, blueberry, blackberry, hops, walnut, apple, peach, plum, pistachio, apricot, almond, pear, avocado, cacao, coffee, tea, pineapple, passionfruit, coconut, date and oil palm, citrus, orange, lemon, grapefruit, safflower, carrot, sesame, common bean, banana, citrus, papaya, macadamia, guava, pomegranate, pecan, Brassica species, canola, cabbage, cauliflower, mustard, cucurbits, pumpkin, cantalope, squash, zucchini, melon, cotton, sugar cane, sugar beets, sunflower, lettuce, onion, garlic, ornamental cut flowers; and grass.

18. The method of claim 1 wherein said host cell is an animal cell.

19. The method of claim 18, wherein said animal cell is from or in an animal selected from the group consisting of cattle, sheep, pigs, goats, horses, cats, dogs, chickens, turkeys, bees, salmon, trout, bass, catfish, shellfish, crayfish, lobsters, shrimp, and crabs.

20. The method of claim 19 wherein said animal cell is a human cell.

21. The method of claim 19 wherein said animal cell is a red blood cell, a lymphocyte, a macrophage, a neutrophil, a dendritic cell, a spleen cell, a thymus cell, a liver cell, a nerve cell, a brain cell, a lung cell, a muscle cell, or an epithelial cell.

22. The method of claim 1 wherein said pathogenic effector protein is from an oomycete.

23. The method of claim 22 wherein said oomycete is selected from the group consisting of: a Phytophthora species, Phytophthora infestans, Phytophthora sojae, Phytophthora ramorum, Phytophthora parasitica, Phytophthora capsici, Phytophthora nicotianae, Phytophthora cinnamomi, Phytophthora cryptogea, Phytophthora drechsleri, Phytophthora cactorum, Phytophthora cambivora, Phytophthora citrophthora, Phytophthora citricola, Phytophthora megasperma, Phytophthora palmivora, Phytophthora megakarya, Phytophthora boehmeriae, Phytophthora kernoviae, Phytophthora erythroseptica, Phytophthora fragariae, Phytophthora heveae, Phytophthora lateralis, Phytophthora syringae, a Pythium species, Pythium ultimum, Pythium aphanidermatum, Pythium irregulare, Pythium graminicola, Pythium arrhenomanes, Pythium insidiosum, a downy mildew species, a Peronospora species, Peronospora tabacina, Peronospora destructor, Peronospora sparsa, Peronospora viciae, a Bremia species, Bremia lactucae, a Plasmopora species, Plasmopora viticola, Plasmopara halstedii, a Pseudoperonospora species, Pseudoperonospora cubensis, Pseudoperonospora humuli, a Sclerospora species, Sclerospora graminicola, a Peronosclerospora species, Peronosclerospora philippinesis, Peronosclerospora sorghi, Peronosclerospora sacchari, a Sclerophthora species, Sclerophthora rayssiae, Sclerophthora macrospora, a Albugo species, Albugo candida, a Aphanomyces species, Aphanomyces cochlioides, Aphanomyces euteiches, Aphanomyces invadans, a Saprolegnia species, Saprolegnia parasitica, and a Achlya species.

24. The method of claim 1 wherein said pathogenic effector protein is from a fungus.

25. The method of claim 24 wherein said fungus is selected from the group consisting of: a rust fungus, a smut fungus, a bunt fungus, a powdery mildew fungus, a Puccinia species, Puccinia striiformis, Puccinia graminis, Puccinia triticina (syn. Puccinia recondita), Puccinia sorghi, Puccinia schedonnardii, Puccinia cacabata, a Phakopsora species, Phakopsora pachyrhizi, Phakopsora gossypii, a Phoma species, Phoma glycinicola, a Ascochyta species, Ascochyta gossypii, a Cryphonectria species, Cryphonectria parasitica, a Magnaporthe species, Magnaporthe oryzae, a Gaeumannomyces species, Gaeumannomyces graminis, a Synchytrium species, Synchytrium endobioticum, a Ustilago species, Ustilago maydis, Ustilago tritici, Ustilaginoidea vixens, a Tilletia species, Tilletia indica, Tilletia caries, Tilletia foetida, Tilletia barclayana, a Erysiphe species, Erysiphe necator, a Blumeria species, Blumeria graminis, Podosphaera oxyacanthae, a Alternaria species, Alternaria alternata, a Botrytis species, Botrytis cinerea, a Diaporthe species, Diaporthe phaseolorum, a Fusarium species, Fusarium graminearum, Fusarium oxysporum, Fusarium moniliforme, Fusarium solani, a Leptosphaeria species, Leptosphaeria maculans, Leptosphaeria maydis, a Macrophomina species, Macrophomina phaseolina, a Monilinia species, Monilinia fructicola, a Mycosphaerella species, Mycosphaerella graminicola, Mycosphaerella fijiensis, Mycosphaerella tassiana, Mycosphaerella zeae-maydis, a Phialophora species, Phialophora gregata, a Phymatotrichopsis species, Phymatotrichopsis omnivora, a Taphrina species, Taphrina deformans, a Aspergillus species, Aspergillus flavus, Aspergillus parasiticus, Aspergillus fumigatus, a Verticillium species, Verticillium dahliae, Verticillium albo-atrum, Rhizoctonia solani, Ophiostoma ulmi, Ophiostoma novo-ulmi, a Septoria species, Septoria avenae, a Pyrenophora species, Pyrenophora tritici-repentis, a Colletotrichum species, Colletotrichum graminicola, a Sclerotinia species, Sclerotinia sclerotiorum, a Sclerotium species, Sclerotium rolfsii, a Thielaviopsis species, Thielaviopsis basicola, a Coccidioides species, Coccidioides immitus, a Paracoccidioides species, Paracoccidioides braziliensis, a Pneumocystis species, Pneumocystis carinii, a Histoplasma species, Histoplasma capsulatum, a Cryptococcus species, Cryptococcus neoformans, a Candida species, Candida albicans, a microsporidial species, a Enterocytozoon species, a Encephalitozoon species and Encephalitozoon cuniculi.

26. The method of claim 1, wherein said pathogen effector protein is from a protozoon.

27. The method of claim 26 wherein said protozoon is selected from the group consisting of: an apicomplexan parasite, a Plasmodium species, Plasmodium falciparum, Plasmodium vivax, Plasmodium ovale, Plasmodium malariae, a Babesia species, Babesia bovis, Babesia bigemina, a Cryptosporidium species, Cryptosporidium parvum, a Toxoplasma species, Toxoplasma gondii, a Trypanosomatid species, a Trypanosoma species, Trypanosoma brucei, Trypanosoma cruzi, Trypanosoma congolense, Trypanosoma vivax, a Leishmania species, Leismania donovani, an amebozoan parasite, an Entamoeba species, Entamoeba histolytica, a Mastigamoeba species, a Schistosoma species, a Onchocerca species, and a Giardia species.

28. The method of claim 1 wherein said step of binding includes the step of providing to a plant or animal a sufficient quantity of said blocking compound so that it is present to bind to said target molecule prior to entry of said pathogenic effector protein into cells of said plan or animal.

29. A method for screening compounds to identify whether they are potential blocking compounds for inhibiting entry of pathogenic effector proteins into a cell, comprising the steps of:
providing one or more proteins each of which has one or more motifs which are bound by polar lipids as a prerequisite to translocation;
exposing a candidate compound to said one or more proteins; and
determining whether said candidate compound binds to said one or more motifs of said one or more proteins, and, if binding occurs, determining that said compound is a potential blocking compound for inhibiting entry of pathogenic effector proteins into a cell,
wherein said one or more motifs comprises an amino acid sequence BXZ, where
B is an amino acid selected from arginine, lysine and histidine;
X is any amino acid and may be present or absent; and
Z is an amino acid selected from leucine, methionine, isoleucine, tryptophan, tyrosine and phenylalanine.

30. The method of claim 29 wherein said one or more proteins provided in said providing step are pathogenic effector proteins derived from a bacterial, protozoal, fungal, oomycete or nematode source.

31. A method for screening compounds to identify whether they are potential blocking compounds for inhibiting entry of pathogenic effector proteins into a cell, comprising the steps of:
providing one or more polar lipids, each of which binds to one or more motifs of an effector protein as a prerequisite to translocation of said effector protein into said cell;
exposing a candidate compound to said one or more polar lipids; and
determining whether said candidate compound binds to said one or morepolar lipids, and, if binding occurs, determining that said compound is a potential blocking compound for inhibiting entry of pathogenic effector proteins into said cell,
wherein said one or more motifs comprises an amino acid sequence BXZ, where
B is an amino acid selected from arginine, lysine and histidine;
X is any amino acid and may be present or absent; and
Z is an amino acid selected from leucine, methionine, isoleucine, tryptophan, tyrosine and phenylalanine.

32. The method of claim 31, wherein said effector proteins are pathogenic effector proteins derived from a bacterial, protozoal, fungal, oomycete or nematode source.

33. A method of inhibiting entry, into a cell, of a pathogenic effector protein, said entry of said pathogenic effector protein into said cell requiring binding of at least one motif of said effector protein to at least one polar lipid of said cell, comprising the step of
contacting a substrate which contains or is likely to contain a pathogen comprising said pathogenic effector protein with a blocking compound, said blocking compound being capable of
i) binding to said at least one motif of said pathogenic effector protein; or
ii) binding to said at least one polar lipid of said cell,
wherein binding of said blocking compound prevents entry of said pathogenic effector protein into said cell if said pathogen comes into contact with said substrate.

34. The method of claim 33, wherein said substrate is selected from the group consisting of plants, fabric, water, skin and fur.
